(19) United States
(12) Patent Application Publication
(10) Pub. No.: US 2026/0242398 A1
(43) Pub. Date: Aug. 20, 2026

(54) BENZOPYRIMIDIN-4(3H)-ONES AS PI3K INHIBITORS

(71) Applicant: OnKure, Inc., Boulder, CO (US)

(72) Inventors: James F. Blake, Boulder, CO (US); Mark Laurence Boys, Boulder, CO (US); David A. Mareska, Boulder, CO (US); Joshua Nathaniel Payette, Boulder, CO (US); Christie A. Schulte, Boulder, CO (US); Bryan Yestrepsky, Boulder, CO (US); Qian Zhao, Boulder, CO (US)

(73) Assignee: OnKure, Inc., Boulder, CO (US)

(21) Appl. No.: 18/871,390

(22) PCT Filed: Jun. 6, 2023
(86) PCT No.: PCT/US2023/024567

§ 371 Date: Dec. 3, 2024

Related U.S. Application Data
(60) Provisional application No. 63/350,234, filed on Jun. 8, 2022.

(51) Int. Cl. C07F 7/08; A61K 31/517; A61K 31/695; A61P 35/00; C07D 239/96; C07D 401/04; C07D 403/04

(52) U.S. Cl. CPC C07F 7/0816; A61K 31/517; A61K 31/695; A61P 35/00; C07D 239/96; C07D 401/04; C07D 403/04

(57) ABSTRACT

Novel PI3K inhibitors of the general Formula (1) are described along with methods of their preparation and their use in the treatment of diseases associated with the elevation or activation of the PI3K pathway,

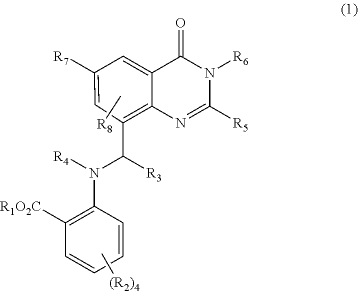

wherein R₁ to R₈ are as defined.

### CROSS-REFERENCE TO RELATED APPLICATIONS

This application claims priority to U.S. Provisional Application No. 63/350,234, filed on Jun. 8, 2022, the contents of which are incorporated herein by reference in its entirety for all purposes.

### BACKGROUND OF THE INVENTION

Phosphatidylinositol lipids (Pls) and their various phosphorylated subspecies are second messengers involved in a wide array of cellular vesicle trafficking and signal transduction processes. Phosphoinositide 3′ kinases (PI3Ks) are a family of enzymes responsible for phosphorylation of the 3′ hydroxyl position of the inositol ring of Pls. PI3Ks are subdivided into 3 classes according to their structure and substrates. Class II PI3Ks (PI3K-C2α, PI3K-C2β, PI3K-C2γ) and Class III PI3Ks (vps34) are monomeric enzymes primarily associated with endocytosis and autophagy (Posor et al., Biochim Biophys Acta 2015, 1851, 794; Backer, Biochem J. 2016, 473, 2251). The Class 1 PI3Ks are heterodimeric, consisting of a catalytic kinase subunit (p110α, β, γ, δ) and one of several regulatory subunits that determine binding partners and subcellular localization. Class 1 PI3Ks are activated upon interaction with receptor tyrosine kinases (RTKs), Ras-related GTPases, G-protein coupled receptors, and/or related adaptor proteins, and in their active form convert phosphatidylinositol 4,5-diphosphate (PIP2) to phosphatidyl 3,4,5-triphosphate (PIP3) (Fruman et al., Cell 2017, 170, 605).

High local concentrations of PIP3 promote the recruitment and activation of downstream signaling partners, including AKT and mTOR. Activation of the AKT/mTOR pathways are implicated in several growth-related roles and pathologies including glucose regulation, cell survival, angiogenesis, and proliferation (Porta et al., Front Oncol. 2014, 4, 1), indicating a role for Class 1 PI3Ks as a critical upstream regulator of these functions.

Class 1 PI3Ks are further subdivided into 4 isoforms (α, β, γ, and δ) based on the identity of their catalytic (p110α, p110β, p110γ, or p110δ) and regulatory (p85α or its various splice variants, p85β, p55γ, or p101) subunits, giving rise to distinct roles in cellular physiology (Vanhaesebroeck et al., J Mol Med (Berl). 2016, 94, 5). PI3Kγ and PI3Kδ are mostly expressed in leukocytes and play an important role in pro-inflammatory pathways (Hawkins et. al., Biochimica et Biophysica Acta 2015, 1851, 882; Okkenhaug et al., Science 2002, 297, 1031; Ali et al., Nature 2004, 431, 1007). PI3Kα and β are more ubiquitously expressed and share similar but not identical roles. For example, PI3Kα has a nonredundant role in angiogenesis (Soler et al., J Exp Med. 2013, 210, 1937), while PI3Kβ is known to serve a specific function in platelet aggregation (Liu et. al., Nat Rev Drug Discov. 2009, 8, 627; Jackson et al., Nat Med. 2005, 11, 507).

Elevation or constitutive activation of the PI3K pathway is one of the most frequent events in human cancers. The PI3K pathway is overactivated through a variety of mechanisms, including activating mutation of PI3K isoforms, up-regulation of PI3K isoforms, loss or inactivation of the tumor suppressor PTEN, or hyperactivation of tyrosine kinase growth factor receptors or other upstream signaling partners (Yang et al., Mol Cancer 2019, 18, 1). Mutations in the gene coding for PI3Kα or mutations which lead to upregulation of PI3Kα have been found to occur in many human cancers such as lung, stomach, endometrial, ovarian, bladder, breast, colon, brain, prostate, and skin cancers (Goncalves et al., N Eng J Med. 2018, 379, 2052). In particular, PIK3CA, the gene encoding the p110α subunit of PI3Kα, is frequently mutated or amplified in a variety of tumor types. Missense mutations occur in all domains of p110α, but cluster in two ‘hot spots’, the most common being E542K and E545K in the helical domain, and H1047R in the kinase domain. Helical domain mutations reduce inhibition of p110α by p85 or facilitate direct interaction of p110α with insulin receptor substrate 1 (IRS1)37, whereas kinase domain mutations increase interaction of p110α with lipid membranes, concomitantly upregulating signaling events. (Thorpe et al., Nat Rev Cancer 2015, 15, 7).

The development of inhibitors for the PI3K pathway has been challenging due to the inability to achieve dosing sufficient for tumor suppression without adverse events. To date PI3K inhibitors in the clinic (alpelisib, buparlisib, copanlisib, duvelisib, idelalisib, pictilisib, taselisib, and others) have caused dose-dependent adverse events such as hyperglycemia, rash, fatigue, diarrhea, etc. (Jiang et al., Mol Biol Rep. 2020, 47, 4587) which are known on-target toxicities. Hyperglycemia is a result of the body not producing enough insulin or aberrant utilization. The pancreas regulates insulin release in response to changes in blood glucose levels, resulting in either glucose uptake by muscle and fat cells when insulin levels are high or gluconeogenesis by the liver when insulin levels are low. Tissue cellular response to insulin requires PI3K signaling through the ubiquitously expressed p110α sub-unit. As a result, pan-PI3K inhibition of the target disrupts glucose metabolism in tissues, leading to insulin resistance (Hopkins et al., Nature 2018, 560, 499). To mitigate adverse events, selective PI3K isoform inhibitors were developed. The severity of the adverse event is dependent on the select isoform, for example PI3Kα inhibitors are associated with hyperglycemia and rash due to the p110α sub-unit role in insulin response (Rugo et al., The Breast 2022, 61, 156). Similarly, use of a selective PI3Kδ inhibitor (idelalisib), where the p110δ sub-unit is highly expressed in immune cells, causes severe diarrhea and colitis. Inhibition with a dual inhibitor (taselisib), a potent PI3Kδ inhibitor possessing modest PI3Kα inhibition led to gastrointestinal (GI) side effects, but a highly selective and potent PI3Kδ inhibitor (umbralisib) reported no GI related adverse events (Gadkar et al., CPT Pharmacometrics Syst Pharmacol. 2021, 11, 616). Such amelioration of adverse events with highly isoform selective and potent inhibitors demonstrates that a strategy to mitigate toxicity by developing mutant selective isoform inhibitors is promising for decreasing the severity of toxicity. Furthermore, selective inhibition of the mutant PI3Kα isoform over wild type may suppress cancer signaling while having minimal effect on PI3K signaling in healthy cells bearing just wild type PI3Kα, leading to a reduction in the toxicities associated with nonselective PI3K inhibition (Castel et al., Nat Cancer 2021 2, 587).

There is currently an interest in developing PI3K inhibitors for cancer therapy (WO 2023/081209, WO 2023/078401, WO 2023/060262, WO 2023/056407, WO 2021/202964). However, there is a continued need for novel potent and selective PI3K inhibitors, either as single agents or as combination therapies, in the treatment of cancer.

### SUMMARY OF THE INVENTION

An aspect of the invention is a compound of Formula (1)

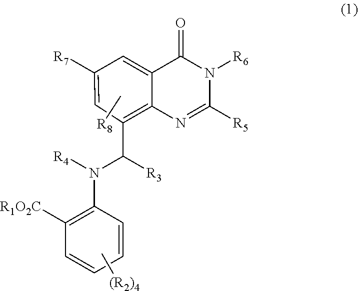

or a solvate, enantiomer, diastereomer, tautomer, polymorph or isotope-labeled compound thereof, or a pharmaceutically acceptable salt thereof,
wherein:
R₁ is H, C₁-C₄ alkyl or C₃-C₇ cycloalkyl;
each R₂ is independently H, C₁-C₄ alkyl, C₃-C₇ cycloalkyl, R₁₄—C≡C—, halogen, CN, CF₃, OCF₃, CFH₂ or CF₂H;
R₃ is H, C₁-C₄ alkyl, C₃-C₇ cycloalkyl, CF₃, CFH₂ or CF₂H, and where R₃ is not H, the carbon atom attached to R₃ is a chiral center and exists as a (R)- and (S)-racemic mixture or as either the (R)- or (S)-enantiomer;
R₄ is H or C₁-C₄ alkyl;
R₆ is H, C₁-C₄ alkyl, C₃-C₇ cycloalkyl, heteroaryl, CF₃, CFH₂ or CF₂H;
R₇ is H, C₁-C₄ alkyl, C₃-C₇ cycloalkyl, halogen, CN, CF₃, OCF₃, OCH₃, CFH₂ or CF₂H;
R₈ is H, C₁-C₄ alkyl, C₃-C₇ cycloalkyl, halogen, CN, CF₃, OCF₃, OCH₃, CFH₂ or CF₂H;
R₅ is

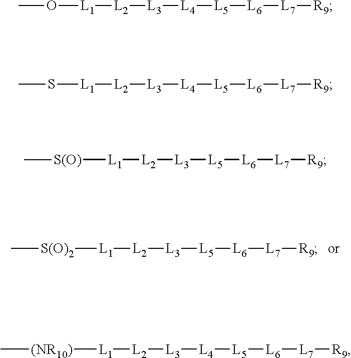

wherein:
each of L₁, L₂, L₃, L₆ and L₇ is independently (CHR₁₁), (CHR₁₁—O), (CHR₁₁—S), (C₃-C₇ cycloalkyl) or a bond;
L₄ is C═O, C═S or a bond;
L₅ is NR₁₀, S, O or a bond;
R₉ is H, C(═O)R₁₂, C(═O)NR₁₂R₁₃, C(═O)OR₂, C₁-C₆ alkyl, C₁-C₆ fluoroalkyl, cycloalkyl, heterocyclyl, aryl or heteroaryl, where each of the C₁-C₆ alkyl, C₁-C₆ fluoroalkyl, cycloalkyl, heterocyclyl, aryl or heteroaryl is unsubstituted or substituted; or alternatively when NR₁₀ is present, R₉ and R₁₀ together with the attached nitrogen atom may form a substituted or unsubstituted ring. In an exemplary embodiment, the ring is 4- to 7-membered substituted or unsubstituted non-aromatic heterocyclic ring containing (in addition to the nitrogen atom) 0, 1 or 2 heteroatoms which may be N, O, S or Si, with the proviso that if the ring size is 4 or 5, the number of additional heteroatoms will be 0 or 1 and if the ring size is from 6 to 7, the number of additional heteroatoms will be 0, 1 or 2, where if the ring is substituted, the substituents include, but are not limited to, one or more of CH₃, F, Cl, CF₃, CF₂H, CH₂F, OCH₃, cyclopropyl, CH₂CF₃, an oxetane ring, or CORₐ where Rₐ is C₁-C₄alkyl, O—C₁-C₄alkyl, or NR_{b}R_{c} where R_{b} and R_{c} are independently H or C₁-C₄alkyl;
each of R₁₀ and R₁₁ is H or C₁-C₄ alkyl, where the C₁-C₄ alkyl is unsubstituted or substituted; and
each of R₁₂ and R₁₃ is independently H or C₁-C₆ alkyl, cycloalkyl, heterocyclyl, aryl or heteroaryl, where each of the C₁-C₆ alkyl, cycloalkyl, heterocyclyl, aryl or heteroaryl is unsubstituted or substituted; or alternatively, R₁₂ and R₁₃ together with the attached nitrogen atom may form a substituted or unsubstituted ring. In an exemplary embodiment, the ring is 4-to 7-membered substituted or unsubstituted non-aromatic heterocyclic ring containing (in addition to the nitrogen atom) 0, 1 or 2 heteroatoms which may be N, O, S or Si, with the proviso that if the ring size is 4 or 5, the number of additional heteroatoms will be 0 or 1 and if the ring size is from 6 to 7, the number of additional heteroatoms will be 0, 1 or 2, where if the ring is substituted, the substituents include, but are not limited to, one or more of CH₃, F, Cl, CF₃, CF₂H, CH₂F, OCH₃, cyclopropyl, CH₂CF₃, an oxetane ring, or CORₐ where Rₐ is C₁-C₄alkyl, O—C₁-C₄alkyl, or NR_{b}R_{c} where R_{b} and R_{c} are independently H or C₁-C₄alkyl; and
each R₁₄ is independently H, C₁-C₃ alkyl or C₃-C₇ cycloalkyl;
or R₅ is
a non-aromatic N-linked heterocyclic ring

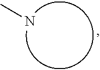

where the heterocyclic ring is substituted or unsubstituted, optionally contains one or more additional atoms selected from N (where the N is substituted or unsubstituted), O, Si (where the Si is substituted or unsubstituted) and S (where the S is oxidized or unoxidized), and is optionally part of a bridged, fused or spiro ring system.

In an exemplary embodiment, R₅ is —NR₁₀-L-L₂-L₃-L₄-L₅-L₆-L₇-R₉, where L₁ to L₇, R₉ and R₁₀ are as defined.

In an exemplary embodiment, R₅ is —O-L-L₂-L₃-L₄-L₅-L₆-L₇-R₉, where L₁ to L₇ and R₉ are as defined.

In an exemplary embodiment, R₅ is —S-L-L₂-L₃-L₄-L₅-L₆-L₇-R₉; —S(O)-L₁-L₂-L₃-L₅-L₆-L₇-R₉; or —S(O)₂-L₁-L₂-L₃-L₅-L₆-L₇-R₉, where L₁ to L₇ and R₉ are as defined.

In an exemplary embodiment, R₉ is a 6-membered aryl ring, or is a 5- to 6-membered heteroaryl ring containing from 1-3 nitrogen atoms; or is a non-aromatic 3- to 7-membered carbocycle; or is a non-aromatic 4- to 7-membered heterocycle containing from 1 to 3 heteroatoms selected from N, O, S and Si with the proviso that if the ring size is 4 or 5 then the number of heteroatoms will be 1 or 2 and if the ring size is 6 or 7 the number heteroatoms will be 1, 2 or 3; or is a C₁-C₆alkyl group, where the aryl ring, the heteroaryl ring, the carbocycle, the heterocycle and the alkyl group are unsubstituted or substituted with one or more of CH₃, F, Cl, CF₃, CF₂H, CH₂F, OCH₃, —CH₂CF₃, cyclopropyl, —CN, N(CH₃)₂, an oxetane ring, a phenyl or phenoxy group optionally substituted with from 1 to 3 halogens (F, Cl or Br) or CH₃ groups, or CORₐ where Rₐ is C₁-C₄alkyl, O—C₁-C₄alkyl or NR_{b}R_{c}, where R_{b} and R_{c} are independently H or C₁-C₄alkyl.

In an exemplary embodiment, R₅ is

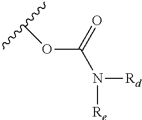

where R_{d} is H or CH₃, and Rₑ is CH₃, C₃-C₆cycloalkyl, a 6-membered aryl ring, or a 5- or 6-membered heteroaryl ring containing from 0, 1 or 2 nitrogen atoms, or R_{d} and Rₑ together with the attached nitrogen atom may form a 4- to 7-membered non-aromatic heterocycle containing (in addition to the nitrogen atom) 0, 1 or 2 heteroatoms which may be N, O, S or Si, with the proviso that if the ring size is 4 or 5, the number of additional heteroatoms will be 0 or 1 and if the ring size is from 6 to 7, the number of additional heteroatoms will be 0, 1 or 2, where the aryl ring, the heteroaryl ring and the heterocycle are unsubstituted or are substituted with one or more that includes, but is not limited to, CH₃, F, Cl, CF₃, CF₂H, CH₂F, OCH₃, cyclopropyl, CH₂CF₃, —CN, N(CH₃)₂, an oxetane ring, a phenyl or phenoxy group optionally substituted with from 1 to 3 halogens (F, Cl or Br) or CH₃ groups, or CORₐ where Rₐ is C₁-C₄alkyl, O—C₁-C₄alkyl or NR_{b}R_{c} where R_{b} and R_{c} are independently H or C₁-C₄alkyl.

In an exemplary embodiment, R₅ is a N-linked non-aromatic heterocyclic ring

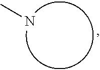

where the heterocyclic ring is substituted or unsubstituted, optionally contains one or more additional atoms selected from N (where the N is substituted or unsubstituted), O, Si (where the Si is substituted or unsubstituted) and S (where the S is oxidized or unoxidized), and is optionally part of a bridged, fused or spiro ring system.

In an exemplary embodiment, the N-linked non-aromatic heterocyclic ring is substituted or unsubstituted, optionally contains one or more additional atoms selected from N, O, Si and S, and is not part of a bridged, fused or spiro ring system.

In an exemplary embodiment, the N-linked non-aromatic heterocyclic ring is substituted or unsubstituted, optionally contains one or more additional atoms selected from N, O, Si and S, and is part of a bridged, fused or spiro ring system.

In an exemplary embodiment, the N-linked non-aromatic heterocyclic ring is substituted or unsubstituted, does not contain additional atoms selected from N, O, Si and S, and is not part of a bridged, fused or spiro ring system.

In an exemplary embodiment, the N-linked non-aromatic heterocyclic ring is substituted or unsubstituted, does not contain additional atoms selected from N, O, Si and S, and is part of a bridged, fused or spiro ring system.

In an exemplary embodiment, the N-linked non-aromatic heterocyclic ring is substituted or unsubstituted, contains at least one sulfur ring atom, and is not part of a bridged, fused or spiro ring system.

In an exemplary embodiment, the N-linked non-aromatic heterocyclic ring is substituted or unsubstituted, contains at least one sulfur ring atom, and is part of a bridged, fused or spiro ring system.

In an exemplary embodiment, the N-linked non-aromatic heterocyclic ring is substituted or unsubstituted, contains at least one oxygen ring atom, and is not part of a bridged, fused or spiro ring system.

In an exemplary embodiment, the N-linked non-aromatic heterocyclic ring is substituted or unsubstituted, contains at least one oxygen ring atom, and is part of a bridged, fused or spiro ring system.

In an exemplary embodiment, the N-linked non-aromatic heterocyclic ring is substituted or unsubstituted, contains at least one additional nitrogen ring atom, and is not part of a bridged, fused or spiro ring system.

In an exemplary embodiment, the N-linked non-aromatic heterocyclic ring is substituted or unsubstituted, contains at least one additional nitrogen ring atom, and is part of a bridged, fused or spiro ring system.

In an exemplary embodiment of the compound of Formula (1), R₁ is H.

In an exemplary embodiment of the compound of Formula (1), each R₂ is H.

In an exemplary embodiment of the compound of Formula (1), R₃ is CH₃.

In an exemplary embodiment of the compound of Formula (1), R₄ is H.

In an exemplary embodiment of the compound of Formula (1), R₆ is CH₃.

In an exemplary embodiment of the compound of Formula (1), R₇ is CH₃ or F.

In an exemplary embodiment of the compound of Formula (1), R₈ is H.

In an exemplary embodiment of the compound of Formula (1), R₁, each R₂ and R₄ are H.

In an exemplary embodiment, the compound of Formula (1) is a compound of Formula (2)

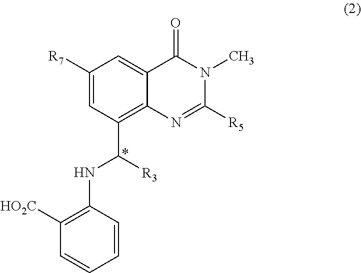

or a solvate, enantiomer, diastereomer, tautomer, polymorph or isotope-labeled compound, or a pharmaceutically acceptable salt thereof, wherein R₃ is CH₃, CF₃, CFH₂ or CF₂H; R₅ is defined as in the compound of Formula (1); R₇ is CH₃ or F; and the carbon atom marked with * is a chiral center and exists as a (R)- and (S)-racemic mixture or as either (R)- or (S)-enantiomer.

In an exemplary embodiment, the compound of Formula (1) is a compound of Formula (3)

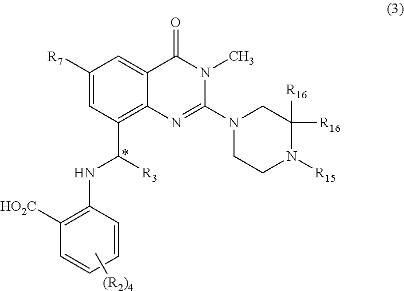

or a solvate, enantiomer, diastereomer, tautomer, polymorph or isotope-labeled compound, or a pharmaceutically acceptable salt thereof, wherein R₂ is defined as in the compound of Formula (1); R₃ is CH₃, CF₃, CFH₂ or CF₂H; R₇ is CH₃ or F; R₁₅ is OCH₃, OCH₂CH₃, OCH₂CF₃, O-cyclopropyl, CH₂CF₃, CH₂CF₂H, aryl (wherein aryl may be, for example, a 6-membered aryl ring), or heteroaryl (wherein heteroaryl may be, for example, a 5- or 6-membered heteroaryl ring or a fused 6,5-heteroaryl ring system such as, for example, benzimidazole, indazole, imidazopyridine or triazolopyridine); each R₁₆ is independently H or C₁-C₃ alkyl; and the carbon atom marked with * is a chiral center and exists as a (R)- and (S)-racemic mixture or as either (R)- or (S)-enantiomer.

In an exemplary embodiment, the compound of Formula (1) is a compound of Formula (4)

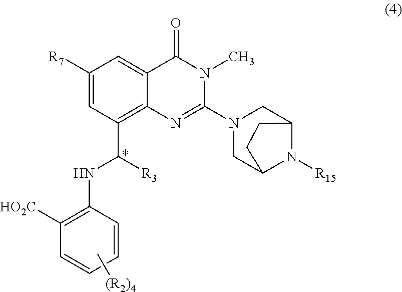

or a solvate, enantiomer, diastereomer, tautomer, polymorph or isotope-labeled compound, or a pharmaceutically acceptable salt thereof, wherein R₂ is defined as in the compound of Formula (1); R₃ is CH₃, CF₃, CFH₂ or CF₂H; R₇ is CH₃ or F; R₁₅ is OCH₃, OCH₂CH₃, OCH₂CF₃, O-cyclopropyl, CH₂CF₃, CH₂CF₂H, aryl (wherein the aryl may be, for example, a 6-membered aryl ring), or heteroaryl (wherein heteroaryl may be, for example, a 5- or 6-membered heteroaryl ring or a fused 6,5-heteroaryl ring system such as, for example, benzimidazole, indazole, imidazopyridine or triazolopyridine); and the carbon atom marked with * is a chiral center and exists as a (R)- and (S)-racemic mixture or as either (R)- or (S)-enantiomer.

In an exemplary embodiment, the compound of Formula (1) is a compound of Formula (5)

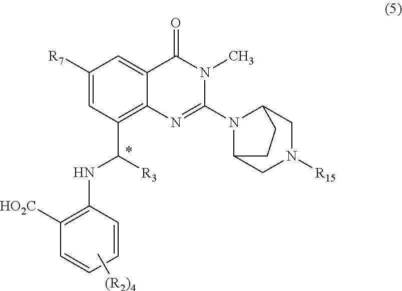

or a solvate, enantiomer, diastereomer, tautomer, polymorph or isotope-labeled compound, or a pharmaceutically acceptable salt thereof, wherein R₂ is defined as in the compound of Formula (1); R₃ is CH₃, CF₃, CFH₂ or CF₂H; R₇ is CH₃ or F; R₁₅ is OCH₃, OCH₂CH₃, OCH₂CF₃, O-cyclopropyl, CH₂CF₃, CH₂CF₂H, aryl (wherein the aryl may be, for example, a 6-membered aryl ring), or heteroaryl (wherein heteroaryl may be, for example, a 5- or 6-membered heteroaryl ring or a fused 6,5-heteroaryl ring system such as, for example, benzimidazole, indazole, imidazopyridine or triazolopyridine); and the carbon atom marked with * is a chiral center and exists as a (R)- and (S)-racemic mixture or as either (R)- or (S)-enantiomer.

An aspect of the invention is a pharmaceutical composition comprising any compound of the invention as described herein (such as any one of Formula (1), (2), (3), (4) or (5)) or a solvate, enantiomer, diastereomer, tautomer, polymorph or isotope-labeled compound thereof, or a pharmaceutically acceptable salt thereof, and a pharmaceutically acceptable carrier.

In an exemplary embodiment, the pharmaceutical composition comprising any compound of the invention as described herein (such as any one of Formula (1), (2), (3), (4) or (5)) or a solvate, enantiomer, diastereomer, tautomer, polymorph or isotope-labeled compound thereof, or a pharmaceutically acceptable salt thereof further comprises one or more anti-cancer agents.

Another aspect of the invention is a method of treating a disease in which PI3K activity is implicated in a subject in need of such treatment, the method comprising administering to the subject a therapeutically effective amount of any compound of the invention as described herein (such as any one of Formula (1), (2), (3), (4) or (5)) or a solvate, enantiomer, diastereomer, tautomer, polymorph or isotope-labeled compound thereof, or a pharmaceutically acceptable salt thereof.

In an exemplary embodiment, the disease to be treated is cancer. In a particular embodiment, the disease is a cancer bearing a PI3Kα H1047 mutation (such as H1047R).

### DETAILED DESCRIPTION OF THE INVENTION

The term “at risk for” as used herein, refers to a medical condition or set of medical conditions exhibited by a patient which may predispose the patient to a particular disease or affliction. For example, these conditions may result from influences that include, but are not limited to, behavioral, emotional, chemical, biochemical, or environmental influences.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, which can be expressed as the ratio LD₅₀/ED₅₀. Compounds that exhibit large therapeutic indices are preferred. The data obtained from these cell culture assays and additional animal studies can be used in formulating a range of dosages for human use. The dosages of such compounds lie preferably within a range of circulating concentrations that include the ED₅₀ with little or no toxicity. The dosage varies within this range depending upon the dosage form employed, the sensitivity of the patient, and the route of administration.

The term “symptom” as used herein, refers to any subjective or objective evidence of disease or physical disturbance observed by the patient. For example, subjective evidence is usually based upon patient self-reporting and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue body imaging scans and other medical testing results.

The term “disease” as used herein, refers to any impairment of the normal state of the living animal or one of its parts that interrupts or modifies the performance of the vital functions. Typically manifested by distinguishing signs and symptoms, a disease is usually a response to i) environmental factors (such as malnutrition, industrial hazards, or climate); ii) specific infective agents (such as worms, bacteria, or viruses); iii) inherent defects of the organism (such as genetic anomalies); and/or iv) combinations of these factors.

The terms “reduce”, “inhibit”, “diminish”, “suppress”, “decrease”, “prevent” and grammatical equivalents thereof (including “lower”, “smaller”, etc.) when used in reference to the expression of any symptom in an untreated subject relative to a treated subject, indicate that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “inhibitory compound” as used herein, refers to any compound capable of interacting with (i.e., for example, attaching, binding, etc.) to a binding partner under conditions such that the binding partner becomes unresponsive to its natural ligands. Inhibitory compounds may include, but are not limited to, small organic molecules, antibodies, and proteins/peptides.

The term “attached” as used herein, refers to any interaction between a medium (or carrier) and a drug. Attachment may be reversible or irreversible. Such attachment includes, but is not limited to, covalent bonding, ionic bonding, Van der Waals forces or friction, and the like. A drug is attached to a medium (or carrier) if it is impregnated, incorporated, coated, in suspension with, in solution with, mixed with, etc.

The term “drug” or “compound” as used herein, refers to any pharmacologically active substance capable of being administered which achieves a desired effect. Drugs or compounds can be synthetic or naturally occurring, non-peptide, proteins or peptides, oligonucleotides or nucleotides, polysaccharides, or sugars.

The term “administered” or “administering” as used herein, refers to any method of providing a composition to a patient such that the composition has its intended effect on the patient. An exemplary method of administering is by a direct mechanism such as, local tissue administration (i.e., for example, extravascular administration, such as subcutaneous, intramuscular, or intraperitoneal), intravenous, oral ingestion, transdermal patch, topical, inhalation, suppository, etc.

The term “patient” as used herein, is a human or animal and needs not be hospitalized. For example, out-patients and persons in nursing homes are “patients.” A patient may be a human or non-human animal of any age and therefore includes both adults and juveniles (i.e., children). It is not intended that the term “patient” connote a need for medical treatment. Therefore, a patient may voluntarily be subject to experimentation, whether clinical or in support of basic science studies.

The term “subject” as used herein, refers to, but is not limited to, humans (e.g., a male or female of any age group, e.g., a pediatric subject (e.g., infant, child, adolescent) or adult subject (e.g., young adult, middle-aged adult or senior adult)) and/or other primates (e.g., monkeys); non-human mammals, such as cows, pigs, horses, sheep, mice, goats, cats, dogs; and/or birds, such as chickens, ducks and/or geese.

The term “affinity” as used herein, refers to any attractive force between substances or particles that causes them to enter into and remain in chemical combination. For example, an inhibitor compound that has a high affinity for a receptor will provide greater efficacy in preventing the receptor from interacting with its natural ligands, than an inhibitor with a low affinity.

The term “derived from” as used herein, refers to the source of a compound or sequence. In one respect, a compound or sequence may be derived from an organism or particular species. In another respect, a compound or sequence may be derived from a larger complex or sequence.

The term “test compound” as used herein, refers to any compound or molecule considered a candidate as an inhibitory compound.

The term “combination therapy” as used herein refers to refers to a dosing regimen of two or more different therapeutically active agents during a period of time, wherein the therapeutically active agents are administered together or separately. In one embodiment the combination therapy is a non-fixed combination.

The term “non-fixed combination” as used herein refers to two or more different therapeutic agents that are formulated as separate compositions or dosages such that they may be administered separately to a subject in need thereof either simultaneously or sequentially with variable intervening time limits.

The term “synergy” or “synergistic” as used herein refers to the phenomenon where the combination of two therapeutic agents of a combination therapy is greater in terms of measured results than the sum of the effect of each agent when administered alone.

The term “in vivo” as used herein refers to an event that takes place in a subject's body.

The term “in vitro” as used herein refers to an event that takes places outside of a subject's body.

The term “protein” as used herein, refers to any of numerous naturally occurring extremely complex substances (such as an enzyme or antibody) that contain amino acid residues joined by peptide bonds, and which include carbon, hydrogen, nitrogen, oxygen, and typically sulfur. In general, a protein comprises amino acids having an order of magnitude within the hundreds.

The term “peptide” as used herein, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens.

The term “pharmaceutically acceptable” or “pharmacologically acceptable” as used herein, refers to molecular entities and compositions that do not produce adverse, allergic, or other untoward reactions when administered to an animal or a human.

The term, “pharmaceutically acceptable carrier” as used herein, includes any and all solvents, or a dispersion medium including, but not limited to, water, ethanol, a polyol (such as, for example, glycerol, propylene glycol, and liquid polyethylene glycol, and the like), suitable mixtures thereof, vegetable oils, coatings, isotonic and absorption delaying agents, liposome, commercially available cleansers, and the like. Supplementary bioactive ingredients also can be incorporated into such carriers.

The term “pharmaceutically acceptable salt” as used herein, refers to a salt that does not adversely impact the biological activity and properties of the compound and is suitable for use in contact with the tissues of subjects without undue toxicity, irritation and/or allergic response and the like. Pharmaceutically acceptable salts include those derived from suitable inorganic acids, organic acids and bases, and include hydrochloric acid, hydrobromic acid, phosphoric acid, sulfuric acid, nitric acid, acetic acid, oxalic acid, maleic acid, tartaric acid, citric acid, succinic acid, malonic acid, ascorbic acid, methanesulfonic acid, ethanesulfonic acid, benzenesulfonic acid, p-toluenesulfonic acid, benzoic acid, naphthalene sulfonic acid, lactic acid, succinic acid, oxalic acid, stearic acid, and the like. In some instances, pharmaceutically acceptable salts are obtained by reacting a compound having acidic group described herein with a base to form a salt such as an ammonium salt, an alkali metal salt (e.g., a sodium or a potassium salt), an alkaline earth metal salt (e.g., a calcium or a magnesium salt), a salt formed from an organic base, and an amino acid salt. Pharmaceutically acceptable salts derived from appropriate bases include alkali metals, alkaline earth metals, and ammonium and quaternary ammonium compounds. Specific metals include, but are not limited to, sodium, lithium, potassium, calcium, magnesium, iron, zinc, copper, manganese, aluminum, and the like. Organic bases from which salts may be prepared include, for example, primary, secondary, and tertiary amines.

The term “prodrug” as used herein, refers to a compound that is transformed in vivo to yield a disclosed compound or a pharmaceutically acceptable form of the compound. A prodrug may be inactive when administered to a subject, but is converted in vivo to an active compound. In various instances, a prodrug has improved physicochemical properties (such as bioavailability) and/or delivery properties over the parent compound. Prodrugs are typically designed to enhance pharmaceutically and/or pharmacokinetically based properties associated with the parent compound. The prodrug compound often offers advantages of solubility, tissue compatibility or delayed release in subject. Prodrugs include compounds wherein a hydroxy, amino, or mercapto group is bonded to any group that, when the prodrug is administered to a subject, cleaves to form a free hydroxy, free amino, or free mercapto group, respectively. Prodrugs are well known to be prepared from carboxylic acids in the form of, for example, carboxylate esters or thioesters.

The term, “purified” or “isolated” as used herein, may refer to a composition (such as, for example, a peptide composition) that has been subjected to treatment (e.g., fractionation) to remove various other components, and which composition substantially retains its expressed biological activity.

The term “sample” as used herein, includes, for example, environmental and biological samples. Environmental samples include material from the environment such as soil and water. Biological samples include animal (e.g., human), fluids (e.g., blood, plasma, and serum), solids (e.g., stool), tissue, liquid foods (e.g., milk), and solid foods (e.g., vegetables). For example, a pulmonary sample may be collected by bronchoalveolar lavage (BAL) which comprises fluid and cells derived from lung tissues. A biological sample may comprise a cell, tissue extract, body fluid, chromosomes or extrachromosomal elements isolated from a cell, genomic DNA (in solution or bound to a solid support such as for Southern blot analysis), RNA (in solution or bound to a solid support such as for Northern blot analysis), cDNA (in solution or bound to a solid support) and the like.

The term “biologically active” as used herein, refers to any molecule having structural, regulatory or biochemical functions. For example, biological activity may be determined, for example, by restoration of wild-type growth in cells lacking protein activity. Cells lacking protein activity may be produced by many methods (i.e., for example, point mutation and frame-shift mutation). Complementation is achieved by transfecting cells which lack protein activity with an expression vector which expresses the protein, a derivative thereof, or a portion thereof.

The term “label” or “detectable label” as used herein, refers to any composition detectable by spectroscopic, photochemical, biochemical, immunochemical, electrical, optical or chemical means. Such labels include biotin for staining with labeled streptavidin conjugate, magnetic beads (e.g., Dynabeads®), fluorescent dyes (e.g., fluorescein, Texas Red®, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include, but are not limited to, U.S. Pat. Nos. 3,817,837; 3,850,752; 3,939,350; 3,996,345; 4,277,437; 4,275,149; and 4,366,241 (all herein incorporated by reference in their entireties). The labels contemplated in the present invention may be detected by conventional methods. For example, radiolabels may be detected using photographic film or scintillation counters, fluorescent markers may be detected using a photodetector to detect emitted light. Enzymatic labels are typically detected by providing the enzyme with a substrate and detecting, the reaction product produced by the action of the enzyme on the substrate, and calorimetric labels are detected by simply visualizing the colored label.

The term “conjugate” as used herein, refers to any compound that has been formed by the joining of two or more moieties.

A “moiety” or “group” as used herein, is any type of molecular arrangement designated by formula, chemical name, or structure. Within the context of certain embodiments, a conjugate comprises one or more moieties or chemical groups. This means that the formula of the moiety is substituted at some position in order to be joined and be a part of the molecular arrangement of the conjugate. Although moieties may be directly covalently joined, it is not intended that the joining of two or more moieties must be directly to each other. A linking group, a crosslinking group, or a joining group refers to any molecular arrangement that will connect moieties by covalent bonds such as, but not limited to, one or more amide group(s). Additionally, although the conjugate may be unsubstituted, the conjugate may have a variety of additional substituents connected to the linking groups and/or connected to the moieties.

A “polymer” or “polymer group” as used herein, refers to a chemical species or group composed of repeatedly linked moieties. Within certain embodiments, it is preferred that the number of repeating moieties is 3 or more or greater than 10. The linked moieties may be identical in structure or may vary in their moiety structures. A “monomeric polymer” or “homopolymer” is a polymer that contains the same repeating, asymmetric subunit. A “copolymer” is a polymer derived from two or more types of monomeric species (i.e., two or more different chemical asymmetric subunits). “Block copolymers” are polymers comprised of two or more species of polymer subunits linked by covalent bonds.

The term “substituted” as used herein, refers to at least one hydrogen atom of a molecular arrangement that is replaced with a substituent. The number of substituents present depends on the number of hydrogen atoms available for replacement and includes replacement of more than one hydrogen atom bound to a single atom (such as in the case of a carbon atom or a silicon atom which may be available for mono-, di- or tri-substitution or in the case of a nitrogen atom which may be available for mono-, di- or tri-substitution or in the case of an oxygen atom or a sulfur atom which may be available for mono-substitution). In the case of an oxo substituent (“═O”), two hydrogen atoms are replaced (which provides, for example, —(CH₂)—C(═O)—CH₃ as a substituent when the two hydrogen atoms of the middle carbon atom of —CH₂—CH₂—CH₃ are replaced). When substituted, one or more of the groups below are “substituents.” Substituents include, but are not limited to, halogen (e.g., F, Cl, Br, I), hydroxy (OH), oxo, cyano (CN), nitro (NO₂), amino, alkylamino, dialkylamino, branched or unbranched alkyl (e.g., methyl, ethyl, propyl, isopropyl, sec-butyl, etc.), cycloalkyl (e.g., cyclopropyl), fluoroalkyl (e.g., CF₃, CF₂H, CH₂F, CH₂CF₃, CH₂CF₂H, CHFCHF₂, CF₂CH₂F, CF₂CF₃, CF₂CH₃, CF(CH₃)₂, CH₂CH₂CF₃, CF₂CH₂CF₃, CF₂CF₂CF₃, etc.) or more generally, haloalkyl (e.g., CH₂Cl, CH(CH₃)Br, etc.), O-alkyl (alkoxy) (e.g., OCH₃, OCH₂CH₃, OCH(CH₃)₂, etc.), O-cycloalkyl (e.g., O-cyclopropyl), O-haloalkyl (e.g., OCF₂H, OCFH₂, OCF₃, OCH₂CF₃, OCH₂CF₂H, OCHFCHF₂, OCF₂CH₂F, OCF₂CF₃, OCF₂CH₃, OCF(CH₃)₂, OCH₂CH₂CF₃, OCF₂CH₂CF₃, OCF₂CF₂CF₃ or OCH₂Cl), O-aryl (e.g., O-phenyl), O-heteroaryl, O-heterocyclyl, thioalkyl (e.g., S—CH₃), hydroxyalkyl (e.g., CH₂OH), alkyl ether (e.g., CH₂OCH₃), alkynyl (e.g., —C≡CR_{f}), alkenyl (e.g., —CR_{f}═CR_{f}R_{g}), aryl (e.g., phenyl), arylalkyl (e.g., CH₂Ph), heteroaryl (e.g., pyridyl or any 5- or 6-membered heteroaryl ring), heteroarylalkyl (e.g., CH₂-pyridine), heterocyclyl, heterocycloalkyl and as well as, —NR_{f}R_{g}, —NR_{f}C(═O)R_{g}, .NR_{f}C(═O)NR_{f}NR_{g}, —NR_{f}—C(═O)OR_{f}SO₂R_{g}, —C(═O)R_{f}, —C(═O)OR_{f}, —OR_{f}, —C(═O)NR_{f}R_{g}, —OC(═O)NR_{f}R_{g}, —SR_{f}, —SOR_{f}, —S(═O)₂R_{f}, —OS(═O)₂R_{f} and —S(═O)OR_{f}, where each R_{f} and R_{g} may be the same or different and are independently, hydrogen, alkyl (e.g., CH₃), substituted alkyl, haloalkyl, aryl, substituted aryl, arylalkyl, substituted arylalkyl, heterocyclyl, substituted heterocyclyl, heterocycloalkyl, substituted heterocycloalkyl, heteroaryl or substituted heteroaryl. In addition, the above substituents may be further substituted with one or more of the above substituents, such that the substituent may constitute, for example, a substituted alkyl, a substituted aryl, a substituted arylalkyl, a substituted heterocyclyl, or a substituted heterocycloalkyl.

The term “unsubstituted” as used herein, refers to any compound that does not contain extra substituents attached to the compound. An unsubstituted compound refers to the chemical makeup of the compound without extra substituents (e.g., no non-hydrogen substituents). For example, unsubstituted proline is a proline amino acid even though the amino group of proline may be considered as disubstituted with alkyl groups.

The term “bond” as used herein in describing a substituent with atoms on both sides, refers to the absence of that substituent. For example, in the 4-atom sequence A-B-C-D, when B and C are both listed as being bonds, the result is the 2-atom sequence A-D. If only B is listed as being a bond, the result is the 3-atom sequence A-C-D.

The term “alkyl” as used herein, refers to any straight chain or branched, non-cyclic or cyclic, unsaturated or saturated aliphatic hydrocarbon containing from 1 to 10 carbon atoms, while the term “lower alkyl” has the same meaning as alkyl but contains from 1 to 3 carbon atoms. The term “higher alkyl” has the same meaning as alkyl but contains from 4 to 10 carbon atoms. Representative saturated straight chain alkyls include, but are not limited to, methyl, ethyl, n-propyl, n-butyl, n-pentyl, n-hexyl, n-heptyl, n-octyl, n-nonyl, and the like, while saturated branched alkyls include, but are not limited to, isopropyl, sec-butyl, isobutyl, tert-butyl, isopentyl, and the like. As used herein, a methyl substituent may be depicted as “CH₃” or “Me” or as a terminal bond with no indication of specific atoms.

The term “cycloalkyl” as used herein, refers to saturated and unsaturated cyclic alkyls. Representative saturated cyclic alkyls include, but are not limited to, C₃-C₁₄ (such as C₃-C₇) cycloalkyls, such as cyclopropyl, cyclobutyl, cyclopentyl, cyclohexyl, cycloheptyl, cyclooctyl, cyclododecyl, and the like; while unsaturated cyclic alkyls include, but are not limited to, cyclobutenyl, cyclopentenyl and cyclohexenyl, cyclohexadiene, and the like. Cyclic alkyls are also referred to herein as “homocycles” or “homocyclic rings”.

The term “spiro” or “spirocyclic” as used herein, refers to chemical structures having at least two rings sharing one common atom. The rings may be cycloalkyl, heterocyclyl or a combination thereof, and may include one or more aryl or heteroaryl rings. Exemplary embodiments include 1,4-dioxaspiro[4.5]decane, spirocyclic azetidines and spirocyclic pyrrolidines and spirocyclic piperidines, where the other ring is cycloalkyl (e.g., cyclobutane, cyclopentane or cyclohexane) or heterocyclyl (e.g., piperidine, tetrahydropyran, tetrahydrofuran, azetidine or pyrrolidine).

The term “bicyclic compounds” as used herein, encompasses “bridged” compounds, “fused” compounds and “spiro” compounds as described.

The term “bridged” as used herein, refers to a compound containing two nonadjacent atoms common to two rings. Exemplary embodiments include, but are not limited to, norbornane, bicyclo[1.1.1]pentane, bicyclo[2.2.1]heptane, 1,4-diazabicyclo[2.2.2]octane, other bridged piperazines and bridged piperidines.

The term “fused” as used herein, refers to polycyclic ring systems in which any two adjacent rings have two, and only two, adjacent atoms in common (ortho-fused) and polycyclic ring systems in which a ring contains two, and only two, adjacent atoms in common with each of two or more rings of a contiguous series of ortho-fused rings (ortho- and peri-fused). An exemplary embodiment is pentalene and dibenzoxepine (ortho-fused) and pyrene (ortho- and peri-fused). Ortho-fused systems have “n” common sides and “2n” common atoms while peri-fused systems have “n” common sides and less than “2n” atoms in common. Other exemplary fused systems include fused cyclopropyl rings, fused aziridines and fused azetidines, such as when these rings are fused to a pyrrolidine ring. Other examples include two fused pyrrolidine rings (octahydropyrrolo[3,4-c]pyrrole), fused pyridine rings, such as a pyridine ring fused with a cycloalkyl (e.g., cyclopentane) or with a heterocyclyl (e.g., tetrahydrofuran or tetrahydropyran).

The term “aromatic” or “aryl” as used herein, refers to any aromatic carbocyclic (i.e., all of the ring atoms are carbon) substituent such as, but not limited to, phenyl (from benzene), tolyl (from toluene), xylyl (from xylene) or multi-ring systems (e.g., naphthyl (from naphthalene) and anthracenyl (from anthracene).

The term “arylalkyl” or “aralkyl” as used herein, refers to any alkyl having at least one alkyl hydrogen atom replaced with an aryl moiety such as, but not limited to, benzyl, —(CH₂)₂phenyl, —(CH₂)₃phenyl, —CH(phenyl)₂, and the like.

The term “halogen” as used herein, refers to any fluoro, chloro, bromo, or iodo moiety.

The term “haloalkyl” as used herein, refers to any alkyl where at least one hydrogen atom (and including all hydrogen atoms) has been replaced with a halogen atom, such as, for example, trifluoromethyl, dichloromethyl, difluoromethyl, monofluoromethyl, monobromomethyl, 1,1,1-trifluoroethyl and the like.

The term “heteroaromatic” or “heteroaryl” as used herein, refers to any aromatic heterocyclic ring of 5 to 10 or more members and having at least one heteroatom selected from nitrogen, oxygen or sulfur, and containing at least 1 carbon atom, including, but not limited to, both mono- and bicyclic-ring systems. The heteroaryl ring may be attached as a substituent via a ring heteroatom or a carbon atom. Representative heteroaromatics include, but are not limited to, furan, benzofuran, thiophene, benzothiophene, pyrrole, indole, isoindole, 7-azaindole, 4-azaindole, 5-azaindole, 6-azaindole, 7-azaindazole, pyridine, quinoline, isoquinoline, oxazole, isoxazole, benzoxazole, pyrazole, imidazole, benzimidazole, thiazole, benzothiazole, isothiazole, 1,2,4-triazole, 1,2,3-triazole, tetrazole, 1,2,5-oxadiazole, 1,2,3-oxadiazole, 1,3,4-thiadiazole, pyridazine, pyrimidine, pyrazine, 1,2,4-triazine, 1,3,5-triazine, cinnoline, phthalazine, quinazoline, 1,8-naphthylpyridine, pyrido[3,2-d]pyrimidine, pyrido[4,3-d]pyrimidine, pyrido[3,4-b]pyrazine, pyrido[2,3-b]pyrazine, pteridine, triazolo-pyridines and the like.

The term “heteroarylalkyl” as used herein, means any alkyl having at least one alkyl hydrogen atom replaced with a heteroaryl moiety, such as —CH₂pyridinyl, —CH₂pyrimidinyl, and the like.

The term “heterocycle” or “heterocyclyl” or “heterocyclic ring” as used herein, refers to a nonaromatic ring which is either saturated or unsaturated and which contains 1 or more heteroatoms independently selected from nitrogen, oxygen, sulfur and silicon, wherein each of the nitrogen and sulfur heteroatoms may be in an oxidized state, and each of the nitrogen and silicon heteroatoms is substituted or unsubstituted and the nitrogen heteroatoms may be optionally quaternized, and includes bicyclic rings in which any of the above heterocycles are fused to an aryl or heteroaryl ring. The heterocyclic ring may be attached as a substituent via a ring heteroatom or a carbon atom. In various embodiments, heterocycles may contain 3 to 14 or more ring atoms (such as 3- to 7-membered monocyclic rings or 7- to 10-membered bicyclic rings) and include, but are not limited to, 2H-azirine, azetidine, 2,3-dihydroazete, 1,3-diazetidine, 2H-oxete, thietane, 2H-thiete, azetidin-2-one, morpholine, thiomorpholine, pyrrolidinone, pyrrolidinine, 2-pyrroline, 3-pyrroline, pyrazolidine, 2-pyrazoline, 2-imidazoline, imidazolidine, piperidine, piperazine, ethylene oxide (oxirane), ethylene imine (aziridine), ethylene sulfide (thiirane), oxetane, propylene oxide, 1,3-dioxolane, 1,2-oxathiolane, 1,3-oxathiolane, sulfolane, 2,4-thiazolidinedione, succinimide, 2-oxazolidone, dioxane, hydantoin, valerolactam, tetrahydrofuran, tetrahydropyran, 2H-pyran, 4H-pyran, thiane, 2H-thiopyran, 1,3-dithiane, 1,4-dithiane, 1,3,5-trithiane, pyrrolizidine, 1,4,5,6-tetrahydrocyclopenta[b]pyrrole, tetrahydropyridine, tetrahydropyrimidine, tetrahydrothiophene, tetrahydrothiopyran, indoline, isoindoline, decahydroisoquinoline, decahydroquinoline, 1,2,3,4-tetrahydroquinoline, 1,2-dihydroquinoline, 2H-benzo[e][1,3]oxazine, 2H-benzo[b][1,4]oxazine, quinolin-2(1H)-one, isoquinolin-1(2H)-one, quinuclidine, 1-azaadamantane, 2-azaadamantane, 2,3-dihydroazepine, 2,5-dihydroazepine, oxepane, azonane, spiro[cyclobutane-1,3′-indole], 1-oxaspiro[4,5]decane, 1,6-dioxaspiro[3,4]octane, 2-oxa-7-azaspiro[3,5]nonane, 1,4-dioxa-7-azaspiro[4,4]nonane, 1,3-diazaspiro[4,4]non-2-en-4-one, 2,9-diazaspiro[5,5]undecan-1-one, 8-azaspiro[4,5]decane-7,9-dione, 1,4-dithia-7-azaspiro[4,4]nonane, and the like.

The term “heterocycloalkyl” as used herein, refers to any alkyl having at least one alkyl hydrogen atom replaced with a heterocycle, such as —CH₂morpholinyl, and the like.

The term “alkylamino” as used herein, means at least one alkyl moiety attached through a nitrogen bridge (i.e., —N-(alkyl)ₙ, where n=1 or 2, such as alkylamino or dialkylamino) including, but not limited to, methylamino, ethylamino, dimethylamino, diethylamino, and the like.

The term “alkyloxy” or “alkoxy”, as used herein, means any alkyl moiety attached through an oxygen bridge (i.e., —O-alkyl) such as, but not limited to, methoxy, ethoxy, and the like.

The term “thioalkyl” as used herein, means any alkyl moiety attached through a sulfur bridge (i.e., —S-alkyl) such as, but not limited to, methylthio, ethylthio, and the like.

The term “alkenyl” as used herein, refers to an unbranched or branched hydrocarbon chain having one or more carbon-carbon double bonds therein and may also be referred to as an “unsaturated alkyl”. The double bond of an alkenyl group can be unconjugated or conjugated to another unsaturated group. Suitable alkenyl groups include, but are not limited to vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. An alkenyl group can be unsubstituted or substituted with one or two suitable substituents.

The term “alkynyl” as used herein, refers to unbranched or branched hydrocarbon chain having one or more carbon-carbon triple bonds therein and may also be referred to as an “unsaturated alkyl”. The triple bond of an alkynyl group can be unconjugated or conjugated to another unsaturated group. Suitable alkynyl groups include, but are not limited to ethynyl, propynyl, butynyl, pentynyl, hexynyl, methylpropynyl, 4-methyl-1-butynyl, 4-propyl-2-pentynyl-, and 4-butyl-2-hexynyl. An alkynyl group can be unsubstituted or substituted with one or two suitable substituents.

As used herein, “reactive groups” refer to nucleophiles, electrophiles, or radically active groups, i.e., groups that react in the presence of radicals. A nucleophile is a moiety that forms a chemical bond to its reaction partner (the electrophile) by donating both bonding electrons. Electrophiles accept these electrons. Nucleophiles may take part in nucleophilic substitution, whereby a nucleophile becomes attracted to a full or partial positive charge on an element and displaces the group it is bonded to. Alternatively, nucleophiles may take part in substitution of carbonyl group. Carboxylic acids are often made electrophilic by creating succinyl esters and reacting these esters with aminoalkyls to form amides. Other common nucleophilic groups are thiolalkyls, hydroxylalkyls, primary and secondary amines, and carbon nucleophiles such as enols and alkyl metal complexes. Other preferred methods of ligating proteins, oligosaccharides and cells using reactive groups are disclosed (Lemieux et al., Trends in Biotechnology 1998, 16, 506, incorporated herein by reference in its entirety). In yet another preferred method, one provides reactive groups for the Staudinger ligation, i.e., “click chemistry” with an azide comprising moiety and alkynyl reactive groups to form triazoles. Michael additions of a carbon nucleophile enolate with an electrophilic carbonyl, or the Schiff base formation of a nucleophilic primary or secondary amine with an aldehyde or ketone may also be utilized. Other methods of bioconjugation are provided (Hang et al. Accounts of Chemical Research 2001, 34, 727, and Kiick et al. Proc Natl Acad Sci U.S.A. 2002, 99, 19, both of which are incorporated by reference in its entirety).

The term “biocompatible” as used herein, refers to any material that does not illicit a substantial detrimental response in the host. There is always concern when a foreign object is introduced into a living body that the object will induce an immune reaction, such as an inflammatory response that will have negative effects on the host. In the context of this invention, biocompatibility is evaluated according to the application for which it was designed: for example, a bandage is regarded as biocompatible with the skin, whereas an implanted medical device is regarded as biocompatible with the internal tissues of the body. Preferably, biocompatible materials include, but are not limited to, biodegradable and biostable materials. A substantial detrimental response has not occurred if an implant comprising the material is in close association to its implant site within the host animal and the response is better than a tissue response recognized and established as suitable from materials provided in an ASTM. ASTM subcommittee F04.16 on Biocompatibility Test Methods has developed biocompatibility standards for medical and surgical materials and devices which includes E1262-88, F612-20, F719-20e1, F720-17, F748-16, F749-20, F750-20, F756-17; F763-04, F813-20, F895-11, F981-04, F1027-86, F1408-20a, F1439-03, F1877-16, F1903-18, F1904-14, F1983-14, F1984-99, F2147-01, F2148-18, F2382-18, F2808-17, F1288-19 and F2909-19, each of which is incorporated herein by reference. For example, materials that are to be used in contact with the blood stream must be composed of materials that meet hemocompatibility standards. One of these tests is for damage to red blood cells, which can result in hemolysis that is, rupturing of the cells, as described in F756-17 Standard Practice for Assessment of Hemolytic Properties of Materials.

As used herein, a “bioactive substance” refers to any of a variety of chemical moieties and that binds with a biomolecule such as, but not limited to, peptides, proteins, enzymes, receptors, substrates, lipids, antibodies, antigens, and nucleic acids. In certain preferred embodiments, the bioactive substance is a biomolecule but it is not intended that the bioactive substance be limited to biomolecules. In other preferred embodiments, the bioactive substances provide hydrophobic, hydrophilic, or electrostatic interactions, such as polycarboxylic acids that are anionic at physiological pH. In other preferred embodiment, the alkaline growth factors (with isoelectric point above 7) are retained via favorable electrostatic interactions by the polycarboxylates, and subsequently released in a controlled and sustained manner.

“Cancer” is a term used for a physiological condition in mammals that is typically characterized by unregulated cell growth. Examples of cancer include, but are not limited to, carcinoma, lymphoma, leukemia, blastoma, and sarcoma. More particular examples of such cancers include squamous cell carcinoma, small cell lung cancer, non-small cell lung cancer (NSCLC), glioma, Hodgkin's lymphoma, non-Hodgkin's lymphoma, acute myeloid leukemia (AML), multiple myeloma, gastrointestinal cancer, renal cell carcinoma, renal cancer (e.g., advanced renal cell carcinoma), ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, urothelial carcinoma (including local advanced or metastatic urothelial carcinoma), bladder cancer, hepatoma, breast cancer and head and neck cancer.

The term “stereoisomer” refers to compounds that have the same atomic connectivity but different atomic arrangement in space. Stereoisomers include cis-trans isomers, E and Z isomers, enantiomers, diastereomers and atropisomers. In the context of the present invention, the term “enantiomerically pure” is understood to mean that the compound in question with respect to the absolute configuration of the chiral center is present in an enantiomeric excess of more than 95%, preferably more than 97%.

The present disclosure contemplates all such compounds, including cis and trans isomers, (−)- and (+)-enantiomers, (R)- and (S)-enantiomers, diastereomers isomers, (D)-isomers, (L)-isomers, atropisomers, tautomers and racemic and other mixtures thereof, such as enantiomers or diastereomeric enriched mixtures, all of which are within the scope of the present disclosure. Insofar as compounds of the invention as defined herein may exist in optically active or racemic forms by virtue of one or more asymmetric carbon atoms, the invention includes in its definition any such optically active or racemic form. The synthesis of optically active compounds may be carried out by standard techniques of organic chemistry well known in the art such as, for example, by synthesis from optically active starting materials or by resolution of a racemic compound. Similarly, the enantiomeric or diastereomeric purity of a compound may be evaluated using standard laboratory techniques.

The pharmaceutical compositions of the invention can take any suitable form for the desired route of administration. Where the composition is to be administered orally, any suitable orally deliverable dosage form can be used, including without limitation water, glycols, oils, alcohols, and the like in the case of oral liquid preparations such as suspensions, syrups, elixirs, emulsions, and solutions; or solid carriers such as starches, sugars, kaolin, diluents, lubricants, binders, disintegrating agents, and the like in the case of powders, pills, capsules, and tablets. Because of their ease in administration, tablets and capsules represent the most advantageous oral dosage unit forms. Injectable compositions or intravenous infusions are also provided in the form of solutions, suspensions, and emulsions. For parenteral compositions, the carrier usually comprises sterile water and possibly other ingredients to aid solubility. Injectable solutions may be prepared in which the carrier comprises a saline solution, a glucose solution, or a mixture of a saline and a glucose solution. Suitable oils include, for example, peanut oil, sesame oil, cottonseed oil, corn oil, soybean oil, synthetic glycerol esters of long chain fatty acids, and mixtures of these and other oils. In compositions suitable for percutaneous administration, the carrier optionally comprises a penetration enhancing agent and/or a suitable wetting agent, optionally combined with suitable additives as needed, where the additives may facilitate administration of the composition to the skin and/or may facilitate preparation of the compositions to be delivered. These compositions may be administered in various ways, e.g., as a transdermal patch or as an ointment. Acid or base addition salts of the compounds of the invention are typically more suitable in the preparation of aqueous compositions due to their increased water solubility over the corresponding neutral form of the compounds.

The pharmaceutical compositions of the invention may comprise one or more of a filler, diluent, adjuvant, vehicle, or other excipient to facilitate storage and/or administration of the active ingredients contained therein.

In an exemplary embodiment, a pharmaceutical composition according to the present invention may contain one or more additional therapeutic agents, for example, to increase efficacy or to decrease undesired side effects. In a particular embodiment, the pharmaceutical composition further contains one or more additional therapeutic agents useful to treat or inhibit a disease mediated directly or indirectly by PI3K. Examples of such agents include, without limitation, agents to treat or inhibit cancer, Huntington's disease, cystic fibrosis, liver fibrosis, renal fibrosis, pulmonary fibrosis, skin fibrosis, rheumatoid arthritis, diabetes, or heart failure.

In a specific embodiment, the additional therapeutic agent to be included is an anti-cancer agent. Examples of an anti-cancer agent include, but are not limited to, DNA-damaging cytotoxic drugs, alkylating agents such as cyclophosphamide, dacarbazine, and cisplatin; anti-metabolites such as methotrexate, mercaptopurine, thioguanine, fluorouracil, and cytarabine; plant alkaloids such as vinblastine and paclitaxel; antitumor antibiotics such as doxorubicin, bleomycin and mitomycin; hormones/antihormones such as prednisone, tamoxifen, and flutamide; other types of anticancer agents such as asparaginase, rituximab, trastuzumab, imatinib, retinoic acid, and derivatives, colony stimulating factors, amifostine, camptothecin, topotecan, thalidomide analogs such as lenalidomide, and proteasome inhibitors such as Velcade.

In another embodiment, the present invention provides a method of inhibiting or treating diseases arising from abnormal cell proliferation and/or differentiation in a subject in need thereof, comprising administering to the subject a therapeutically effective amount of one or more compounds according to the present invention. In one embodiment, the method of inhibiting or treating disease comprises administering to a subject in need thereof, a composition comprising an effective amount of one or more compounds of the invention and a pharmaceutically acceptable carrier. The composition to be administered may further contain a therapeutic agent such as an anti-cancer agent.

The compounds of the invention are defined herein by their chemical structures and/or chemical names and are generally listed according to the IUPAC or CAS nomenclature system. Abbreviations that are well known to one of ordinary skill in the art may be used. When a compound is referred to by both a chemical structure and a chemical name, and the chemical structure and chemical name conflict, the chemical structure is intended to be determinative of the compound's identity.

The present invention includes compounds labeled with various radioactive or nonradioactive isotopes. Examples of atomic isotopes may include, but are not limited to, deuterium (²H), tritium (³H), iodine-125 (¹²⁵I), carbon-14 (¹⁴C), nitrogen-15 (¹⁵N), sulfur-35 (³⁵S) and chlorine-36 (³⁶Cl). In an exemplary embodiment, one or more hydrogen atoms in a compound of the invention can be replaced by deuterium. In various embodiments, a compound of the invention includes at least one deuterium atom, or two or more deuterium atoms, or three or more deuterium atoms, etc. As described herein, compounds of the invention may also be radiolabeled with a radioactive isotope such as tritium (³H), iodine-125 (¹²⁵I), and carbon-14 (¹⁴C). A radiolabeled compound is useful as a therapeutic or prophylactic agent, provides a reagent for research such as for an assay, and/or provides a diagnostic agent for techniques such as in vivo imaging. Synthetic methods for incorporating isotopes into organic compounds are well known in the art.

In an embodiment of the invention, a compound of the invention as defined herein (such as a compound of any one of Formula (1), (2), (3), (4) or (5)) or a pharmaceutically-acceptable salt thereof, exists as a single enantiomer being in an enantiomeric excess (% ee) of ≥95%, such as ≥98%, such as ≥99%.

In an embodiment of the invention, a pharmaceutical composition comprises a compound of the invention as defined herein (such as a compound of Formula I) or a pharmaceutically-acceptable salt thereof, where the compound exists as a single enantiomer being in an enantiomeric excess (% ee) of ≥95%, such as ≥98%, such as ≥99%.

In an exemplary embodiment of the invention, the disease or disorder to be treated by the compounds of the invention is selected from congenital lipomatous overgrowth, vascular malformations, epidermal naevi, scoliosis/skeletal and spinal syndrome (CLOVES), mosaic tissue overgrowth syndromes, venous malformations and brain malformations associated with severe epilepsy or PIK3CA-related overgrowth syndrome (PROS) (Keppler-Noreuil et al., Am J Med Genet A. 2015, 167A, 287; Kurek et al Am. J. Hum. Genet. 2012, 90, 1108).

In an exemplary embodiment of the invention, the cancer to be treated is a cancer bearing a PI3K H1047 mutation (such as H1047R) (Thorpe et al., Nat Rev Cancer 2015, 15, 7).

Compounds of Formula I of the present invention were generally prepared according to the synthetic route identified in the below Scheme:

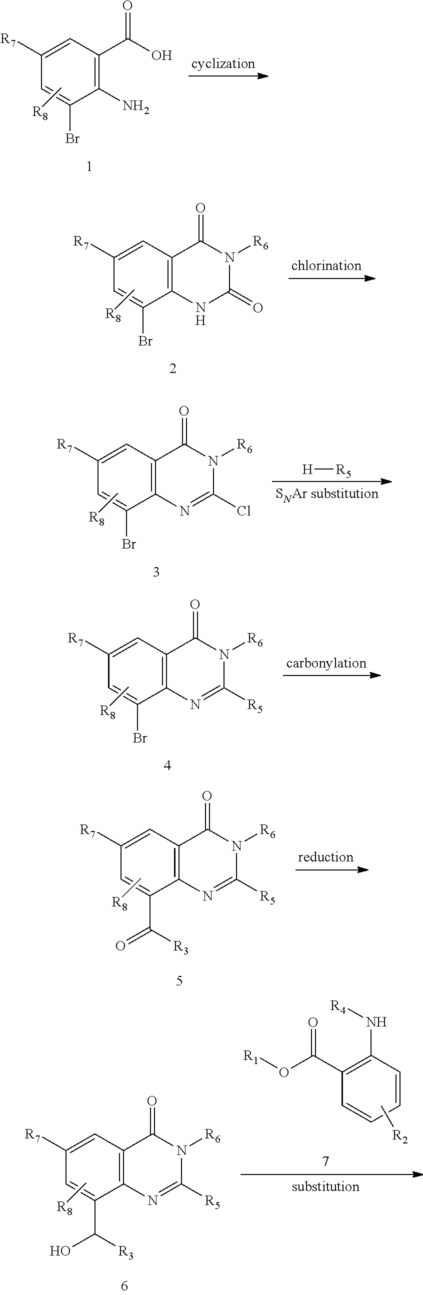

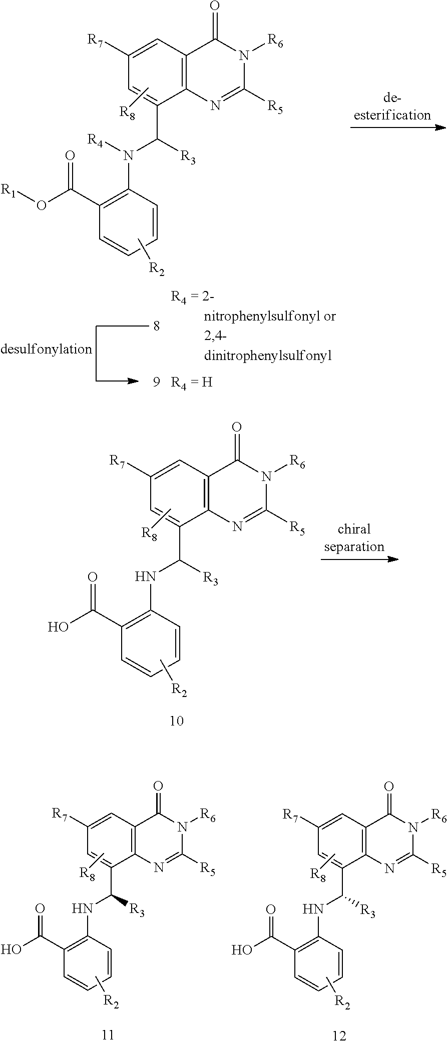

Beginning from aminobenzoic acids 1 that are generally known or available commercially (such as from BLD Pharmatech Ltd.), intermediate quinazoline-2,4-diones 2 can be generated under cyclization conditions. In an exemplary embodiment when R₆=H, cyclization occurred by treatment with urea at elevated temperatures. In exemplary embodiments when R₆=alkyl, cyclization occurred via a 2-step process consisting first of amide coupling with the corresponding alkylamine and HATU, followed by treatment with triphosgene. Chlorination of 2 furnished intermediates of general structure 3. In an exemplary embodiment, chlorination occurred by refluxing 2 in POCl₃ followed by neutralization with aqueous base (NaOH when R₆=H, or alternatively NaHCO₃ when R₆=alkyl). Intermediates of general structure 4 were generated by S_{N}Ar substitution. In exemplary embodiments, treatment of 3 with an amine, an amine hydrochloride salt, an alcohol, or a thiol (depending on whether R₅ is desired to be an N-link, O-link, or S-link, respectively), in some embodiments with the addition of an appropriate base (e.g., but not limited to, DIEA, K₂CO₃, or NaH), in an appropriate solvent (e.g., but not limited to, ACN, NMP, or DMF) at room temperature or, in some embodiments, at elevated temperature (up to 140° C.), yielded intermediates of general structure 4. Intermediates of general structure 5 were generated by carbonylation. In some exemplary embodiments, the bromine substituent of 4 was replaced with an acetyl group by treatment with tributyl(1-ethoxyvinyl)tin and a catalytic palladium species (e.g., but not limited to, Pd(PPh₃)₄ or PdCl₂(PPh₃)₂) at elevated temperature, followed by hydrolysis with aqueous HCl, to yield ketones 5. Intermediates 5 when R₃=H can also be generated by formylation of 4 through a variety of known methods (e.g., but not limited to, palladium-catalyzed carbonylation in the presence of H₂ (Klaus, et al., Angew. Chem. Int. Ed. 2006, 45, 154), or cyanation followed by DIBAL reduction). Intermediates 5 can serve as a platform for further expansion into diverse R₃ substitutions (e.g., trifluoromethyl, difluoromethyl, fluoromethyl, alkyls, etc.) through a variety of known techniques (including but not limited to Prakash, et al., J. Am. Chem. Soc. 1989, 111, 393; Zhao, et al., Org. Lett. 2011, 13, 5342; Reichel, et al., Angew. Chem. Int. Ed. 2020, 59, 12268; etc.). Alcohol intermediates of general structure 6 can be prepared by reduction of 5. In an exemplary embodiment, the reduction was performed by treatment of 5 with NaBH₄ in MeOH. Conversion of 6 to arylamines of general structures 8 and 9 can be accomplished via substitution reactions. In some exemplary embodiments, 6 was converted to 9 via Mitsunobu conditions including PPh₃, DBAD, and intermediate 7 when R₄=H. In other exemplary embodiments, substitution of 6 under Mitsunobu conditions with intermediates 7 when R₄=2-nitrophenylsulfonyl or 2,4-dinitrophenylsulfonyl yielded sulfonamide intermediates 8. In other exemplary embodiments, 6 was converted first to the methanesulfonate via treatment with Ms₂O or MsCl and an amine base (e.g., but not limited to, Et₃N or DIEA), followed by treatment with amines 7, to furnish benzyl amines 9. Sulfonamides of general structure 8 may be converted to secondary amines 9 via known desulfonylation protocols. In an exemplary embodiment, treatment of 8 with potassium thiophenolate and K₂CO₃ yielded intermediate 9. Intermediates 9 with R₅=thioalkyl or thio(hetero)aryl may be further expanded to the corresponding sulfinyl or sulfonyl compounds via known oxidation methods (e.g., but not limited to, treatment with mCPBA or potassium peroxymonosulfate). Ester intermediates 9 can be converted to the corresponding carboxylic acids of general structure 10 via known de-esterification protocols. In some exemplary embodiments when R₁=CH₃, de-esterification was achieved via treatment with a metal hydroxide (e.g., but not limited to, LiOH or NaOH), yielding products of general structure 10. In other exemplary embodiments, intermediates 9 when R₁=tert-butyl were de-esterified by treatment with an appropriate acid (e.g., but not limited to, TFA, or HCl in 1,4-dioxane or water) to yield 10. Separation of racemic 10 via known chiral HPLC chromatographic techniques (e.g., but not limited to, use of DAICEL Chiralpak columns) yielded enantiomerically enriched compounds of general structures 11 and 12.

The following compounds (in their racemic or enantiomerically resolved forms) represent various embodiments of the present invention. The listing of substituents within brackets for a given compound indicates individual compounds containing one of each of the substituents.

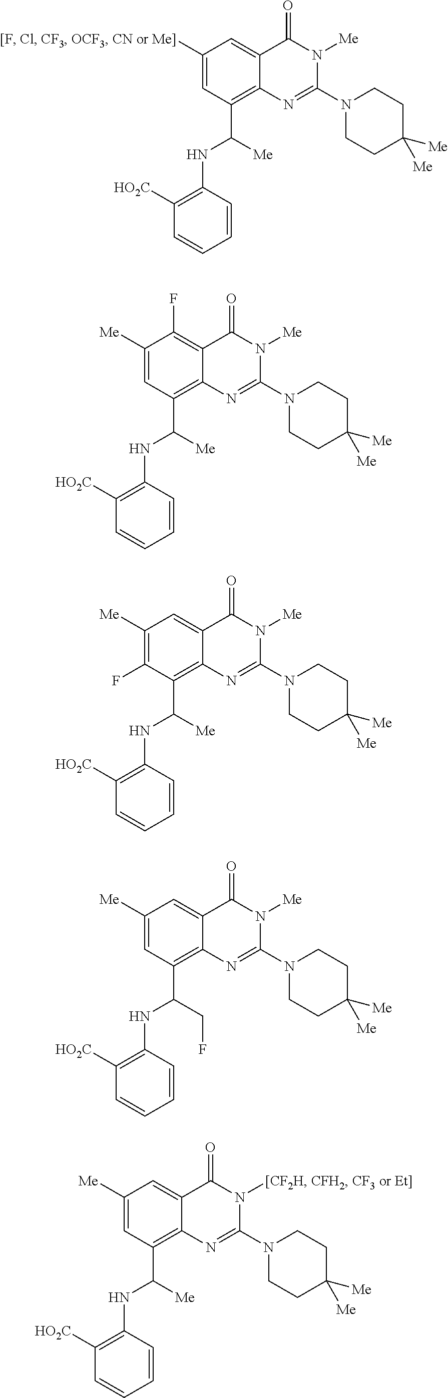

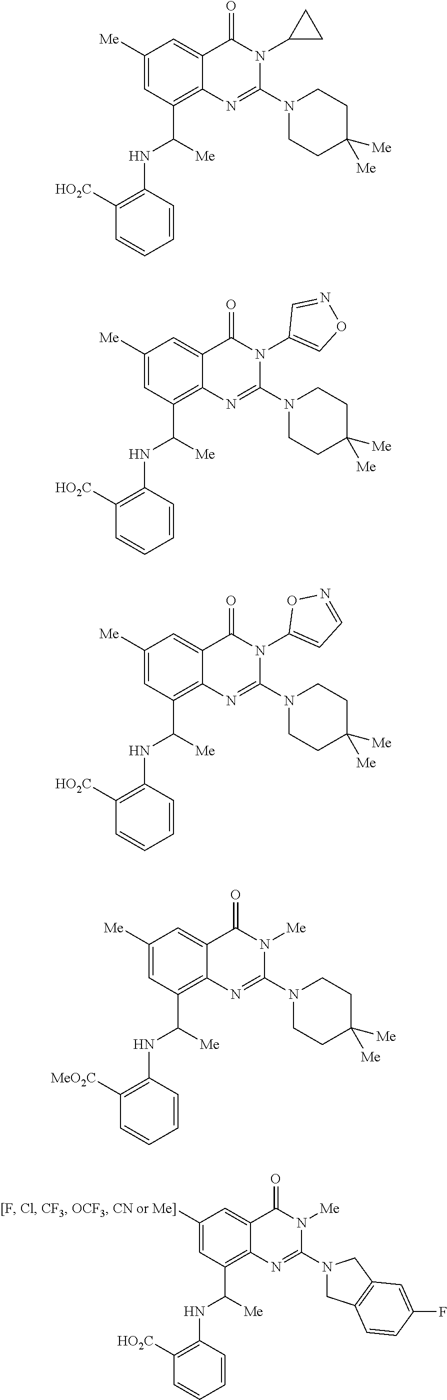

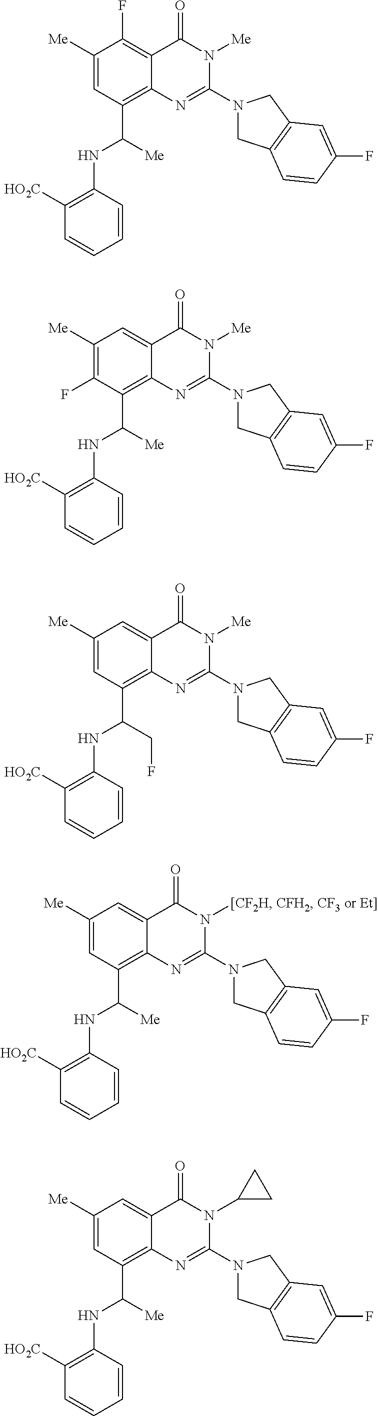

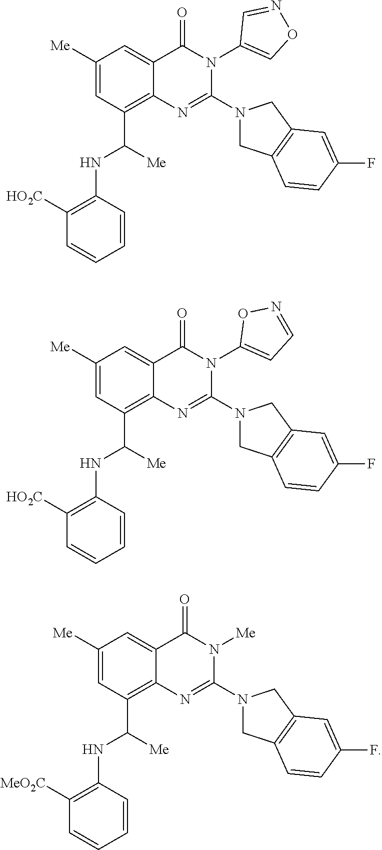 [SMILES: Cc1cc(C(C)Nc2ccccc2C(=O)O)c2nc(N3Cc4ccc(F)cc4C3)n(-c3cnoc3)c(=O)c2c1 ; Cc1cc(C(C)Nc2ccccc2C(=O)O)c2nc(N3Cc4ccc(F)cc4C3)n(-c3ccno3)c(=O)c2c1 ; COC(=O)c1ccccc1NC(C)c1cc(C)cc2c(=O)n(C)c(N3Cc4ccc(F)cc4C3)nc12]

The following compounds represent various embodiments of the present invention where only R₅ has been varied in a structure where all other atoms have been set. The carbon atom of the below generic structure marked with * is a chiral center and exists as a (R)- and (S)-racemic mixture or as either (R)- or (S)-enantiomer. The listing of substituents within brackets for a given compound indicates individual compounds containing one of each of the substituents. Where present, each Rₕ and Rᵢ is independently selected from H, CH₃, c-Pr, c-Bu, CF₃ and OH; and each Rⱼ is independently selected from CF₃, CH₂CF₃, CH₂CF₂H, OCH₃, OCF₃, OCH₂CF₃, Oc-Pr, aryl, heteroaryl, COCH₃ and CO₂CH₃.

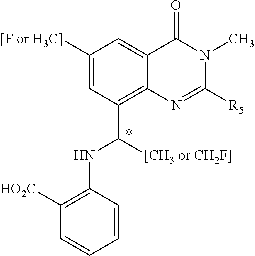

where R₅ is selected from

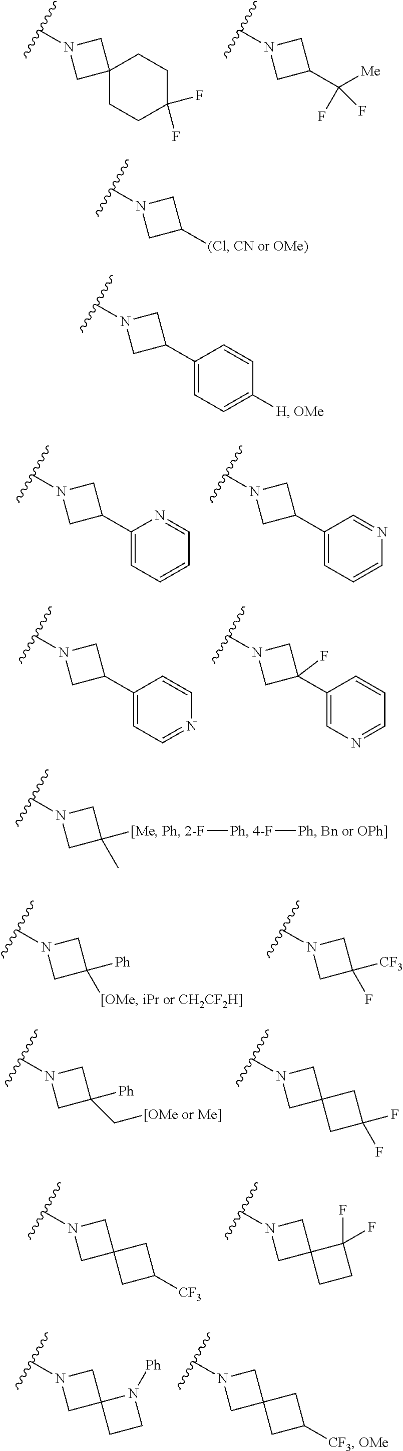

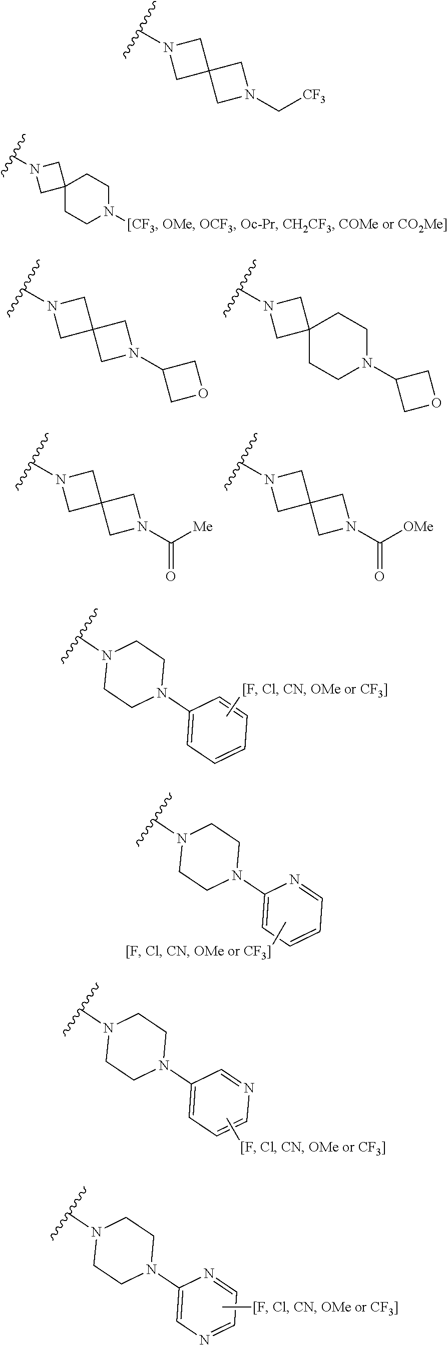

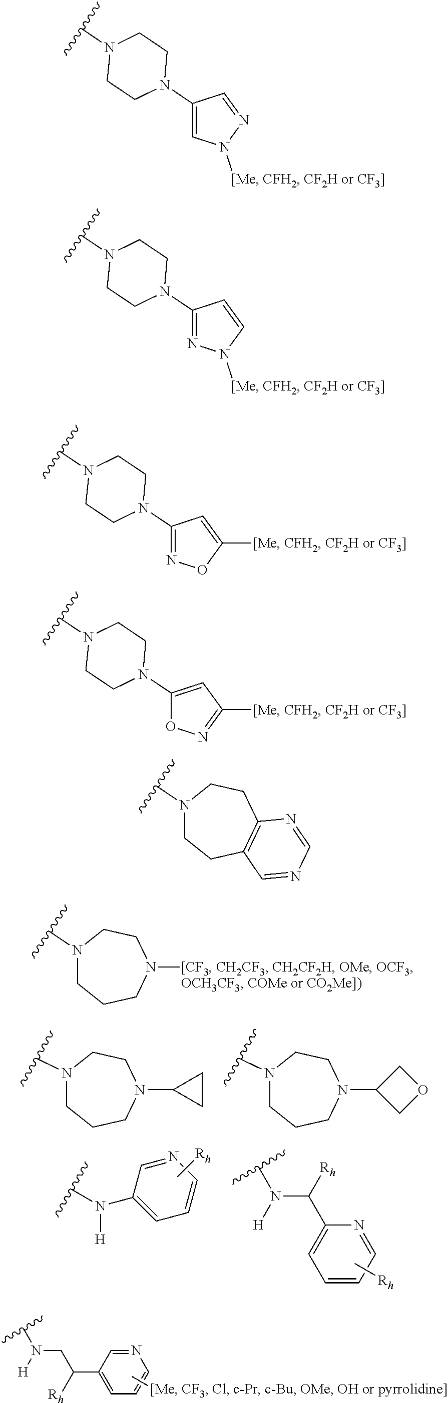

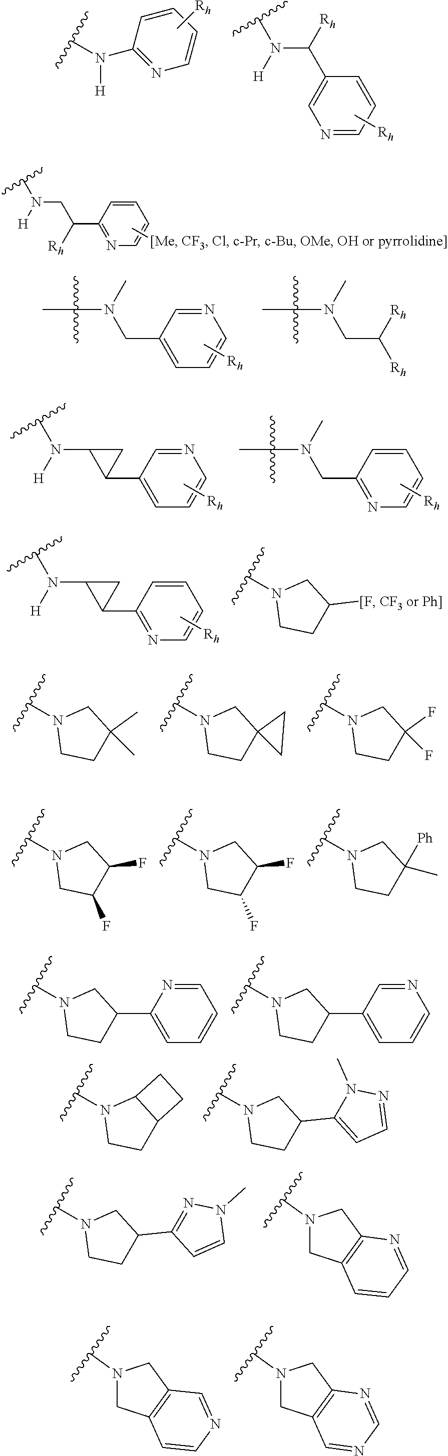

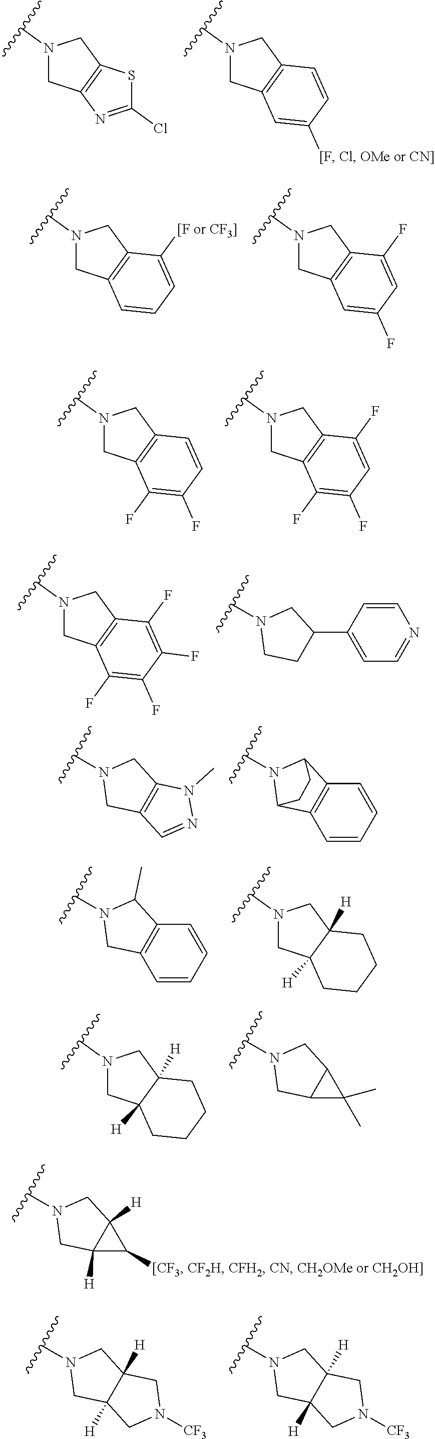

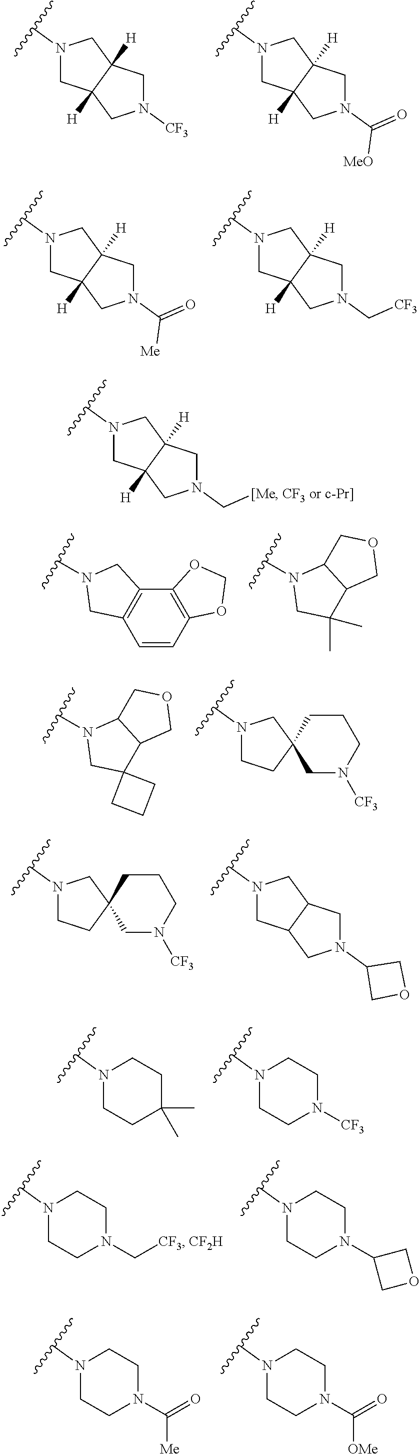

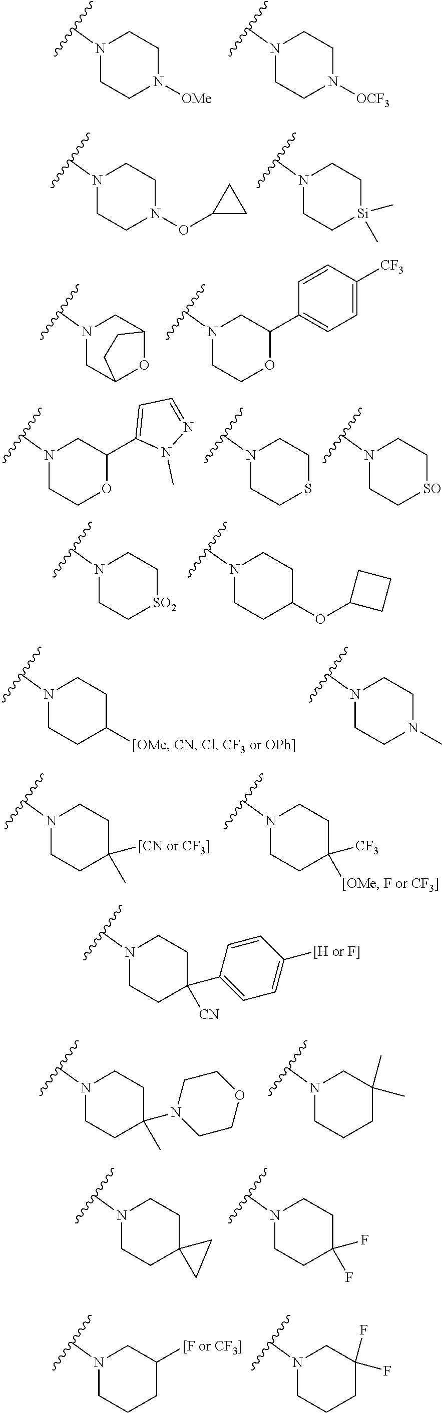

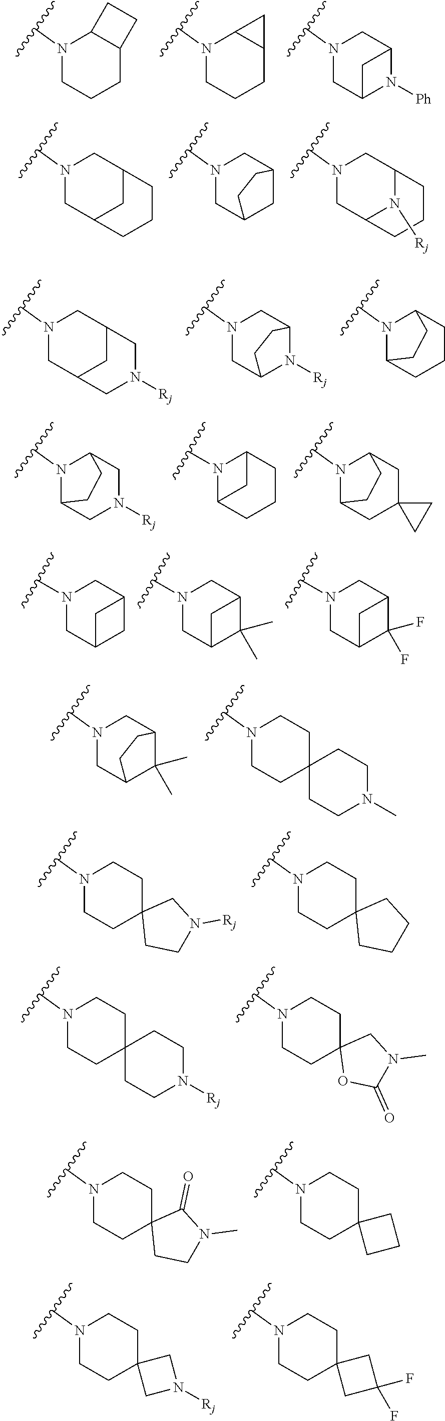

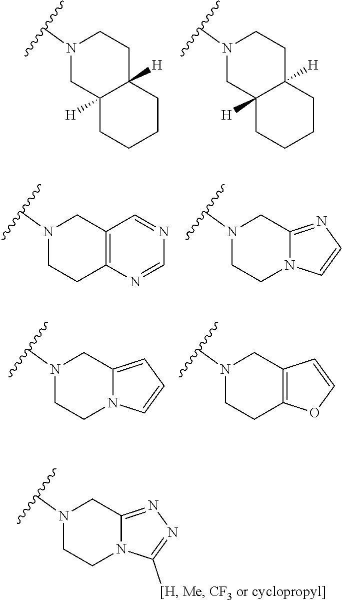

The following compounds represent various embodiments of the present invention where only R₅ has been varied in a structure where all other atoms have been set. The carbon atom in the below generic structure marked with * is a chiral center and exists as a (R)- and (S)-racemic mixture or as either (R)- or (S)-enantiomer. The listing of substituents within brackets for a given compound indicates individual compounds containing one of each of the substituents.

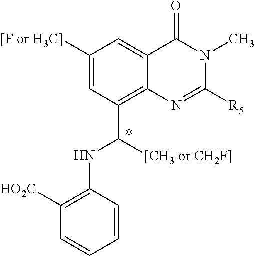

where R₅ is

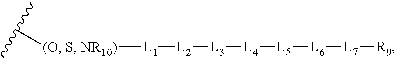

where L₁ to L₇, R₁₀ and R₉ are as defined herein, and where present, each Rₖ and Rₘ is independently selected from H, C₁-C₃ alkyl and acetyl (C(O)CH₃). In exemplary embodiments, R₅ is selected from

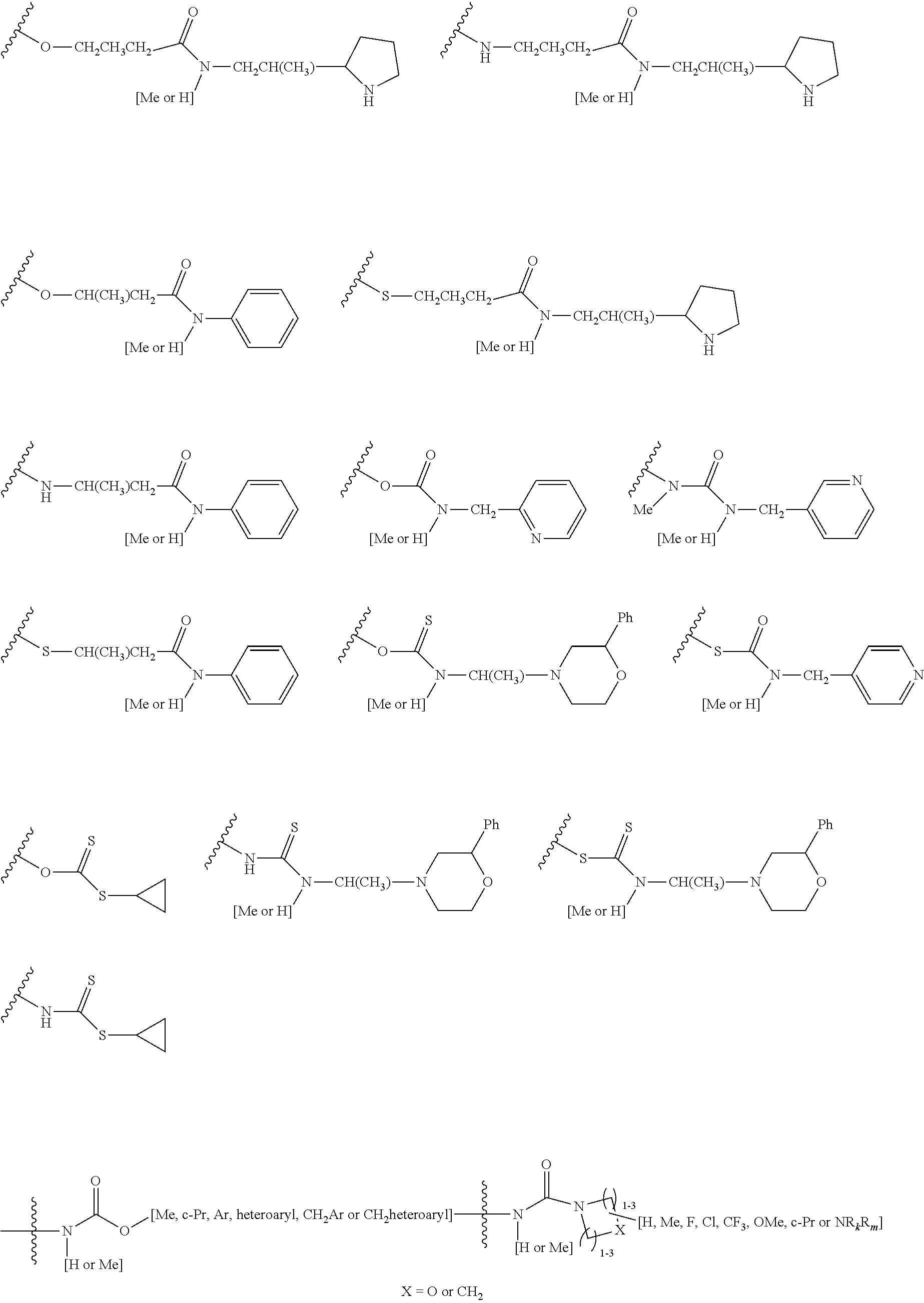

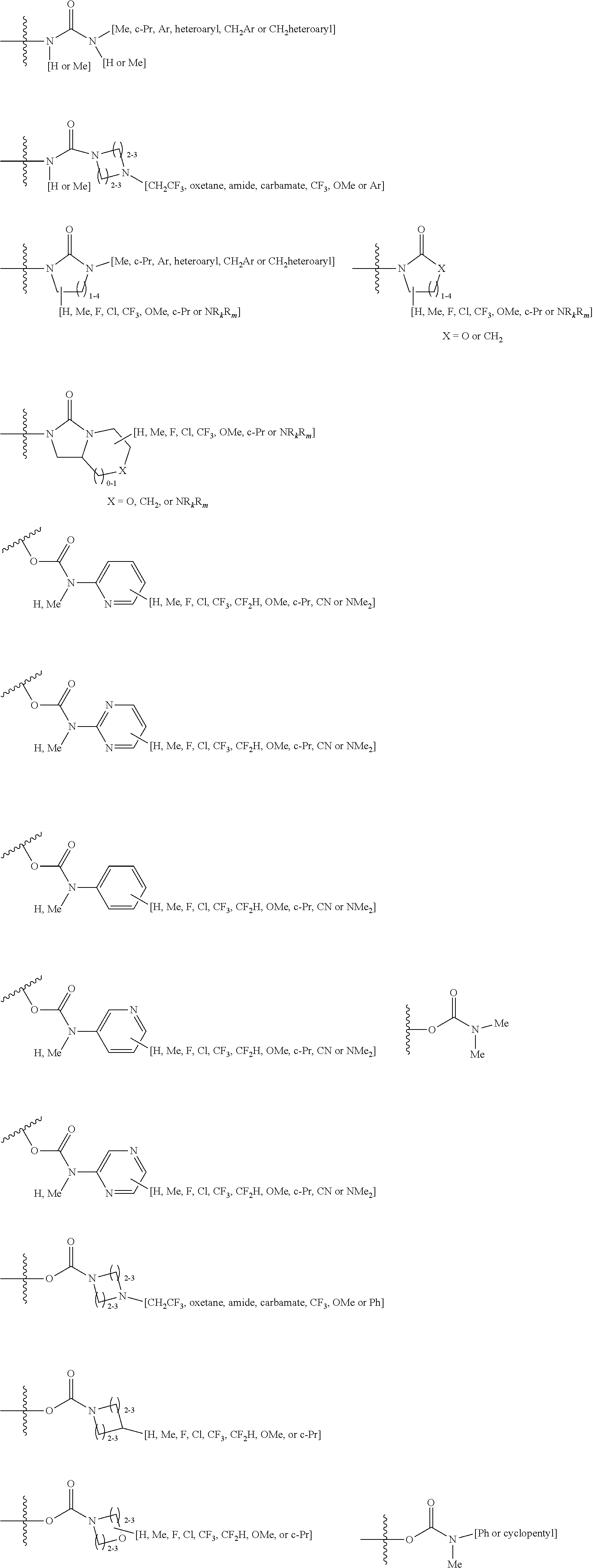

The following compounds represent various embodiments of the present invention where only R₅ has been varied in a structure where all other atoms have been set. The carbon atom of the below generic structure marked with * is a chiral center and exists as a (R)- and (S)-racemic mixture or as either (R)- or (S)-enantiomer. The listing of substituents within brackets for a given compound indicates individual compounds containing one of each of the substituents. Variable “A” is selected from O, S, S(O) and S(O)₂.

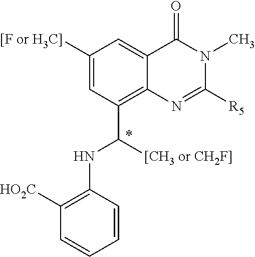

where R₅ is

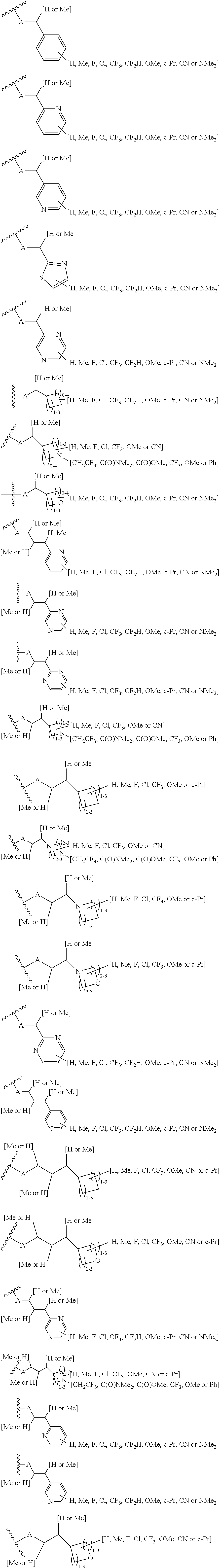

### EXPERIMENTAL

All commercially available solvents and reagents were used as received. All ¹H NMR spectra were recorded using a Bruker Avance III HD 300 MHz or Bruker Avance III HD 400 MHz. MS samples were analyzed on a SHIM-ADZU LCMS-2020 mass spectrometer with electrospray ionization operating in positive and negative ion mode. Samples were introduced into the mass spectrometer using chromatography. All final products had a purity of ≥90%, unless specified otherwise in the experimental details. HPLC purity was measured on a SHIM-ADZU Acquity HPLC system.

The following represents acronyms used in the experimental section for well-known chemical solvents, reagents, parameters and techniques:
¹H-NMR: proton nuclear magnetic resonance spectroscopy
ACN: acetonitrile
AcOH: acetic acid
c-Bu: cyclobutyl
c-Pr: cyclopropyl
DBAD: di-tert-butyl azodicarboxylate
DCM: dichloromethane
DIBAL: diisobutylaluminum hydride
DIEA: N,N-diisopropylethylamine
DMF: N,N-dimethylformamide
DMSO: dimethyl sulfoxide
ee: enantiomeric excess
Et₃N: triethylamine
EtOAc: ethyl acetate
EtOH: ethanol
FA: formic acid
h: hours
HATU: 1-[Bis(dimethylamino)methylene]-1H-1,2,3-triazolo[4,5-b]pyridinium 3-oxide hexafluorophosphate
HCl: hydrochloric acid
HPLC: high-performance liquid chromatography
IPA: isopropanol
K₂CO₃: potassium carbonate
LiOH: lithium hydroxide
mCPBA: meta-chloroperoxybenzoic acid
Me: methyl
MeOH: methanol
mg: milligram
min: minutes
mL: milliliter
MsCl: methanesulfonyl chloride
Ms₂O: methanesulfonic anhydride
NaBH₄: sodium borohydride
NaH: sodium hydride
NaOH: sodium hydroxide
NaHCO₃: sodium bicarbonate
Na₂SO₄: sodium sulfate
NMP: N-methylpyrrolidone
oxetane: 4-membered ring containing 3 carbon ring atoms and 1 oxygen ring atom.
Pd(PPh₃)₄: tetrakis(triphenylphosphine)palladium(0)
PdCl₂(PPh₃)₂ bis(triphenylphosphine)palladium(II) dichloride
PE: petroleum ether
POCl₃: phosphorus oxychloride
PPh₃: triphenylphosphine
TFA: trifluoroacetic acid
THF: tetrahydrofuran
TLC: thin-layer chromatography

### EXAMPLES

### Intermediate 1: 8-bromo-2-chloro-3,6-dimethylquinazolin-4-one

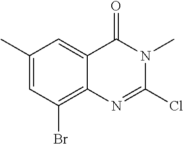

### Step 1: Preparation of 2-amino-3-bromo-N,5-dimethylbenzamide

To a stirred solution of commercially available (e.g., BLD Pharmatech Ltd.) 2-amino-3-bromo-5-methylbenzoic acid (50 g, 0.22 mmol) and DIEA (227 mL, 1.30 mol) in DCM (100 mL) and DMF (3.36 mL) was added HATU (165 g, 435 mmol). After stirring for 5 min at room temperature, methylamine hydrochloride (66 g, 978 mmol) was added. The resulting solution was allowed to stir for 2 h at room temperature. The mixture was concentrated under reduced pressure and purified by silica gel chromatography (0-50% EtOAc in PE) to yield the desired product 2-amino-3-bromo-N,5-dimethylbenzamide (50 g, 95% yield) as a solid. MS: (ES⁺) m/z=243.0 [M+H]⁺.

### Step 2: Preparation of 8-bromo-3,6-dimethyl-1H-quinazoline-2,4-dione

A mixture of 2-amino-3-bromo-N,5-dimethylbenzamide (19.2 g, 79 mmol), triphosgene (23.4 g, 79 mmol), and DIEA (13.8 mL, 79 mmol) in DCM (250 mL) was stirred overnight at 50° C. under a nitrogen atmosphere. The mixture was allowed to cool to room temperature and then was concentrated under reduced pressure. The crude product was purified by silica gel chromatography, eluted with 0-50% EtOAc in PE, to afford the title compound 8-bromo-3,6-dimethyl-1H-quinazoline-2,4-dione (18 g, 85% yield) as a solid. MS: (ES⁺) m/z=269.0 [M+H]⁺.

### Step 3: Preparation of 8-bromo-2-chloro-3,6-dimethylquinazolin-4-one

A mixture of 8-bromo-3,6-dimethyl-1H-quinazoline-2,4-dione (2.9 g, 10.78 mmol) and DIEA (7.51 mL, 43.11 mmol) in POCl₃ (13.2 mL, 141.4 mmol) was stirred overnight at 100° C. under a nitrogen atmosphere. The mixture was concentrated under reduced pressure and quenched with water (100 mL), then neutralized to pH=7 with NaHCO₃. The resulting solution was extracted with DCM (3×30 mL) and the combined organic layers were washed with brine (20 mL), dried over Na₂SO₄ and concentrated under reduced pressure. The crude product was purified by silica gel chromatography, eluted with 0-50% EtOAc in PE, to yield 8-bromo-2-chloro-3,6-dimethylquinazolin-4-one (800 mg, 26% yield) as a solid. ¹H NMR (chloroform-d, 300 MHz): δ 7.90 (dd, J=2.0, 0.9 Hz, 1H), 7.85-7.79 (m, 1H), 3.73 (s, 3H), 2.43 (s, 3H), MS: (ES⁺) m/z=287.0 [M+H]⁺.

### Intermediate 2: tert-butyl 2-(2-nitrobenzenesulfonamido) benzoate

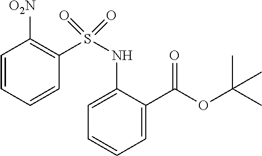 [SMILES: CC(C)(C)OC(=O)c1ccccc1NS(=O)(=O)c1ccccc1[N+](=O)[O-]]

A stirred solution of tert-butyl 2-aminobenzoate (5.00 g, 25.87 mmol) in DCM (100 mL) and pyridine (10 mL) was cooled to 0° C. in an ice bath, then treated with a solution of 2-nitrobenzenesulfonyl chloride (11.47 g, 51.74 mmol) in DCM (20 mL) dropwise. The resulting mixture was stirred overnight at room temperature, then diluted with water (50 mL) and extracted with DCM (3×50 mL). The combined organic layers were washed with brine (2×50 mL), dried over anhydrous Na₂SO₄, filtered, and concentrated under reduced pressure. The crude product was purified by silica gel chromatography, eluted with 0-33% EtOAc in PE over 30 min, to isolate tert-butyl 2-(2-nitrobenzenesulfonamido)benzoate (5.20 g, 53% yield) as a solid. ¹H NMR (DMSO-d₆, 400 MHz): δ 11.01 (s, 1H), 8.19 (dd, J=7.7, 1.5 Hz, 1H), 8.07 (dd, J=7.8, 1.4 Hz, 1H), 8.00-7.80 (m, 3H), 7.64-7.53 (m, 2H), 7.21 (ddd, J=8.2, 6.7, 1.9 Hz, 1H), 1.55 (s, 9H), MS (ES⁻) m/z=376.8 [M−H]⁻.

### Intermediate 3: methyl 2-(2,4-dinitrobenzenesulfonamido)benzoate

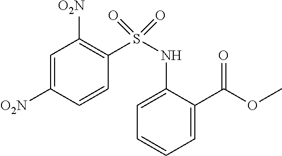 [SMILES: COC(=O)c1ccccc1NS(=O)(=O)c1ccc([N+](=O)[O-])cc1[N+](=O)[O-]]

A solution of methyl anthranilate (5 g, 33.08 mmol) in DCM (50 mL) and pyridine (5.23 g, 66.15 mmol) in a 250 mL round-bottom flask was cooled to 0° C. in an ice bath, then 2,4-dinitrobenzenesulfonyl chloride (10.58 g, 39.70 mmol) was added. The resulting mixture was stirred overnight while warming to room temperature under a nitrogen atmosphere, then diluted with water (50 mL) and extracted with DCM (3×50 mL). The combined organic phases were washed with brine (50 mL), dried with Na₂SO₄, filtered, and concentrated under reduced pressure. The residue was purified by silica gel column chromatography, eluted with PE:EtOAc=1:1, to afford methyl 2-(2,4-dinitrobenzenesulfonamido)benzoate (8 g, 63% yield) as a solid. ¹H NMR (chloroform-d, 300 MHz): δ 11.42 (s, 1H), 8.63 (d, J=2.2 Hz, 1H), 8.49 (dd, J=8.6, 2.2 Hz, 1H), 8.38 (d, J=8.6 Hz, 1H), 8.02 (dd, J=8.0, 1.7 Hz, 1H), 7.81 (d, J=8.4 Hz, 1H), 7.54 (td, J=8.3, 7.9, 1.7 Hz, 1H), 7.21-7.09 (m, 1H), 3.96 (s, 3H), MS: (ES⁻) m/z=380.0 [M−H]⁻.

### Intermediates 4, 5, 6 and 7

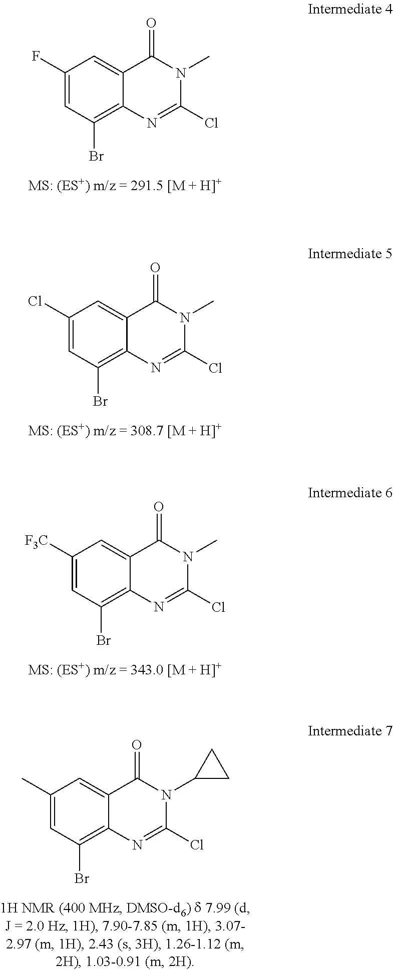

Intermediates 4, 5, 6 and 7 were prepared in a similar manner to Intermediate 1.

### Example 1: 2-((1-(2-(4,4-dimethyl-1,4-azasilinan-1-yl)-3,6-dimethyl-4-oxo-3,4-dihydroquinazolin-8-yl)ethyl)amino)benzoic acid (Enantiomer 1); and

### Example 2: 2-((1-(2-(4,4-dimethyl-1,4-azasilinan-1-yl)-3,6-dimethyl-4-oxo-3,4-dihydroquinazolin-8-yl)ethyl)amino)benzoic acid (Enantiomer 2)

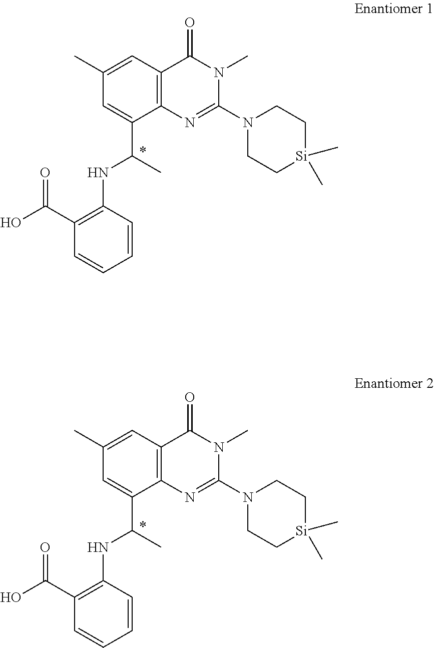 [SMILES: Cc1cc(C(C)Nc2ccccc2C(=O)O)c2nc(N3CC[Si](C)(C)CC3)n(C)c(=O)c2c1 ; Cc1cc(C(C)Nc2ccccc2C(=O)O)c2nc(N3CC[Si](C)(C)CC3)n(C)c(=O)c2c1]

### Step 1: Preparation of 8-bromo-2-(4,4-dimethyl-1,4-azasilinan-1-yl)-3,6-dimethylquinazolin-4-one

A solution of 8-bromo-2-chloro-3,6-dimethylquinazolin-4-one (Intermediate 1) (1.20 g, 4.18 mmol) and 4,4-dimethyl-1,4-azasilinane (450 mg, 3.48 mmol) in ACN (10 mL) was stirred overnight at 80° C. The reaction mixture was concentrated under reduced pressure and the residue was purified by silica gel column chromatography, eluted with PE:EtOAc=5:1, to afford 8-bromo-2-(4,4-dimethyl-1,4-azasilinan-1-yl)-3,6-dimethylquinazolin-4-one (800 mg, 60% yield) as a solid. MS: (ES⁺) m/z=380.1 [M+H]⁺.

### Step 2: Preparation of 8-acetyl-2-(4,4-dimethyl-1,4-azasilinan-1-yl)-3,6-dimethylquinazolin-4-one

A solution of 8-bromo-2-(4,4-dimethyl-1,4-azasilinan-1-yl)-3,6-dimethylquinazolin-4-one (800 mg, 2.1 mmol) and tributyl(1-ethoxyvinyl)tin (836 mg, 2.3 mmol) at room temperature under a nitrogen atmosphere, followed by the addition of Pd(PPh₃)₄ (266 mg, 0.23 mmol) in dioxane (10 mL) stirred for overnight at 100° C. The mixture was allowed to cool down to room temperature. To the above mixture was added 1 M HCl (2 mL). The resulting mixture was stirred for 1 h at 50° C. The resulting mixture was concentrated under vacuum. The residue was purified by Prep-TLC (PE/EA=1:1) to afford 8-acetyl-2-(4,4-dimethyl-1,4-azasilinan-1-yl)-3,6-dimethylquinazolin-4-one (600 mg, 83% yield) as a light yellow solid. MS: (ES⁺) m/z=344.2 [M+H]⁺.

### Step 3: Preparation of 2-(4,4-dimethyl-1,4-azasilinan-1-yl)-8-(1-hydroxyethyl)-3,6-dimethylquinazolin-4-one

A solution of 8-acetyl-2-(4,4-dimethyl-1,4-azasilinan-1-yl)-3,6-dimethylquinazolin-4-one (600 mg, 1.74 mmol) and NaBH₄ (264 mg, 6.98 mmol) in MeOH (5 mL) was stirred for 2 h at 0° C. The resulting mixture was concentrated under vacuum. The residue was purified by Prep-TLC (PE/EA=1:1) to afford 2-(4,4-dimethyl-1,4-azasilinan-1-yl)-8-(1-hydroxyethyl)-3,6-dimethylquinazolin-4-one (400 mg, 66% yield) as a white solid. MS: (ES⁺) m/z=346.6 [M+H]⁺.

### Step 4: Preparation of tert-butyl 2-((N-(1-(2-(4,4-dimethyl-1,4-azasilinan-1-yl)-3,6-dimethyl-4-oxo-3,4-dihydroquinazolin-8-yl)ethyl)-2-nitrophenyl)sulfonamido)benzoate

A stirred solution of 2-(4,4-dimethyl-1,4-azasilinan-1-yl)-8-(1-hydroxyethyl)-3,6-dimethylquinazolin-4-one (400 mg, 1.15 mmol), PPh₃ (759 mg, 2.89 mmol), and tert-butyl 2-(2-nitrobenzenesulfonamido) benzoate (Intermediate 2) (657 mg, 1.73 mmol) in THE (5 mL) was cooled to 0° C., then DBAD (800 mg, 3.47 mmol) in THE (5 mL) was added dropwise. The resulting mixture was stirred overnight at room temperature under a nitrogen atmosphere, then quenched with water (5 mL) and extracted with EtOAc (3×10 mL). The combined organic layers were washed with brine (5 mL), dried over Na₂SO₄, filtered, and concentrated under reduced pressure. The resulting residue was purified by preparative TLC, eluted with DCM:MeOH=20:1, to afford tert-butyl 2-((N-(1-(2-(4,4-dimethyl-1,4-azasilinan-1-yl)-3,6-dimethyl-4-oxo-3,4-dihydroquinazolin-8-yl)ethyl)-2-nitrophenyl)sulfonamido)benzoate (500 mg, 61% yield) as a solid. MS: (ES⁺) m/z=706.3 [M+H]⁺.

### Step 5: Preparation of tert-butyl 2-((1-(2-(4,4-dimethyl-1,4-azasilinan-1-yl)-3,6-dimethyl-4-oxo-3,4-dihydroquinazolin-8-yl)ethyl)amino)benzoate

A mixture of tert-butyl 2-((N-(1-(2-(4,4-dimethyl-1,4-azasilinan-1-yl)-3,6-dimethyl-4-oxo-3,4-dihydroquinazolin-8-yl)ethyl)-2-nitrophenyl)sulfonamido)benzoate (300 mg, 0.42 mmol), K₂CO₃ (174 mg, 1.3 mmol), and potassium thiophenolate (252 mg, 1.7 mmol) in DMF (10 mL) was stirred for 3 h at 60° C. The solution was cooled to room temperature, diluted with water (20 mL), and extracted with EtOAc (3×10 mL). The organic layers were combined, washed with water (2×10 mL) and dried over anhydrous MgSO₄. After filtration, the filtrate was concentrated under reduced pressure and purified by preparative TLC, eluted with PE:EtOAc=2:1, to afford tert-butyl 2-((1-(2-(4,4-dimethyl-1,4-azasilinan-1-yl)-3,6-dimethyl-4-oxo-3,4-dihydroquinazolin-8-yl)ethyl)amino)benzoate (180 mg, 81% yield) as a solid. MS: (ES⁺) m/z=521.2 [M+H]⁺.

### Step 6: Preparation of 2-((1-(2-(4,4-dimethyl-1,4-azasilinan-1-yl)-3,6-dimethyl-4-oxo-3,4-dihydroquinazolin-8-yl)ethyl)amino)benzoic acid

A solution of tert-butyl 2-((1-(2-(4,4-dimethyl-1,4-azasilinan-1-yl)-3,6-dimethyl-4-oxo-3,4-dihydroquinazolin-8-yl)ethyl)amino)benzoate (180 mg, 0.35 mmol) and HCl (4 M in 1,4-dioxane, 5 mL) was stirred overnight at room temperature. The resulting mixture was concentrated under reduced pressure and purified by preparative TLC, eluted with DCM:MeOH=10:1, to afford 2-((1-(2-(4,4-dimethyl-1,4-azasilinan-1-yl)-3,6-dimethyl-4-oxo-3,4-dihydroquinazolin-8-yl)ethyl)amino)benzoic acid (100 mg, 62% yield) as a solid. MS: (ES⁺) m/z=465.3 [M+H]⁺.

### Step 7: Preparation of Example 1 2-((1-(2-(4,4-dimethyl-1,4-azasilinan-1-yl)-3,6-dimethyl-4-oxo-3,4-dihydroquinazolin-8-yl)ethyl)amino)benzoic acid, Enantiomer 1; and Example 2 2-((1-(2-(4,4-dimethyl-1,4-azasilinan-1-yl)-3,6-dimethyl-4-oxo-3,4-dihydroquinazolin-8-yl)ethyl)amino)benzoic acid, Enantiomer 2

The racemic mixture of 2-((1-(2-(4,4-dimethyl-1,4-azasilinan-1-yl)-3,6-dimethyl-4-oxo-3,4-dihydroquinazolin-8-yl)ethyl)amino)benzoic acid (100 mg, 0.22 mmol) was separated by chiral preparative HPLC (column: CHIRALPAK IF, 2×25 cm, 5 μm; mobile phase: hexanes (0.1% FA):EtOH=95:5; flow rate: 20 mL/min) to furnish the title compounds Example 1: 2-((1-(2-(4,4-dimethyl-1,4-azasilinan-1-yl)-3,6-dimethyl-4-oxo-3,4-dihydroquinazolin-8-yl)ethyl)amino)benzoic acid, Enantiomer 1 (34.6 mg, 39% yield, >99% ee), ¹H NMR (DMSO-d₆, 300 MHz): δ 12.63 (s, 1H), 8.43 (s, 1H), 7.76 (d, J=7.9 Hz, 1H), 7.68 (s, 1H), 7.43 (s, 1H), 7.21-7.09 (m, 1H), 6.52-6.36 (m, 2H), 5.46-5.31 (m, 1H), 3.54-3.39 (m, 7H), 2.30 (s, 3H), 1.53 (d, J=6.6 Hz, 3H), 1.02-0.79 (m, 4H), 0.12 (s, 6H), MS: (ES⁻) m/z=463.2 [M−H]⁻; and Example 2: 2-((1-(2-(4,4-dimethyl-1,4-azasilinan-1-yl)-3,6-dimethyl-4-oxo-3,4-dihydroquinazolin-8-yl)ethyl)amino)benzoic acid, Enantiomer 2 (21 mg, 23% yield, >99% ee), ¹H NMR (DMSO-d₆, 300 MHz): δ 12.60 (s, 1H), 8.43 (s, 1H), 7.76 (d, J=7.9 Hz, 1H), 7.68 (s, 1H), 7.43 (s, 1H), 7.20-7.09 (m, 1H), 6.51-6.37 (m, 2H), 5.46-5.31 (m, 1H), 3.53-3.40 (m, 7H), 2.30 (s, 3H), 1.53 (d, J=6.6 Hz, 3H), 1.00-0.81 (m, 4H), 0.12 (s, 6H), MS: (ES⁻) m/z=463.2 [M−H]⁻.

### Example 3: 2-((1-(2-(3,3-dimethylpiperidin-1-yl)-3,6-dimethyl-4-oxo-3,4-dihydroquinazolin-8-yl)ethyl)amino)benzoic acid (Enantiomer 1); and

### Example 4: 2-((1-(2-(3,3-dimethylpiperidin-1-yl)-3,6-dimethyl-4-oxo-3,4-dihydroquinazolin-8-yl)ethyl)amino)benzoic acid (Enantiomer 2)

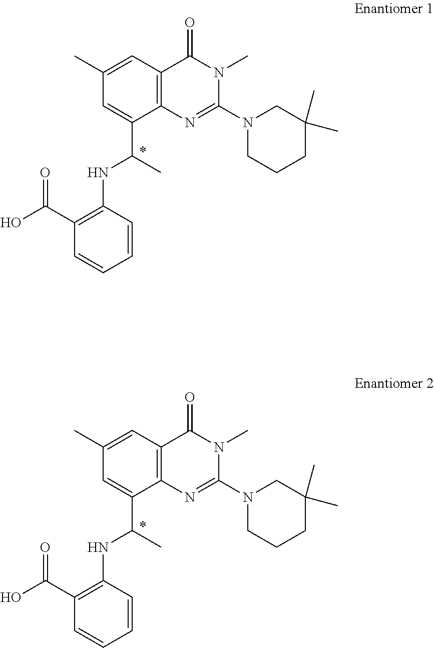 [SMILES: Cc1cc(C(C)Nc2ccccc2C(=O)O)c2nc(N3CCCC(C)(C)C3)n(C)c(=O)c2c1 ; Cc1cc(C(C)Nc2ccccc2C(=O)O)c2nc(N3CCCC(C)(C)C3)n(C)c(=O)c2c1]

### Step 1: Preparation of 8-bromo-2-(3,3-dimethylpiperidin-1-yl)-3,6-dimethylquinazolin-4-one

A mixture of 8-bromo-2-chloro-3,6-dimethylquinazolin-4-one (Intermediate 1) (700 mg, 2.43 mmol), 3,3-dimethylpiperidine hydrochloride (437 mg, 2.92 mmol) and DIEA (629 mg, 4.87 mmol) in ACN (7 mL) was stirred overnight at 100° C. under a nitrogen atmosphere. The resulting mixture was diluted with water (20 mL) and extracted with DCM (3×20 mL). The combined organic layers were washed with brine (20 mL), dried over anhydrous Na₂SO₄, filtered, and concentrated under reduced pressure. The residue was purified by silica gel column chromatography, eluted with PE:EtOAc=2:1, to afford 8-bromo-2-(3,3-dimethylpiperidin-1-yl)-3,6-dimethylquinazolin-4-one (700 mg, 79% yield). MS: (ES⁺) m/z=364.1 [M+H]⁺.

### Step 2: Preparation of 8-acetyl-2-(3,3-dimethylpiperidin-1-yl)-3,6-dimethylquinazolin-4-one

This step was performed in a manner similar to Example 1, Step 2, with PdCl₂(PPh₃)₂ being used in place of Pd(PPh₃)₄ as the catalyst, to afford 8-acetyl-2-(3,3-dimethylpiperidin-1-yl)-3,6-dimethylquinazolin-4-one (600 mg, 95% yield). MS: (ES⁺) m/z=328.1 [M+H]⁺.

### Step 3: Preparation of 2-(3,3-dimethylpiperidin-1-yl)-8-(1-hydroxyethyl)-3,6-dimethylquinazolin-4-one

This step was performed in a manner similar to Example 1, Step 3 to furnish 2-(3,3-dimethylpiperidin-1-yl)-8-(1-hydroxyethyl)-3,6-dimethylquinazolin-4-one (350 mg, 86% yield). MS: (ES⁺) m/z=330.1 [M+H]⁺.

### Step 4: Preparation of methyl 2-((1-(2-(3,3-dimethylpiperidin-1-yl)-3,6-dimethyl-4-oxo-3,4-dihydroquinazolin-8-yl)ethyl)amino)benzoate

A mixture of 2-(3,3-dimethylpiperidin-1-yl)-8-(1-hydroxyethyl)-3,6-dimethylquinazolin-4-one (600 mg, 1.82 mmol), methyl 2-(2,4-dinitrobenzenesulfonamido)benzoate (Intermediate 3) (1.04 g, 2.73 mmol), and PPh₃ (717 mg, 2.73 mmol) in THE (6 mL) was cooled to 0° C., then DBAD (629 mg, 2.73 mmol) was added. The resulting mixture was stirred for two days at room temperature under a nitrogen atmosphere. The 2,4-dinitrobenzenesulfonyl group was concomitantly removed during the course of the reaction. The reaction was diluted with water (20 mL) and extracted with DCM (3×30 mL). The combined organic layers were washed with brine (20 mL), dried over anhydrous Na₂SO₄, filtered, and concentrated under reduced pressure. The residue was purified by preparative TLC, eluted with PE:EtOAc=5:2, to afford methyl 2-((1-(2-(3,3-dimethylpiperidin-1-yl)-3,6-dimethyl-4-oxo-3,4-dihydroquinazolin-8-yl)ethyl)amino)benzoate (600 mg, 71% yield). MS: (ES⁺) m/z=463.3 [M+H]⁺.

### Step 5: Preparation of Example 3 2-((1-(2-(3,3-dimethylpiperidin-1-yl)-3,6-dimethyl-4-oxo-3,4-dihydroquinazolin-8-yl)ethyl)amino)benzoic acid, Enantiomer 1; and Example 4 2-((1-(2-(3,3-dimethylpiperidin-1-yl)-3,6-dimethyl-4-oxo-3,4-dihydroquinazolin-8-yl)ethyl)amino)benzoic acid, Enantiomer 2

A solution of methyl 2-((1-(2-(3,3-dimethylpiperidin-1-yl)-3,6-dimethyl-4-oxo-3,4-dihydroquinazolin-8-yl)ethyl)amino)benzoate (540 mg, 1.16 mmol) in THE (6 mL) was treated with a solution of LiOH (1.40 g, 58.35 mmol) in water (6 mL) at room temperature. The resulting mixture was stirred for two days at 90° C. under a nitrogen atmosphere. The mixture was acidified to pH=3 with 6 M aqueous HCl and the resulting mixture was extracted with DCM (5×50 mL). The combined organic layers were washed with water (50 mL), dried over anhydrous Na₂SO₄, filtered, and concentrated under reduced pressure. The residue was purified by reverse-phase flash chromatography (column: C18 silica gel; mobile phase: 60-100% ACN in water over 20 min). The racemic product was separated by chiral preparative HPLC (column: CHIRALPAK IF, 2×25 cm, 5 μm; mobile phase: hexanes (0.1% FA):EtOH=95:5; flow rate: 20 mL/min) to furnish title compounds Example 3: 2-((1-(2-(3,3-dimethylpiperidin-1-yl)-3,6-dimethyl-4-oxo-3,4-dihydroquinazolin-8-yl)ethyl)amino)benzoic acid, Enantiomer 1 (57 mg, 11% yield, >99% ee), ¹H NMR (methanol-d₄, 300 MHz): δ 7.87 (d, J=8.0 Hz, 1H), 7.78 (s, 1H), 7.50 (s, 1H), 7.16-7.02 (m, 1H), 6.55-6.39 (m, 2H), 5.61-5.43 (m, 1H), 3.61 (s, 3H), 3.27-3.10 (m, 2H), 2.96 (s, 2H), 2.34 (s, 3H), 1.94-1.76 (m, 2H), 1.60 (d, J=6.7 Hz, 3H), 1.54-1.43 (m, 2H), 1.08 (s, 3H), 1.07 (s, 3H), MS: (ES⁺) m/z=449.2 [M+H]⁺; and Example 4: 2-((1-(2-(3,3-dimethylpiperidin-1-yl)-3,6-dimethyl-4-oxo-3,4-dihydroquinazolin-8-yl)ethyl)amino)benzoic acid, Enantiomer 2 (65 mg, 12% yield, 98% ee), ¹H NMR (methanol-d₄, 300 MHz): δ 7.87 (d, J=8.0 Hz, 1H), 7.78 (s, 1H), 7.50 (s, 1H), 7.18-7.00 (m, 1H), 6.55-6.39 (m, 2H), 5.57-5.44 (m, 1H), 3.61 (s, 3H), 3.27-3.11 (m, 2H), 2.96 (s, 2H), 2.34 (s, 3H), 1.94-1.76 (m, 2H), 1.60 (d, J=6.8 Hz, 3H), 1.54-1.41 (m, 2H), 1.08 (s, 3H), 1.07 (s, 3H), MS: (ES⁺) m/z=449.2 [M+H]⁺.

### Example 5: 2-((1-(3,6-dimethyl-4-oxo-2-(2-azaspiro[3.5]nonan-2-yl)-3,4-dihydroquinazolin-8-yl)ethyl)amino)benzoic acid, Enantiomer 1; and

### Example 6: 2-((1-(3,6-dimethyl-4-oxo-2-(2-azaspiro[3.5]nonan-2-yl)-3,4-dihydroquinazolin-8-yl)ethyl)amino)benzoic acid, Enantiomer 2

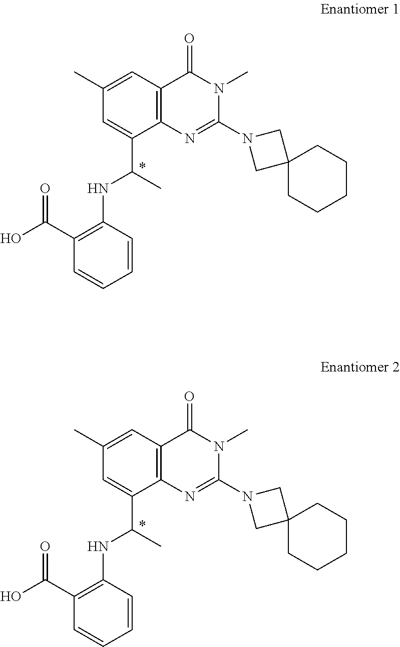 [SMILES: Cc1cc(C(C)Nc2ccccc2C(=O)O)c2nc(N3CC4(CCCCC4)C3)n(C)c(=O)c2c1 ; Cc1cc(C(C)Nc2ccccc2C(=O)O)c2nc(N3CC4(CCCCC4)C3)n(C)c(=O)c2c1]

### Step 1: Preparation of 8-bromo-3,6-dimethyl-2-(2-azaspiro[3.5]nonan-2-yl)quinazolin-4(3H)-one

Performed in a manner similar to Example 1, Step 1 using 2-azaspiro[3.5]nonane as the amine to afford 8-bromo-3,6-dimethyl-2-(2-azaspiro[3.5]nonan-2-yl)quinazolin-4(3H)-one (1.35 g, 52% yield) as a solid. MS: (ES⁺) m/z=376.1 [M+H]⁺.

### Step 2: Preparation of 8-acetyl-3,6-dimethyl-2-(2-azaspiro[3.5]nonan-2-yl)quinazolin-4(3H)-one

This step was performed in a manner similar to Example 1, Step 2 to afford 8-acetyl-3,6-dimethyl-2-(2-azaspiro[3.5]nonan-2-yl)quinazolin-4(3H)-one (1 g, 82% yield) as a solid. MS: (ES⁺) m/z=340.2 [M+H]⁺.

### Step 3: Preparation of 8-(1-hydroxyethyl)-3,6-dimethyl-2-(2-azaspiro[3.5]nonan-2-yl)quinazolin-4(3H)-one

This step was performed in a manner similar to Example 1, Step 3 to afford 8-(1-hydroxyethyl)-3,6-dimethyl-2-(2-azaspiro[3.5]nonan-2-yl)quinazolin-4(3H)-one (450 mg, 44% yield) as a solid. MS: (ES⁺) m/z=342.1 [M+H]⁺.

### Step 4: Preparation of tert-butyl 2-((N-(1-(3,6-dimethyl-4-oxo-2-(2-azaspiro[3.5]nonan-2-yl)-3,4-dihydroquinazolin-8-yl)ethyl)-2-nitrophenyl)sulfonamido)benzoate

Performed in a manner similar to Example 1, Step 4 to afford tert-butyl 2-((N-(1-(3,6-dimethyl-4-oxo-2-(2-azaspiro[3.5]nonan-2-yl)-3,4-dihydroquinazolin-8-yl)ethyl)-2-nitrophenyl)sulfonamido)benzoate (625 mg, 76% yield) as a solid. MS: (ES⁺) m/z=702.3 [M+H]⁺.

### Step 5: Preparation of tert-butyl 2-((1-(3,6-dimethyl-4-oxo-2-(2-azaspiro[3.5]nonan-2-yl)-3,4-dihydroquinazolin-8-yl)ethyl)amino)benzoate

Performed in a manner similar to Example 1, Step 5 to afford tert-butyl 2-((1-(3,6-dimethyl-4-oxo-2-(2-azaspiro[3.5]nonan-2-yl)-3,4-dihydroquinazolin-8-yl)ethyl)amino)benzoate (200 mg, 53% yield) as a solid. MS: (ES⁺) m/z=517.3 [M+H]⁺.

### Step 6: Preparation of 2-((1-(3,6-dimethyl-4-oxo-2-(2-azaspiro[3.5]nonan-2-yl)-3,4-dihydroquinazolin-8-yl)ethyl)amino)benzoic acid

Performed in a manner similar to Example 1, Step 6 to afford 2-((1-(3,6-dimethyl-4-oxo-2-(2-azaspiro[3.5]nonan-2-yl)-3,4-dihydroquinazolin-8-yl)ethyl)amino)benzoic acid (110 mg, 62% yield) as a solid. MS: (ES⁺) m/z=461.2 [M+H]⁺.

### Step 7: Preparation of Example 5 2-((1-(3,6-dimethyl-4-oxo-2-(2-azaspiro[3.5]nonan-2-yl)-3,4-dihydroquinazolin-8-yl)ethyl)amino)benzoic acid, Enantiomer 1; and Example 6 (2-((1-(3,6-dimethyl-4-oxo-2-(2-azaspiro[3.5]nonan-2-yl)-3,4-dihydroquinazolin-8-yl)ethyl)amino)benzoic acid, Enantiomer 2

The racemic mixture of 2-((1-(3,6-dimethyl-4-oxo-2-(2-azaspiro[3.5]nonan-2-yl)-3,4-dihydroquinazolin-8-yl)ethyl)amino)benzoic acid (110 mg, 0.24 mmol) was purified by chiral preparative HPLC (column: CHIRAL ART Cellulose-SB, 2×25 cm, 5 μm; mobile phase: hexanes (0.1% FA):EtOH=93:7; Flow rate: 20 mL/min). The relevant fractions were concentrated to furnish the title compounds Example 5: 2-((1-(3,6-dimethyl-4-oxo-2-(2-azaspiro[3.5]nonan-2-yl)-3,4-dihydroquinazolin-8-yl)ethyl)amino)benzoic acid, Enantiomer 1 (8.0 mg, 8% yield, >99% ee), ¹H NMR (DMSO-d₆, 300 MHz): δ 8.44 (s, 1H), 7.85-7.74 (m, 1H), 7.65 (s, 1H), 7.40 (s, 1H), 7.23-7.10 (m, 1H), 6.56-6.41 (m, 2H), 5.46-5.28 (m, 1H), 3.94 (s, 4H), 3.40 (s, 3H), 2.28 (s, 3H), 1.79-1.60 (m, 4H), 1.54 (d, J=6.6 Hz, 3H), 1.50-1.19 (m, 6H), MS: (ES⁺) m/z=461.2 [M+H]⁺; and Example 6: (2-((1-(3,6-dimethyl-4-oxo-2-(2-azaspiro[3.5]nonan-2-yl)-3,4-dihydroquinazolin-8-yl)ethyl)amino)benzoic acid, Enantiomer 2 (10.2 mg, 10% yield, >99% ee), ¹H NMR (DMSO-d₆, 300 MHz): δ 8.43 (s, 1H), 8.00-7.73 (m, 1H), 7.65 (s, 1H), 7.40 (s, 1H), 7.32-7.05 (m, 1H), 6.66-6.34 (m, 2H), 5.55-5.23 (m, 1H), 3.95 (s, 4H), 3.40 (s, 3H), 2.29 (s, 3H), 1.81-1.60 (m, 4H), 1.55 (d, J=6.5 Hz, 3H), 1.50-1.13 (m, 6H), MS: (ES⁺) m/z=461.1 [M+H]⁺.

### Example 7: 2-((1-(2-(5-methoxyisoindolin-2-yl)-3,6-dimethyl-4-oxo-3,4-dihydroquinazolin-8-yl)ethyl)amino)benzoic acid, Enantiomer 1; and

### Example 8: 2-((1-(2-(5-methoxyisoindolin-2-yl)-3,6-dimethyl-4-oxo-3,4-dihydroquinazolin-8-yl)ethyl)amino)benzoic acid, Enantiomer 2

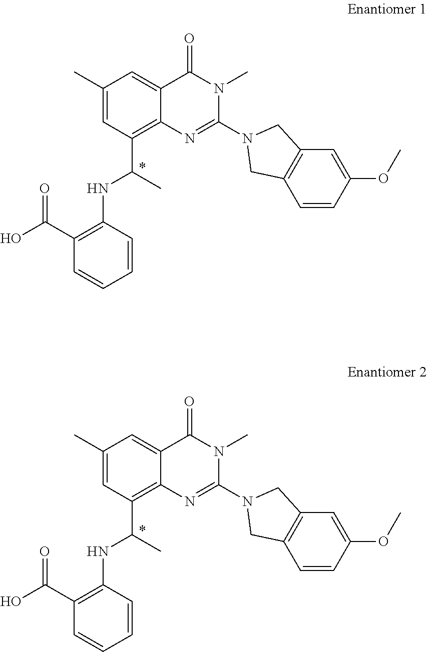 [SMILES: COc1ccc2c(c1)CN(c1nc3c(C(C)Nc4ccccc4C(=O)O)cc(C)cc3c(=O)n1C)C2 ; COc1ccc2c(c1)CN(c1nc3c(C(C)Nc4ccccc4C(=O)O)cc(C)cc3c(=O)n1C)C2]

### Step 1: Preparation of 8-bromo-2-(5-methoxy-1,3-dihydroisoindol-2-yl)-3,6-dimethylquinazolin-4-one

A mixture of 8-bromo-2-chloro-3,6-dimethylquinazolin-4-one (Intermediate 1) (900 mg, 3.13 mmol), 5-methoxy-2,3-dihydro-1H-isoindole (514 mg, 3.44 mmol), and K₂CO₃ (865 mg, 6.26 mmol) in NMP (20 mL) was stirred at 120° C. overnight. The resulting mixture was cooled to room temperature, diluted with water (10 mL), and extracted with DCM (3×10 mL). The combined organic layers were washed with brine (3×10 mL), dried over anhydrous Na₂SO₄, filtered, and concentrated under reduced pressure. The residue was purified by silica gel column chromatography, eluted with PE:EtOAc=9:1, to afford 8-bromo-2-(5-methoxy-1,3-dihydroisoindol-2-yl)-3,6-dimethylquinazolin-4-one (900 mg, 72% yield) as a solid. MS: (ES⁺) m/z=400.0 [M+H]⁺.

### Step 2: Preparation of 8-acetyl-2-(5-methoxy-1,3-dihydroisoindol-2-yl)-3,6-dimethylquinazolin-4-one

This step was performed in a manner similar to Example 1, Step 2, with PdCl₂(PPh₃)₂ used as the catalyst instead of Pd(PPh₃)₄, to afford 8-acetyl-2-(5-methoxy-1,3-dihydroisoindol-2-yl)-3,6-dimethylquinazolin-4-one (560 mg, 62% yield) as a solid. MS: (ES⁺) m/z=364.2 [M+H]⁺.

### Step 3: Preparation of 8-(1-hydroxyethyl)-2-(5-methoxy-1,3-dihydroisoindol-2-yl)-3,6-dimethylquinazolin-4-one

A mixture of 8-acetyl-2-(5-methoxy-1,3-dihydroisoindol-2-yl)-3,6-dimethylquinazolin-4-one (500 mg, 1.38 mmol) and MeOH (5 mL) was cooled to 0° C. and treated with NaBH₄ (260 mg, 6.88 mmol). The mixture was heated to 80° C. and stirred overnight. The resulting mixture was cooled to room temperature and diluted with water (30 mL), then extracted with DCM (3×10 mL). The combined organic layers were washed with brine (3×10 mL), dried over anhydrous Na₂SO₄, filtered, and concentrated under reduced pressure. The crude product was purified by reverse-phase flash chromatography (C18 silica gel; 10% to 80% MeOH in water over 40 min) to afford 8-(1-hydroxyethyl)-2-(5-methoxy-1,3-dihydroisoindol-2-yl)-3,6-dimethylquinazolin-4-one (200 mg, 40% yield) as a solid. MS: (ES⁺) m/z=366.2 [M+H]⁺.

### Step 4: Preparation of methyl 2-((1-(2-(5-methoxyisoindolin-2-yl)-3,6-dimethyl-4-oxo-3,4-dihydroquinazolin-8-yl)ethyl)amino)benzoate

Into a 40 mL vial were added 8-(1-hydroxyethyl)-2-(5-methoxy-1,3-dihydroisoindol-2-yl)-3,6-dimethylquinazolin-4-one (200 mg, 0.547 mmol), THE (5 mL), methyl anthranilate (124 mg, 0.82 mmol) and PPh₃ (359 mg, 1.37 mmol). To the mixture was added DBAD (315 mg, 1.37 mmol) in THE (5 mL) at 0° C., and the mixture was stirred at room temperature for overnight under N₂. The resulting mixture was diluted with water (10 mL). The resulting mixture was extracted with CH₂Cl₂ (3×10 mL). The combined organic layers were washed with brine (3×10 mL) and dried over anhydrous Na₂SO₄. After filtration, the filtrate was concentrated under reduced pressure. The residue was purified by Prep-TLC (PE:EA=3:1) to afford a crude product. The crude product was purified by reverse phase flash with the following conditions to afford methyl 2-((1-(2-(5-methoxyisoindolin-2-yl)-3,6-dimethyl-4-oxo-3,4-dihydroquinazolin-8-yl)ethyl)amino)benzoate (40 mg, 15% yield) as a white solid. MS: (ES⁺) m/z=499.2 [M+H]⁺.

### Step 5: Preparation of Example 11 2-((1-(2-(5-methoxyisoindolin-2-yl)-3,6-dimethyl-4-oxo-3,4-dihydroquinazolin-8-yl)ethyl)amino)benzoic acid, Enantiomer 1; and Example 12 2-((1-(2-(5-methoxyisoindolin-2-yl)-3,6-dimethyl-4-oxo-3,4-dihydroquinazolin-8-yl)ethyl)amino)benzoic acid, Enantiomer 2

A mixture of methyl 2-((1-(2-(5-methoxyisoindolin-2-yl)-3,6-dimethyl-4-oxo-3,4-dihydroquinazolin-8-yl)ethyl)amino)benzoate (60 mg, 0.12 mmol) and LiOH hydrate (101 mg, 2.40 mmol) in THE (1 mL) and water (1 mL) was stirred at room temperature overnight. The mixture was acidified to pH=6 with 1 M aqueous HCl and extracted with DCM (3×10 mL). The combined organic layers were washed with brine (3×10 mL), dried over anhydrous Na₂SO₄, filtered, and concentrated under reduced pressure. The residue was purified by reverse-phase flash chromatography (column: C18 silica gel; mobile phase: 5-100% ACN in water over 40 min) to afford the racemic product, which was purified further by chiral preparative HPLC (column: CHIRALPAK IF, 2×25 cm, 5 μm; mobile phase: hexanes (0.1% FA):EtOH=9:1; flow rate: 20 mL/min)) to afford the title compounds Example 7: 2-((1-(2-(5-methoxyisoindolin-2-yl)-3,6-dimethyl-4-oxo-3,4-dihydroquinazolin-8-yl)ethyl)amino)benzoic acid, Enantiomer 1 (5.8 mg, 10% yield, 98% ee), ¹H NMR (methanol-d₄, 300 MHz): δ 7.87 (dd, J=8.0, 1.7 Hz, 1H), 7.76 (dd, J=2.1, 1.0 Hz, 1H), 7.48 (d, J=2.1 Hz, 1H), 7.24 (d, J=8.3 Hz, 1H), 7.06 (ddd, J=8.7, 7.1, 1.7 Hz, 1H), 6.93 (d, J=2.3 Hz, 1H), 6.87 (dd, J=8.3, 2.4 Hz, 1H), 6.52-6.39 (m, 2H), 5.50 (q, J=6.6 Hz, 1H), 5.03 (td, J=19.0, 18.6, 14.7 Hz, 4H), 3.80 (s, 3H), 3.70 (s, 3H), 2.32 (s, 3H), 1.61 (d, J=6.7 Hz, 3H), MS: (ES⁺) m/z=485.2 [M+H]⁺; and Example 8: 2-((1-(2-(5-methoxyisoindolin-2-yl)-3,6-dimethyl-4-oxo-3,4-dihydroquinazolin-8-yl)ethyl)amino)benzoic acid, Enantiomer 2 (6.8 mg, 11% yield, 99% ee), ¹H NMR (methanol-d₄, 300 MHz): δ 7.87 (dd, J=8.0, 1.7 Hz, 1H), 7.76 (dd, J=2.1, 1.0 Hz, 1H), 7.48 (d, J=2.1 Hz, 1H), 7.24 (d, J=8.3 Hz, 1H), 7.05 (ddd, J=8.7, 7.1, 1.7 Hz, 1H), 6.92 (d, J=2.3 Hz, 1H), 6.87 (dd, J=8.3, 2.4 Hz, 1H), 6.52-6.39 (m, 2H), 5.50 (q, J=6.6 Hz, 1H), 5.04 (td, J=19.0, 18.6, 14.7 Hz, 4H), 3.79 (s, 3H), 3.69 (s, 3H), 2.32 (s, 3H), 1.61 (d, J=6.7 Hz, 3H), MS: (ES⁺) m/z=485.2 [M+H]⁺.

### Example 9: 2-((1-(2-(5-chloroisoindolin-2-yl)-3,6-dimethyl-4-oxo-3,4-dihydroquinazolin-8-yl)ethyl)amino)benzoic acid, Enantiomer 1; and

### Example 10: 2-((1-(2-(5-chloroisoindolin-2-yl)-3,6-dimethyl-4-oxo-3,4-dihydroquinazolin-8-yl)ethyl)amino)benzoic acid, Enantiomer 2

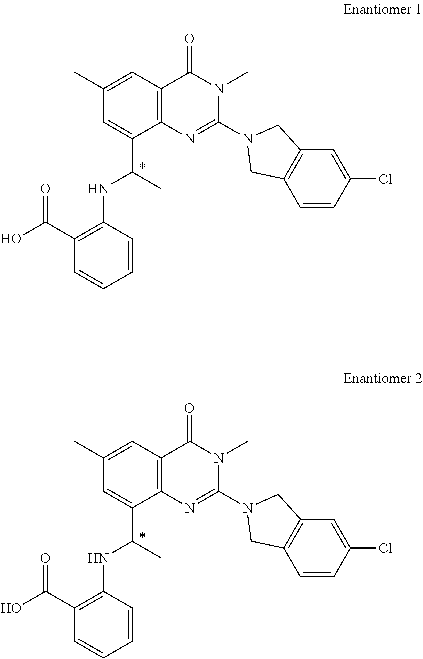 [SMILES: Cc1cc(C(C)Nc2ccccc2C(=O)O)c2nc(N3Cc4ccc(Cl)cc4C3)n(C)c(=O)c2c1 ; Cc1cc(C(C)Nc2ccccc2C(=O)O)c2nc(N3Cc4ccc(Cl)cc4C3)n(C)c(=O)c2c1]

### Step 1: Preparation of 8-bromo-2-(5-chloro-1,3-dihydroisoindol-2-yl)-3,6-dimethylquinazolin-4-one

Performed in a manner similar to Example 1, Step 1 using 5-chloro-2,3-dihydro-1H-isoindole hydrochloride as the amine to afford 8-bromo-2-(5-chloro-1,3-dihydroisoindol-2-yl)-3,6-dimethylquinazolin-4-one (2.8 g, 99% yield) as a solid. MS: (ES⁺) m/z=406.0 [M+H]⁺.

### Step 2: Preparation of 8-acetyl-2-(5-chloro-1,3-dihydroisoindol-2-yl)-3,6-dimethylquinazolin-4-one

This step was performed in a manner similar to Example 1, Step 2 to afford 8-acetyl-2-(5-chloro-1,3-dihydroisoindol-2-yl)-3,6-dimethylquinazolin-4-one (2 g, 78% yield) as a solid. MS: (ES⁺) m/z=368.1 [M+H]⁺.

### Step 3: Preparation of 2-(5-chloro-1,3-dihydroisoindol-2-yl)-8-(1-hydroxyethyl)-3,6-dimethylquinazolin-4-one

Performed in a manner similar to Example 1, Step 3 to afford 2-(5-chloro-1,3-dihydroisoindol-2-yl)-8-(1-hydroxyethyl)-3,6-dimethylquinazolin-4-one (483 mg, 24% yield) as a solid. MS: (ES⁺) m/z=370.1 [M+H]⁺.

### Step 4: Preparation of methyl 2-((1-(2-(5-chloroisoindolin-2-yl)-3,6-dimethyl-4-oxo-3,4-dihydroquinazolin-8-yl)ethyl)amino)benzoate

Performed in a manner similar to Example 1, Step 4 to furnish methyl 2-((1-(2-(5-chloroisoindolin-2-yl)-3,6-dimethyl-4-oxo-3,4-dihydroquinazolin-8-yl)ethyl)amino)benzoate (181 mg, 44% yield) as an oil. MS: (ES⁺) m/z=503.1 [M+H]⁺.

### Step 5: Preparation of Example 9 2-((1-(2-(5-chloroisoindolin-2-yl)-3,6-dimethyl-4-oxo-3,4-dihydroquinazolin-8-yl)ethyl)amino)benzoic acid, Enantiomer 1; and Example 10 2-((1-(2-(5-chloroisoindolin-2-yl)-3,6-dimethyl-4-oxo-3,4-dihydroquinazolin-8-yl)ethyl)amino)benzoic acid, Enantiomer 2

The preparation of racemic 2-((1-(2-(5-chloroisoindolin-2-yl)-3,6-dimethyl-4-oxo-3,4-dihydroquinazolin-8-yl)ethyl)amino)benzoic acid was performed in a manner similar to Example 1, Step 5, which was then separated by chiral preparative HPLC (column: CHIRALPAK IF, 2×25 cm, 5 μm; mobile phase: hexanes (0.1% FA):IPA=9:1; flow rate: 20 mL/min)) to afford the title compounds Example 9: 2-((1-(2-(5-chloroisoindolin-2-yl)-3,6-dimethyl-4-oxo-3,4-dihydroquinazolin-8-yl)ethyl)amino)benzoic acid, Enantiomer 1 (17 mg, 11% yield, >99% ee), ¹H NMR (methanol-d₄, 300 MHz): δ 7.86 (dd, J=7.9, 1.7 Hz, 1H), 7.76 (dd, J=2.0, 1.0 Hz, 1H), 7.48 (d, J=2.1 Hz, 1H), 7.39-7.23 (m, 3H), 7.03 (ddd, J=8.5, 7.1, 1.6 Hz, 1H), 6.50-6.35 (m, 2H), 5.47 (q, J=6.7 Hz, 1H), 5.14-4.91 (m, 4H), 3.67 (s, 3H), 2.32 (s, 3H), 1.59 (d, J=6.6 Hz, 3H), MS: (ES⁺) m/z=489.2 [M+H]⁺; and Example 10: 2-((1-(2-(5-chloroisoindolin-2-yl)-3,6-dimethyl-4-oxo-3,4-dihydroquinazolin-8-yl)ethyl)amino)benzoic acid, Enantiomer 2 (14 mg, 9% yield, 99% ee), ¹H NMR (methanol-d₄, 300 MHz): δ 7.86 (dd, J=7.9, 1.7 Hz, 1H), 7.76 (dd, J=2.0, 1.0 Hz, 1H), 7.48 (d, J=2.1 Hz, 1H), 7.40-7.21 (m, 3H), 7.04 (ddd, J=8.6, 7.1, 1.6 Hz, 1H), 6.51-6.34 (m, 2H), 5.48 (q, J=6.6 Hz, 1H), 5.11-4.91 (m, 4H), 3.68 (s, 3H), 2.32 (s, 3H), 1.59 (d, J=6.7 Hz, 3H), MS: (ES⁺) m/z=489.2 [M+H]⁺.

### Example 11: 2-((1-(2-isobutoxy-3,6-dimethyl-4-oxo-3,4-dihydroquinazolin-8-yl)ethyl)amino)benzoic acid, Enantiomer 1; and

### Example 12: 2-((1-(2-isobutoxy-3,6-dimethyl-4-oxo-3,4-dihydroquinazolin-8-yl)ethyl)amino)benzoic acid, Enantiomer 2

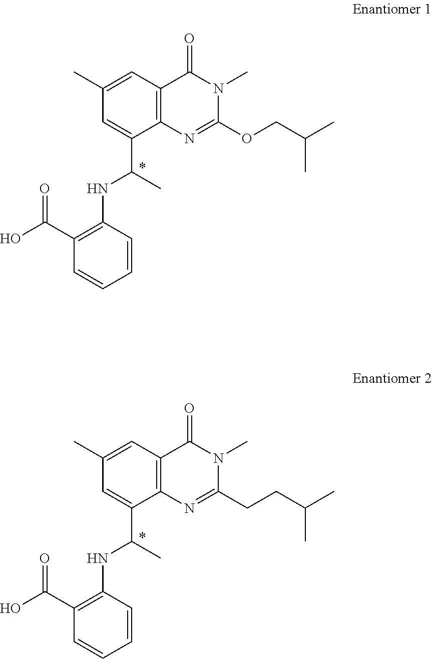 [SMILES: Cc1cc(C(C)Nc2ccccc2C(=O)O)c2nc(OCC(C)C)n(C)c(=O)c2c1 ; Cc1cc(C(C)Nc2ccccc2C(=O)O)c2nc(CCC(C)C)n(C)c(=O)c2c1]

### Step 1: Preparation of 8-bromo-2-isobutoxy-3,6-dimethylquinazolin-4(3H)-one

A solution of 8-bromo-2-chloro-3,6-dimethylquinazolin-4-one (Intermediate 1) (170 mg, 0.59 mmol) in DMF (6 mL) was treated with NaH (36 mg, 0.89 mmol) and stirred for 30 min at room temperature, then isobutanol (53 mg, 0.71 mmol) was added. The reaction was stirred at room temperature for 1 h, then quenched with water (20 mL) and extracted with EtOAc (3×20 mL). The combined organic phases were washed with brine (20 mL), dried over Na₂SO₄, filtered, and concentrated under reduced pressure. The crude residue was purified by silica gel chromatography, eluted with PE:EtOAc=10:1, to give the title product 8-bromo-2-isobutoxy-3,6-dimethylquinazolin-4(3H)-one as a solid (160 mg, 83% yield). MS: (ES⁺) m/z=325.0 [M+H]⁺.

### Step 2: Preparation of 8-acetyl-3,6-dimethyl-2-(2-methylpropoxy)quinazolin-4-one

Prepared in a manner similar to Example 1, Step 2 to yield the title product 8-acetyl-3,6-dimethyl-2-(2-methylpropoxy)quinazolin-4-one as a solid (120 mg, 71% yield). MS: (ES⁺) m/z=289.1 [M+H]⁺.

### Step 3: Preparation of 8-(1-hydroxyethyl)-3,6-dimethyl-2-(2-methylpropoxy)quinazolin-4-one

Prepared in a manner similar to Example 1, Step 3 to yield the title product 8-(1-hydroxyethyl)-3,6-dimethyl-2-(2-methylpropoxy)quinazolin-4-one as a solid (120 mg, 99% yield). MS: (ES⁺) m/z=291.1 [M+H]⁺.

### Step 4: Preparation of methyl 2-((1-(2-isobutoxy-3,6-dimethyl-4-oxo-3,4-dihydroquinazolin-8-yl)ethyl)amino)benzoate

A mixture of 8-(1-hydroxyethyl)-3,6-dimethyl-2-(2-methylpropoxy)quinazolin-4-one (110 mg, 0.38 mmol) and Et₃N (230 mg, 2.27 mmol) in DCM (6 mL) was cooled to 0° C., then Ms₂O (427 mg, 1.52 mmol) was added and stirred while warming to room temperature for 1 h. Methyl anthranilate (54 mg, 0.36 mmol) was added and the reaction was warmed to 50° C. and stirred overnight. The reaction was quenched by the addition of water (20 mL) and extracted with DCM (3×30 mL). The combined organic layers were washed with brine (50 mL), dried over anhydrous Na₂SO₄, filtered, concentrated under reduced pressure, and purified by silica gel chromatography, eluted with DCM:MeOH=10:1, to give the title product methyl 2-((1-(2-isobutoxy-3,6-dimethyl-4-oxo-3,4-dihydroquinazolin-8-yl)ethyl)amino)benzoate as a solid (128 mg, 80% yield). MS: (ES⁺) m/z=424.2 [M+H]⁺.

### Step 5: Preparation of Example 11: 2-((1-(2-isobutoxy-3,6-dimethyl-4-oxo-3,4-dihydroquinazolin-8-yl)ethyl)amino)benzoic acid, Enantiomer 1; and Example 12: 2-((1-(2-isobutoxy-3,6-dimethyl-4-oxo-3,4-dihydroquinazolin-8-yl)ethyl)amino)benzoic acid, Enantiomer 2

The racemic product 2-((1-(2-isobutoxy-3,6-dimethyl-4-oxo-3,4-dihydroquinazolin-8-yl)ethyl)amino)benzoic acid was prepared in a manner similar to Example 1, Step 5, then separated via chiral preparative HPLC (column: CHIRALPAK IF, 2×25 cm, 5 μm; mobile phase: hexanes (0.1% FA):IPA=98:2; flow rate: 20 mL/min) to yield Example 11: 2-((1-(2-isobutoxy-3,6-dimethyl-4-oxo-3,4-dihydroquinazolin-8-yl)ethyl)amino)benzoic acid, Enantiomer 1 (3.5 mg, 19% yield, >99% ee), ¹H NMR (methanol-d₄, 300 MHz): δ 7.88 (dd, J=7.9 Hz, 1H), 7.79 (s, 1H), 7.52 (s, 1H), 7.10 (t, J=7.7 Hz, 1H), 6.48 (t, J=7.5 Hz, 1H), 6.43 (d, J=8.4 Hz, 1H), 5.45 (q, J=13.5 Hz, 1H), 4.37 (dd, J=6.6 Hz, 2H), 3.54 (s, 3H), 2.34 (s, 3H), 2.30-2.15 (m, 1H), 1.60 (d, J=6.6 Hz, 3H), 1.08 (d, J=1.2 Hz, 6H), MS (ES⁺) m/z=410.2 [M+H]⁺; and Example 12: 2-((1-(2-isobutoxy-3,6-dimethyl-4-oxo-3,4-dihydroquinazolin-8-yl)ethyl)amino)benzoic acid, Enantiomer 2 (4.5 mg, 25% yield, 95% ee), ¹H NMR (methanol-d₄, 300 MHz): δ 7.88 (dd, J=7.9 Hz, 1H), 7.79 (s, 1H), 7.52 (s, 1H), 7.10 (t, J=7.7 Hz, 1H), 6.48 (t, J=7.5 Hz, 1H), 6.43 (d, J=8.4 Hz, 1H), 5.45 (q, J=13.5 Hz, 1H), 4.37 (dd, J=6.6 Hz, 2H), 3.54 (s, 3H), 2.34 (s, 3H), 2.30-2.15 (m, 1H), 1.60 (d, J=6.6 Hz, 3H), 1.08 (d, J=1.2 Hz, 6H), MS (ES⁺) m/z=410.2 [M+H]⁺.

### Example 13: 5-chloro-2-((1-(2-(5-fluoroisoindolin-2-yl)-3,6-dimethyl-4-oxo-3,4-dihydroquinazolin-8-yl)ethyl)amino)benzoic acid, Enantiomer 1; and

### Example 14: 5-chloro-2-((1-(2-(5-fluoroisoindolin-2-yl)-3,6-dimethyl-4-oxo-3,4-dihydroquinazolin-8-yl)ethyl)amino)benzoic acid, Enantiomer 2

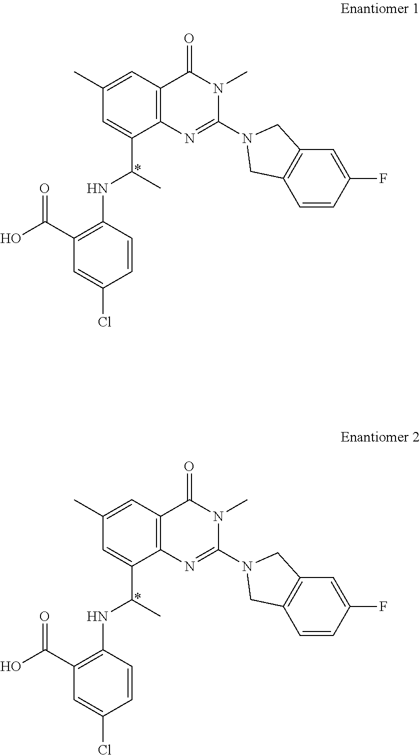 [SMILES: Cc1cc(C(C)Nc2ccc(Cl)cc2C(=O)O)c2nc(N3Cc4ccc(F)cc4C3)n(C)c(=O)c2c1 ; Cc1cc(C(C)Nc2ccc(Cl)cc2C(=O)O)c2nc(N3Cc4ccc(F)cc4C3)n(C)c(=O)c2c1]

### Step 1: Preparation of 8-bromo-2-(5-fluoro-1,3-dihydroisoindol-2-yl)-3,6-dimethylquinazolin-4-one

Performed in a manner similar to Example 1, Step 1 with 5-fluoroisoindoline hydrochloride as the amine hydrochloride salt to afford the title compound 8-bromo-2-(5-fluoro-1,3-dihydroisoindol-2-yl)-3,6-dimethylquinazolin-4-one (800 mg, 90% yield) as a solid. MS: (ES⁺) m/z=388.0 [M+H]⁺.

### Step 2: Preparation of 8-acetyl-2-(5-fluoro-1,3-dihydroisoindol-2-yl)-3,6-dimethylquinazolin-4-one

Performed in a manner similar to Example 1, Step 2 to afford the title compound 8-acetyl-2-(5-fluoro-1,3-dihydroisoindol-2-yl)-3,6-dimethylquinazolin-4-one (300 mg, 40% yield) as a solid. MS: (ES⁺) m/z=352.1 [M+H]⁺.

### Step 3: Preparation of 2-(5-fluoro-1,3-dihydroisoindol-2-yl)-8-(1-hydroxyethyl)-3,6-dimethylquinazolin-4-one

Performed in a manner similar to Example 1, Step 3 to yield 2-(5-fluoro-1,3-dihydroisoindol-2-yl)-8-(1-hydroxyethyl)-3,6-dimethylquinazolin-4-one (284 mg, 91% yield) as an oil. MS: (ES⁺) m/z=354.2 [M+H]⁺.

### Step 4: Preparation of methyl 5-chloro-2-((1-(2-(5-fluoroisoindolin-2-yl)-3,6-dimethyl-4-oxo-3,4-dihydroquinazolin-8-yl)ethyl)amino)benzoate

Performed in a manner similar to Example 7, Step 4 to yield methyl 5-chloro-2-((1-(2-(5-fluoroisoindolin-2-yl)-3,6-dimethyl-4-oxo-3,4-dihydroquinazolin-8-yl)ethyl)amino)benzoate (334 mg, 65% yield) as an oil. MS: (ES⁺) m/z=521.1 [M+H]⁺.

### Step 5: Preparation of Example 13: 5-chloro-2-((1-(2-(5-fluoroisoindolin-2-yl)-3,6-dimethyl-4-oxo-3,4-dihydroquinazolin-8-yl)ethyl)amino)benzoic acid, Enantiomer 1; and Example 14: 5-chloro-2-((1-(2-(5-fluoroisoindolin-2-yl)-3,6-dimethyl-4-oxo-3,4-dihydroquinazolin-8-yl)ethyl)amino)benzoic acid, Enantiomer 2

The racemic product 5-chloro-2-((1-(2-(5-fluoroisoindolin-2-yl)-3,6-dimethyl-4-oxo-3,4-dihydroquinazolin-8-yl)ethyl)amino)benzoic acid was prepared in a manner similar to Example 1, Step 5, then separated by chiral preparative HPLC (column: Lux 5 μm Cellulose-4, 2.12×25 cm, 5 μm; mobile phase: hexanes (0.1% FA):EtOH=80:20; flow rate: 20 mL/min) to afford the title compounds Example 13: 5-chloro-2-((1-(2-(5-fluoroisoindolin-2-yl)-3,6-dimethyl-4-oxo-3,4-dihydroquinazolin-8-yl)ethyl)amino)benzoic acid, Enantiomer 1 (32.6 mg, 10% yield, >99% ee) as a solid, ¹H NMR (methanol-d₄, 300 MHz): δ 7.84-7.65 (m, 2H), 7.44 (d, J=2.1 Hz, 1H), 7.31 (dd, J=8.3, 4.9 Hz, 1H), 7.13-6.82 (m, 3H), 6.40 (d, J=9.1 Hz, 1H), 5.43 (q, J=6.6 Hz, 1H), 5.07-4.87 (m, 4H), 3.65 (s, 3H), 2.31 (s, 3H), 1.58 (d, J=6.6 Hz, 3H), MS: (ES⁺) m/z=507.2 [M+H]⁺; and Example 14: 5-chloro-2-((1-(2-(5-fluoroisoindolin-2-yl)-3,6-dimethyl-4-oxo-3,4-dihydroquinazolin-8-yl)ethyl)amino)benzoic acid, Enantiomer 2 (51 mg, 15% yield, 99% ee), ¹H NMR (methanol-d₄, 300 MHz): δ 7.84-7.65 (m, 2H), 7.44 (d, J=2.1 Hz, 1H), 7.31 (dd, J=8.4, 4.9 Hz, 1H), 7.13-6.92 (m, 3H), 6.40 (d, J=9.1 Hz, 1H), 5.43 (q, J=6.6 Hz, 1H), 5.14-4.89 (m, 4H), 3.65 (s, 3H), 2.31 (s, 3H), 1.58 (d, J=6.7 Hz, 3H), MS: (ES⁺) m/z=507.2 [M+H]⁺.

### Example 15: 5-cyano-2-((1-(2-(4,4-dimethylpiperidin-1-yl)-3,6-dimethyl-4-oxo-3,4-dihydroquinazolin-8-yl)ethyl)amino)benzoic acid, Enantiomer 1; and

### Example 16: 5-cyano-2-((1-(2-(4,4-dimethylpiperidin-1-yl)-3,6-dimethyl-4-oxo-3,4-dihydroquinazolin-8-yl)ethyl)amino)benzoic acid, Enantiomer 2

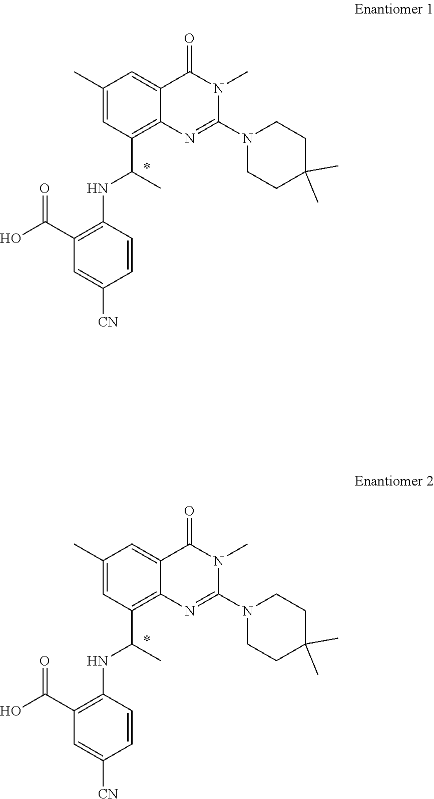 [SMILES: Cc1cc(C(C)Nc2ccc(C#N)cc2C(=O)O)c2nc(N3CCC(C)(C)CC3)n(C)c(=O)c2c1 ; Cc1cc(C(C)Nc2ccc(C#N)cc2C(=O)O)c2nc(N3CCC(C)(C)CC3)n(C)c(=O)c2c1]

### Step 1: Preparation of methyl 5-cyano-2-((1-(2-(4,4-dimethylpiperidin-1-yl)-3,6-dimethyl-4-oxo-3,4-dihydroquinazolin-8-yl)ethyl)amino)benzoate

Prepared in a manner similar to Example 7, Step 4 from 2-(4,4-dimethylpiperidin-1-yl)-8-(1-hydroxyethyl)-3,6-dimethylquinazolin-4-one (prepared in Example 19, Step 3) and methyl 2-amino-5-cyanobenzoate to yield methyl 5-cyano-2-((1-(2-(4,4-dimethylpiperidin-1-yl)-3,6-dimethyl-4-oxo-3,4-dihydroquinazolin-8-yl)ethyl)amino)benzoate (130 mg, 29% yield) as a solid. MS: (ES⁺) m/z=488.3 [M+H]⁺.

### Step 2: Preparation of 5-cyano-2-((1-(2-(4,4-dimethylpiperidin-1-yl)-3,6-dimethyl-4-oxo-3,4-dihydroquinazolin-8-yl)ethyl)amino)benzoic acid, Enantiomers 1 and 2

To a stirred solution of methyl 5-cyano-2-((1-(2-(4,4-dimethylpiperidin-1-yl)-3,6-dimethyl-4-oxo-3,4-dihydroquinazolin-8-yl)ethyl)amino)benzoate (130 mg, 0.27 mmol) in THE (5 mL) and water (5 mL) was added LiOH hydrate (447 mg, 10.7 mmol). The reaction was warmed to 50° C. and stirred for 12 h, then diluted with water (20 mL) and extracted with EtOAc (3×30 mL). The combined organic layers were washed with brine (30 mL), dried over anhydrous Na₂SO₄, filtered, and concentrated under reduced pressure. The residue was purified by reverse-phase flash chromatography (column: C18 silica gel; mobile phase: 10-100% ACN in water over 30 min) to isolate racemic product 5-cyano-2-((1-(2-(4,4-dimethylpiperidin-1-yl)-3,6-dimethyl-4-oxo-3,4-dihydroquinazolin-8-yl)ethyl)amino)benzoic acid (90 mg, 71% yield) as a solid, which was then separated via chiral preparative HPLC (column: CHIRALPAK IE, 2×25 cm, 5 μm; mobile phase: hexane (0.1% FA):EtOH=80:20; flow rate: 20 mL/min) to afford Example 15: 5-cyano-2-((1-(2-(4,4-dimethylpiperidin-1-yl)-3,6-dimethyl-4-oxo-3,4-dihydroquinazolin-8-yl)ethyl)amino)benzoic acid, Enantiomer 1 (36.4 mg, 29% yield, >99% ee) as a solid, ¹H NMR (methanol-d₄, 300 MHz): δ 8.18 (d, J=2.1 Hz, 1H), 7.81 (dd, J=2.0, 0.9 Hz, 1H), 7.49 (d, J=2.1 Hz, 1H), 7.40 (dd, J=8.9, 2.1 Hz, 1H), 6.63 (d, J=9.0 Hz, 1H), 5.53 (q, J=6.7 Hz, 1H), 3.58 (s, 3H), 3.32-3.23 (m, 4H), 2.37 (s, 3H), 1.66 (d, J=6.7 Hz, 3H), 1.63-1.53 (m, 4H), 1.06 (s, 6H), MS: (ES⁺) m/z=474.2 [M+H]⁺; and Example 16: 5-cyano-2-((1-(2-(4,4-dimethylpiperidin-1-yl)-3,6-dimethyl-4-oxo-3,4-dihydroquinazolin-8-yl)ethyl)amino)benzoic acid, Enantiomer 2 (34.3 mg, 26% yield, 99% ee), ¹H NMR (methanol-d₄, 300 MHz): δ 8.18 (d, J=2.1 Hz, 1H), 7.81 (dd, J=2.1, 0.9 Hz, 1H), 7.49 (d, J=2.1 Hz, 1H), 7.40 (dd, J=8.9, 2.1 Hz, 1H), 6.62 (d, J=9.0 Hz, 1H), 5.53 (q, J=6.7 Hz, 1H), 3.58 (s, 3H), 3.33-3.23 (m, 4H), 2.37 (s, 3H), 1.66 (d, J=6.7 Hz, 3H), 1.63-1.54 (m, 4H), 1.06 (s, 6H) MS: (ES⁺) m/z=474.2 [M+H]⁺.

### Example 17: 2-((1-(2-(5-cyanoisoindolin-2-yl)-3,6-dimethyl-4-oxo-3,4-dihydroquinazolin-8-yl)ethyl)amino)benzoic acid, Enantiomer 1; and

### Example 18: 2-((1-(2-(5-cyanoisoindolin-2-yl)-3,6-dimethyl-4-oxo-3,4-dihydroquinazolin-8-yl)ethyl)amino)benzoic acid, Enantiomer 2

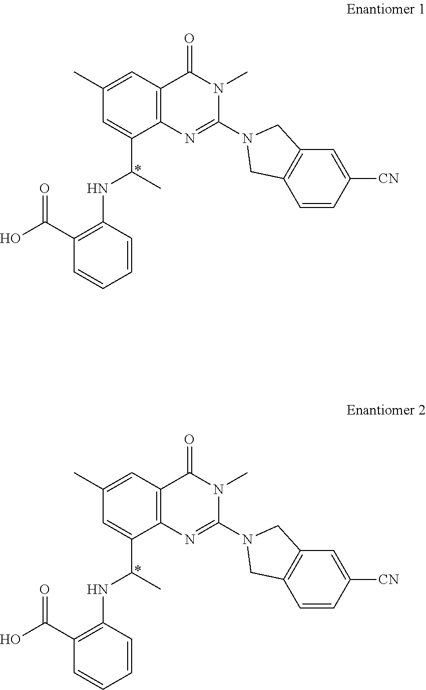 [SMILES: Cc1cc(C(C)Nc2ccccc2C(=O)O)c2nc(N3Cc4ccc(C#N)cc4C3)n(C)c(=O)c2c1 ; Cc1cc(C(C)Nc2ccccc2C(=O)O)c2nc(N3Cc4ccc(C#N)cc4C3)n(C)c(=O)c2c1]

### Step 1: Preparation of methyl 2-((1-(2-(5-cyanoisoindolin-2-yl)-3,6-dimethyl-4-oxo-3,4-dihydroquinazolin-8-yl)ethyl)amino)benzoate

To a stirred solution of methyl 2-((1-(2-(5-chloroisoindolin-2-yl)-3,6-dimethyl-4-oxo-3,4-dihydroquinazolin-8-yl)ethyl)amino)benzoate (550 mg, 1.09 mmol) and tBuXphos Pd G3 (174 mg, 0.22 mmol) in H₂O (2 mL) and DMF (4 mL) were added t-BuXphos (93 mg, 0.22 mmol) and Zn(CN)₂ (154 mg, 1.3 mmol). The resulting mixture was stirred for 12 h at 70° C. under a N₂ atmosphere. The resulting mixture was diluted with H₂O (50 mL). The resulting mixture was extracted with EtOAc (3×50 mL). The combined organic layers were washed with brine (1×50 mL) and then dried over anhydrous Na₂SO₄. After filtration, the filtrate was concentrated under reduced pressure. The residue was purified by Prep-TLC (petroleum ether/ethyl acetate=1:1) to afford methyl 2-((1-(2-(5-cyanoisoindolin-2-yl)-3,6-dimethyl-4-oxo-3,4-dihydroquinazolin-8-yl)ethyl)amino)benzoate (350 mg, 65% yield) as a solid. MS: (ES⁺) m/z=494.3 [M+H]⁺.

### Step 2: Preparation of 2-((1-(2-(5-cyanoisoindolin-2-yl)-3,6-dimethyl-4-oxo-3,4-dihydroquinazolin-8-yl)ethyl)amino)benzoic acid, Enantiomers 1 and 2

The title compound was prepared using methods similar to those described in Example 7, Step 5 to afford Example 17: 2-((1-(2-(5-cyanoisoindolin-2-yl)-3,6-dimethyl-4-oxo-3,4-dihydroquinazolin-8-yl)ethyl)amino)benzoic acid, Enantiomer 1 (6.4 mg, 3% yield, 99% ee), ¹H NMR (methanol-d₄, 300 MHz): δ 7.91-7.82 (m, 1H), 7.82-7.72 (m, 2H), 7.72-7.64 (m, 1H), 7.60-7.45 (m, 2H), 7.14-7.02 (m, 1H), 6.53-6.39 (m, 2H), 5.55-5.42 (m, 1H), 5.22-5.00 (m, 4H), 3.71 (s, 3H), 2.34 (s, 3H), 1.62 (d, J=6.7 Hz, 3H), MS: (ES⁺) m/z=480.1 [M+H]⁺; and Example 18: 2-((1-(2-(5-cyanoisoindolin-2-yl)-3,6-dimethyl-4-oxo-3,4-dihydroquinazolin-8-yl)ethyl)amino)benzoic acid, Enantiomer 2 (6.3 mg, 3% yield, >95% ee), ¹H NMR (methanol-d₄, 300 MHz): δ 7.91-7.82 (m, 1H), 7.82-7.72 (m, 2H), 7.72-7.64 (m, 1H), 7.60-7.45 (m, 2H), 7.14-7.04 (m, 1H), 6.54-6.40 (m, 2H), 5.55-5.42 (m, 1H), 5.22-5.01 (m, 4H), 3.71 (s, 3H), 2.35 (s, 3H), 1.62 (d, J=6.7 Hz, 3H), MS: (ES⁺) m/z=480.1 [M+H]⁺.

### Example 19: 2-((1-(2-(5-carbamoylisoindolin-2-yl)-3,6-dimethyl-4-oxo-3,4-dihydroquinazolin-8-yl)ethyl)amino)benzoic acid

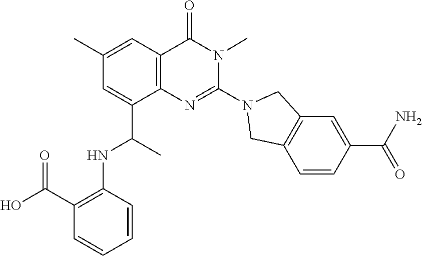 [SMILES: Cc1cc(C(C)Nc2ccccc2C(=O)O)c2nc(N3Cc4ccc(C(N)=O)cc4C3)n(C)c(=O)c2c1]

### Step 1: Preparation 2-((1-(2-(5-carbamoylisoindolin-2-yl)-3,6-dimethyl-4-oxo-3,4-dihydroquinazolin-8-yl)ethyl)amino)benzoic acid

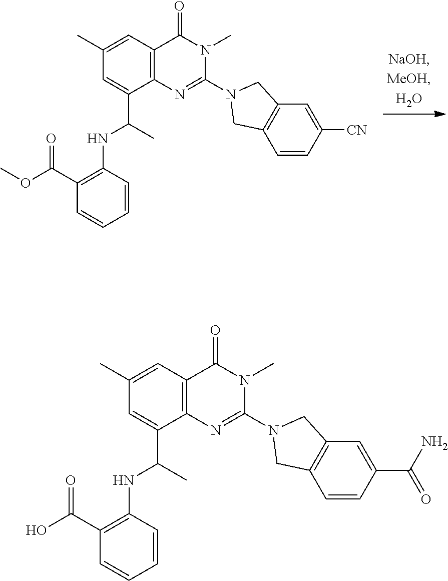 [SMILES: COC(=O)c1ccccc1NC(C)c1cc(C)cc2c(=O)n(C)c(N3Cc4ccc(C#N)cc4C3)nc12 ; Cc1cc(C(C)Nc2ccccc2C(=O)O)c2nc(N3Cc4ccc(C(N)=O)cc4C3)n(C)c(=O)c2c1]

To a stirred solution of methyl 2-((1-(2-(5-cyanoisoindolin-2-yl)-3,6-dimethyl-4-oxo-3,4-dihydroquinazolin-8-yl)ethyl)amino)benzoate (from Example 18, Step 1) (100 mg, 0.2 mmol) in MeOH (1 mL) and H₂O (0.5 mL) was added NaOH (162 mg, 4.1 mmol). The resulting mixture was stirred for 6 h at 50° C. The resulting mixture was diluted with H₂O (30 mL). The resulting mixture was extracted with DCM (3×30 mL). The combined organic layers were washed with brine (1×30 mL) and then dried over anhydrous Na₂SO₄. After filtration, the filtrate was concentrated under reduced pressure. The residue was purified by Prep-TLC (eluted with PE:EA=1:1) and Prep-HPLC with the following conditions (Column: XBridge Prep OBD C18 Column, 30*150 mm, 5 μm; Mobile Phase A: 10 mmol NH₄HCO₃+0.05% NH₄OH, Mobile Phase B: ACN; Gradient: 15% B to 40% B in 9 min, Flow rate: 60 mL/min) to afford Example 19: 2-((1-(2-(5-carbamoylisoindolin-2-yl)-3,6-dimethyl-4-oxo-3,4-dihydroquinazolin-8-yl)ethyl)amino)benzoic acid (10.2 mg, 10% yield). ¹H NMR (methanol-d₄, 300 MHz): δ 8.70 (s, 1H), 7.97 (s, 1H), 7.88 (d, J=1.6 Hz, 1H), 7.86-7.73 (m, 2H), 7.68 (d, J=2.0 Hz, 1H), 7.49-7.39 (m, 2H), 7.36 (s, 1H), 7.13-7.02 (m, 1H), 6.49-6.34 (m, 2H), 5.40 (d, J=7.0 Hz, 1H), 5.18-4.99 (m, 4H), 3.61 (s, 3H), 2.29 (s, 3H), 1.55 (d, J=6.5 Hz, 3H), MS: (ES⁺) m/z=498.2 [M+H]⁻.

### Example 20: 2-((1-(7-fluoro-3,6-dimethyl-4-oxo-2-(4-(2,2,2-trifluoroethyl)piperazin-1-yl)-3,4-dihydroquinazolin-8-yl)ethyl)amino)benzoic acid (Enantiomer 1); and

### Example 21: 2-((1-(7-fluoro-3,6-dimethyl-4-oxo-2-(4-(2,2,2-trifluoroethyl)piperazin-1-yl)-3,4-dihydroquinazolin-8-yl)ethyl)amino)benzoic acid (Enantiomer 2)

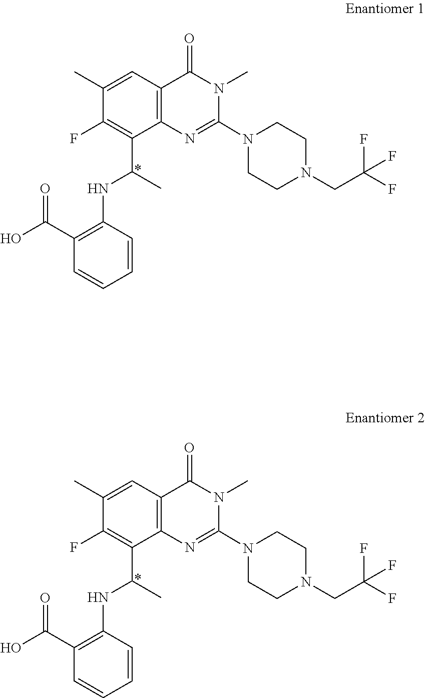 [SMILES: Cc1cc2c(=O)n(C)c(N3CCN(CC(F)(F)F)CC3)nc2c(C(C)Nc2ccccc2C(=O)O)c1F ; Cc1cc2c(=O)n(C)c(N3CCN(CC(F)(F)F)CC3)nc2c(C(C)Nc2ccccc2C(=O)O)c1F]

### Step 1: Preparation of 6-amino-3-bromo-2-fluoro-N-methylbenzamide

Into a 1 L round-bottom flask were added 6-amino-3-bromo-2-fluorobenzoic acid (20 g, 21.4 mmol), methylamine hydrochloride (8.66 g, 32 mmol), DCM (200 mL), DIEA (44.6 mL, 64.1 mmol) and HATU (48.8 g, 32 mmol) at room temperature. The mixture was stirred at room temperature for 1 h. The resulting mixture was extracted with CH₂Cl₂ (3×200 mL). The combined organic layers were washed with brine (3×200 mL) and dried over anhydrous Na₂SO₄. After filtration, the filtrate was concentrated under reduced pressure. The residue was purified by silica gel column chromatography (eluted with PE:EA=1:1) to afford 6-amino-3-bromo-2-fluoro-N-methylbenzamide (19.3 g, 92% yield) as a light-yellow oil. MS: (ES⁺) m/z=249.0 [M+H]⁺.

### Step 2: Preparation of 6-amino-2-fluoro-N,3-dimethylbenzamide

Into a 1000 mL round-bottom flask were added 6-amino-3-bromo-2-fluoro-N-methylbenzamide (19.3 g, 19.5 mmol), dioxane (360 mL), H₂O (40 mL), methylboronic acid (8 g, 33.4 mmol), K₃PO₄ (49.8 g, 58.7 mmol) and Pd(dppf)Cl₂ (2.86 g, 0.98 mmol). The mixture was stirred at 110° C. for 2 h under a N₂ atmosphere. The reaction was quenched with water (400 mL) at room temperature. The resulting mixture was extracted with CH₂Cl₂ (3×200 mL). The combined organic layers were washed with brine (3×200 mL) and dried over anhydrous Na₂SO₄. After filtration, the filtrate was concentrated under reduced pressure. The residue was purified by silica gel column chromatography (eluted with PE:EA=1:1) to afford 6-amino-2-fluoro-N,3-dimethylbenzamide) (8.40 g, 59% yield) as a white solid. MS: (ES⁺) m/z=183.1 [M+H]⁺.

### Step 3: Preparation of 2-amino-3-bromo-4-fluoro-N,5-dimethylbenzamide

Into a 500 mL round-bottom flask were added 2-amino-4-fluoro-N,5-dimethylbenzamide (8.4 g, 46.1 mmol), MeCN (100 mL) and NBS (8.21 g, 46.1 mmol). The mixture was stirred at 75° C. for 4 h. The reaction was quenched with water (50 mL) at room temperature. The resulting mixture was extracted with CH₂Cl₂ (3×200 mL). The combined organic layers were washed with brine (3×100 mL) and dried over anhydrous Na₂SO₄. After filtration, the filtrate was concentrated under reduced pressure. The residue was purified by silica gel column chromatography (eluted with PE:EA=1:1) to afford 2-amino-3-bromo-4-fluoro-N,5-dimethylbenzamide (14.6 g, >95% crude yield) as a light-yellow solid. MS: (ES⁺) m/z=263.0 [M+H]⁺.

### Step 4: Preparation of 2-((1-(7-fluoro-3,6-dimethyl-4-oxo-2-(4-(2,2,2-trifluoroethyl)piperazin-1-yl)-3,4-dihydroquinazolin-8-yl)ethyl)amino)benzoic acid (Enantiomers 1 and 2)

The title compounds were prepared from 2-amino-3-bromo-4-fluoro-N,5-dimethylbenzamide using methods similar to those described in Examples 7 and 8, utilizing 8-bromo-2-chloro-7-fluoro-3,6-dimethylquinazolin-4(3H)-one, which was furnished from 2-amino-3-bromo-4-fluoro-N,5-dimethylbenzamide via methods similar to those described in Intermediate 1 (Steps 2 and 3), to afford Example 20: 2-((1-(7-fluoro-3,6-dimethyl-4-oxo-2-(4-(2,2,2-trifluoroethyl)piperazin-1-yl)-3,4-dihydroquinazolin-8-yl)ethyl)amino)benzoic acid (Enantiomer 1) (15.6 mg, 7% yield, >99% ee), ¹H NMR (400 MHz, methanol-d₄) δ 7.89-7.82 (m, 2H), 7.21-7.17 (m, 1H), 6.83 (d, J=8.5 Hz, 1H), 6.52-6.44 (m, 1H), 5.68 (d, J=7.3 Hz, 1H), 3.58 (s, 3H), 3.52-3.43 (m, 4H), 3.25-3.15 (m, 2H), 3.00-2.94 (m, 4H), 2.33 (d, J=2.4 Hz, 3H), 1.76 (d, J=6.9 Hz, 3H), MS: (ES⁺) m/z=522.4 [M+H]⁺; and Example 21: 2-((1-(7-fluoro-3,6-dimethyl-4-oxo-2-(4-(2,2,2-trifluoroethyl)piperazin-1-yl)-3,4-dihydroquinazolin-8-yl)ethyl)amino)benzoic acid (Enantiomer 2) (18.9 mg, 9% yield, 98.4% ee), ¹H NMR (400 MHz, methanol-d₄) δ 7.89-7.82 (m, 2H), 7.21-7.17 (m, 1H), 6.83 d, J=8.5 Hz, 1H), 6.52-6.44 (m, 1H), 5.68 (m, 1H), 3.58 (s, 3H), 3.52-3.44 (m, 4H), 3.25-3.15 (m, 2H), 3.00-2.94 (m, 4H), 2.33 (d, J=2.1 Hz, 3H), 1.76 (d, J=6.9 Hz, 3H), MS: (ES⁺) m/z=522.4 [M+H]⁺.

### Example 22: 2-((1-(6-chloro-2-(4,4-dimethylpiperidin-1-yl)-3-methyl-4-oxo-3,4-dihydroquinazolin-8-yl)ethyl)amino)benzoic acid (Enantiomer 1); and

### Example 23: 2-((1-(6-chloro-2-(4,4-dimethylpiperidin-1-yl)-3-methyl-4-oxo-3,4-dihydroquinazolin-8-yl)ethyl)amino)benzoic acid (Enantiomer 2)

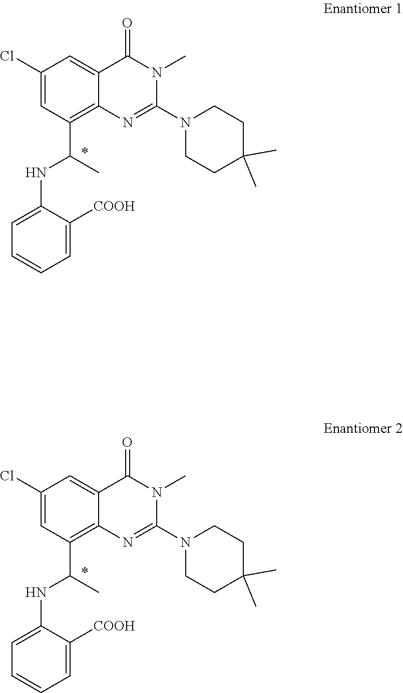 [SMILES: CC(Nc1ccccc1C(=O)O)c1cc(Cl)cc2c(=O)n(C)c(N3CCC(C)(C)CC3)nc12 ; CC(Nc1ccccc1C(=O)O)c1cc(Cl)cc2c(=O)n(C)c(N3CCC(C)(C)CC3)nc12]

The title compounds were prepared using methods similar to those described in Examples 7 and 8, using Intermediate 5, to afford Example 22: 2-((1-(6-chloro-2-(4,4-dimethylpiperidin-1-yl)-3-methyl-4-oxo-3,4-dihydroquinazolin-8-yl)ethyl)amino)benzoic acid (Enantiomer 1) (3.2 mg, 21% yield, 99% ee) as a white solid. ¹H NMR (400 MHz, DMSO-d₆) δ 8.70-8.65 (m, 1H), 7.85-7.74 (m, 2H), 7.60 (s, 1H), 7.22-7.18 (m, 1H), 6.55-6.50 (m, 1H), 6.50-6.45 (m, 1H), 5.45-5.35 (m, 1H), 3.50 (s, 3H), 3.22-3.18 (m, 4H), 2.55-3.45 (m, 7H), 0.95 (s, 6H), MS: (ES⁺) m/z=469.1 [M+H]⁺; and Example 23: 2-((1-(6-chloro-2-(4,4-dimethylpiperidin-1-yl)-3-methyl-4-oxo-3,4-dihydroquinazolin-8-yl)ethyl)amino)benzoic acid (Enantiomer 2) (3.8 mg, 25% yield, 99% ee) as a white solid. ¹H NMR (400 MHz, DMSO-d₆) δ 8.70-8.65 (m, 1H), 7.85-7.78 (m, 2H), 7.60 (s, 1H), 7.22-7.18 (m, 1H), 6.55-6.50 (m, 1H), 6.50-6.45 (m, 1H), 5.45-5.35 (m, 1H), 3.50 (s, 3H), 3.22-3.18 (m, 4H), 2.55-3.45 (m, 7H), 0.95 (s, 6H), MS: (ES⁺) m/z=469.1 [M+H]⁺.

### Example 24: 2-((1-(6-chloro-3-methyl-4-oxo-2-(4-(2,2,2-trifluoroethyl)piperazin-1-yl)-3,4-dihydroquinazolin-8-yl)ethyl)amino)-5-fluorobenzoic acid (Enantiomer 1); and

### Example 25: 2-((1-(6-chloro-3-methyl-4-oxo-2-(4-(2,2,2-trifluoroethyl)piperazin-1-yl)-3,4-dihydroquinazolin-8-yl)ethyl)amino)-5-fluorobenzoic acid (Enantiomer 2)

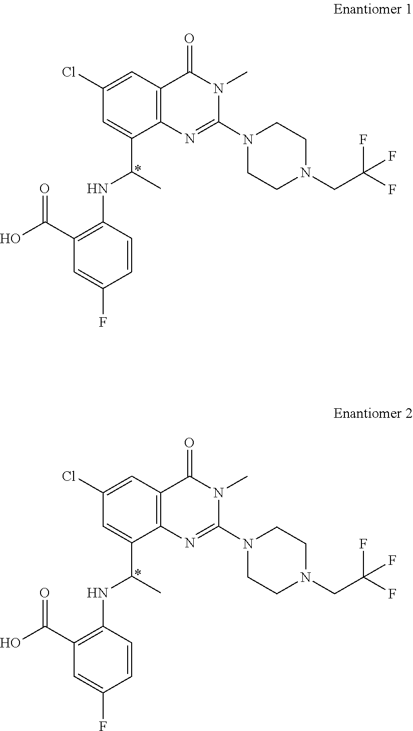 [SMILES: CC(Nc1ccc(F)cc1C(=O)O)c1cc(Cl)cc2c(=O)n(C)c(N3CCN(CC(F)(F)F)CC3)nc12 ; CC(Nc1ccc(F)cc1C(=O)O)c1cc(Cl)cc2c(=O)n(C)c(N3CCN(CC(F)(F)F)CC3)nc12]

The title compounds were prepared using methods similar to those described in Examples 7 and 8, using Intermediate 5, to afford Example 24: 2-((1-(6-chloro-3-methyl-4-oxo-2-(4-(2,2,2-trifluoroethyl)piperazin-1-yl)-3,4-dihydroquinazolin-8-yl)ethyl)amino)-5-fluorobenzoic acid, Enantiomer 1 (10.4 mg, 18% yield, 99% ee). ¹H NMR (DMSO-d₆, 400 MHz): δ 13.10 (s, 1H), 8.26 (s, 1H), 7.84 (s, 1H), 7.59 (s 1H), 7.55-7.50 (m, 1H), 7.18-7.10 (m, 1H), 6.44-4.40 (m, 1H), 5.40-5.30 (m, 1H), 3.48 (s, 3H), 3.40-3.20 (m, 6H), 2.83-2.60 (m, 4H), 1.59 (d, J=6.6 Hz, 3H), MS: (ES⁺) m/z=542.1 [M+H]⁺; and Example 25: 2-((1-(6-chloro-3-methyl-4-oxo-2-(4-(2,2,2-trifluoroethyl)piperazin-1-yl)-3,4-dihydroquinazolin-8-yl)ethyl)amino)-5-fluorobenzoic acid, Enantiomer 2 (10.9 mg, 19% yield, 99% ee) as a white solid. ¹H NMR (DMSO-d₆, 400 MHz): δ 13.10 (s, 1H), 8.26 (s, 1H), 7.84 (s, 1H), 7.59 (s 1H), 7.55-7.50 (m, 1H), 7.18-7.10 (m, 1H), 6.44-4.40 (m, 1H), 5.40-5.30 (m, 1H), 3.48 (s, 3H), 3.40-3.20 (m, 6H), 2.83-2.60 (m, 4H), 1.59 (d, J=6.6 Hz, 3H), MS: (ES⁺) m/z=542.1 [M+H]⁺.

### Example 26: 2-((1-(6-chloro-3-methyl-4-oxo-2-(4-(2,2,2-trifluoroethyl)piperazin-1-yl)-3,4-dihydroquinazolin-8-yl)ethyl)amino)benzoic acid (Enantiomer 1); and

### Example 27: 2-((1-(6-chloro-3-methyl-4-oxo-2-(4-(2,2,2-trifluoroethyl)piperazin-1-yl)-3,4-dihydroquinazolin-8-yl)ethyl)amino)benzoic acid (Enantiomer 2)

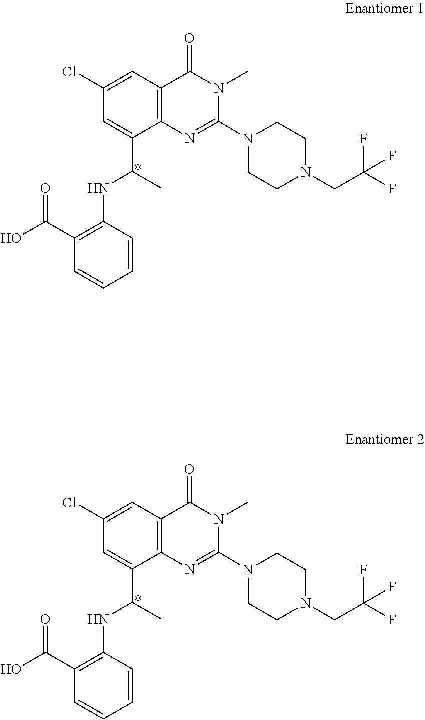 [SMILES: CC(Nc1ccccc1C(=O)O)c1cc(Cl)cc2c(=O)n(C)c(N3CCN(CC(F)(F)F)CC3)nc12 ; CC(Nc1ccccc1C(=O)O)c1cc(Cl)cc2c(=O)n(C)c(N3CCN(CC(F)(F)F)CC3)nc12]

The title compounds were prepared using methods similar to those described in Examples 7 and 8, using Intermediate 5, to afford Example 26: 2-((1-(6-chloro-3-methyl-4-oxo-2-(4-(2,2,2-trifluoroethyl)piperazin-1-yl)-3,4-dihydroquinazolin-8-yl)ethyl)amino)benzoic acid (Enantiomer 1) (31.4 mg, 18% yield, 99% ee) as a white solid. ¹H NMR (400 MHz, DMSO-d₆) δ 12.70 (s, 1H), 8.40 (s, 1H), 7.84 (s, 1H), 7.79 (d, J=7.8 Hz, 1H), 7.58 (s, 1H), 7.25-7.20 (m, 1H), 6.52-6.48 (m, 1H), 6.44-6.40 (m, 1H), 5.38-5.25 (m, 1H), 3.48 (s, 3H), 3.30-3.20 (m, 6H), 2.90-2.85 (m, 4H), 1.59 (d, J=6.5 Hz, 3H), MS: (ES⁺) m/z=524.1 [M+H]⁺; and Example 27: 2-((1-(6-chloro-3-methyl-4-oxo-2-(4-(2,2,2-trifluoroethyl)piperazin-1-yl)-3,4-dihydroquinazolin-8-yl)ethyl)amino)benzoic acid, Enantiomer 2 (36.6 mg, 21% yield, 99% ee) as a white solid. ¹H NMR (400 MHz, DMSO-d₆) δ 12.70 (s, 1H), 8.40 (s, 1H), 7.84 (s, 1H), 7.79 (d, J=7.8 Hz, 1H), 7.58 (s, 1H), 7.25-7.20 (m, 1H), 6.52-6.48 (m, 1H), 6.44-6.40 (m, 1H), 5.38-5.25 (m, 1H), 3.48 (s, 3H), 3.30-3.18 (m, 6H), 2.90-2.85 (m, 4H), 1.59 (d, J=6.5 Hz, 3H), MS: (ES⁺) m/z=524.1 [M+H]⁺.

### Example 28: 2-((1-(2-(4,4-dimethylpiperidin-1-yl)-3,6-dimethyl-4-oxo-3,4-dihydroquinazolin-8-yl)ethyl)amino)-6-fluorobenzoic acid

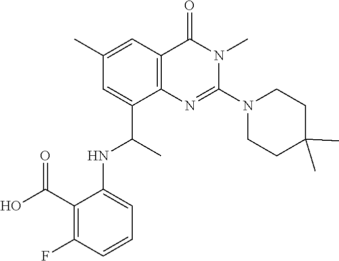 [SMILES: Cc1cc(C(C)Nc2cccc(F)c2C(=O)O)c2nc(N3CCC(C)(C)CC3)n(C)c(=O)c2c1]

### Example 29: 2-((1-(2-(4,4-dimethylpiperidin-1-yl)-3,6-dimethyl-4-oxo-3,4-dihydroquinazolin-8-yl)ethyl)amino)-5-fluorobenzoic acid (Enantiomer 1); and

### Example 30: 2-((1-(2-(4,4-dimethylpiperidin-1-yl)-3,6-dimethyl-4-oxo-3,4-dihydroquinazolin-8-yl)ethyl)amino)-5-fluorobenzoic acid (Enantiomer 2)

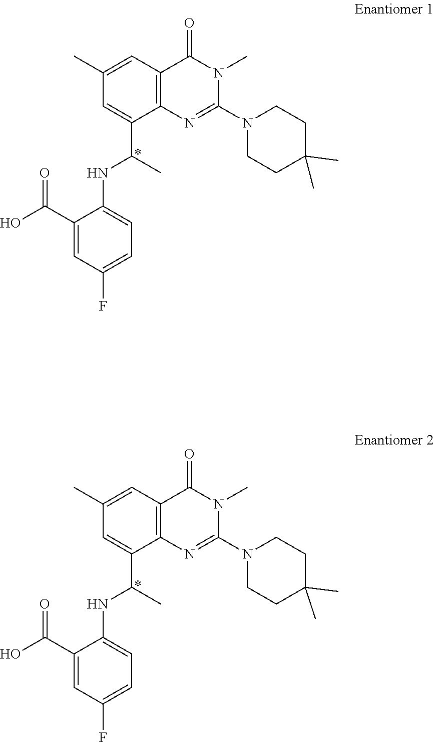 [SMILES: Cc1cc(C(C)Nc2ccc(F)cc2C(=O)O)c2nc(N3CCC(C)(C)CC3)n(C)c(=O)c2c1 ; Cc1cc(C(C)Nc2ccc(F)cc2C(=O)O)c2nc(N3CCC(C)(C)CC3)n(C)c(=O)c2c1]

The title compounds were prepared using methods similar to those described in Examples 7 and 8, to afford Example 29: 2-((1-(2-(4,4-dimethylpiperidin-1-yl)-3,6-dimethyl-4-oxo-3,4-dihydroquinazolin-8-yl)ethyl)amino)-5-fluorobenzoic acid, Enantiomer 1 (51 mg, 27% yield, 99% ee). ¹H NMR (CD₃OD-d₄, 300 MHz): 7.84 (s, 1H), 7.58-7.55 (m, 1H), 7.50 (s, 1H), 6.90-6.85 (m, 1H), 6.44-4.40 (m, 1H), 5.40-5.30 (m, 1H), 3.60 (s, 3H), 3.40-3.20 (m, 4H), 2.35 (s, 3H), 1.70-1.55 (m, 7H), 1.10 (s, 6H), MS: (ES⁺) m/z=467.1 [M+H]⁺; and Example 30: 2-((1-(2-(4,4-dimethylpiperidin-1-yl)-3,6-dimethyl-4-oxo-3,4-dihydroquinazolin-8-yl)ethyl)amino)-5-fluorobenzoic acid, Enantiomer 2 (59 mg, 31% yield, 98% ee) as a white solid. ¹H NMR (CD₃OD-d₄, 300 MHz): 7.84 (s, 1H), 7.58-7.55 (m, 1H), 7.50 (s, 1H), 6.90-6.85 (m, 1H), 6.44-4.40 (m, 1H), 5.40-5.30 (m, 1H), 3.60 (s, 3H), 3.40-3.20 (m, 4H), 2.35 (s, 3H), 1.70-1.55 (m, 7H), 1.10 (s, 6H), MS: (ES⁺) m/z=467.1 [M+H]⁺.

### Example 31: 2-((1-(2-((4-chloro-3-(trifluoromethoxy)benzyl)thio)-3,6-dimethyl-4-oxo-3,4-dihydroquinazolin-8-yl)ethyl)amino)benzoic acid, (Enantiomer 1); and

### Example 32: 2-((1-(2-((4-chloro-3-(trifluoromethoxy)benzyl)thio)-3,6-dimethyl-4-oxo-3,4-dihydroquinazolin-8-yl)ethyl)amino)benzoic acid, (Enantiomer 2)

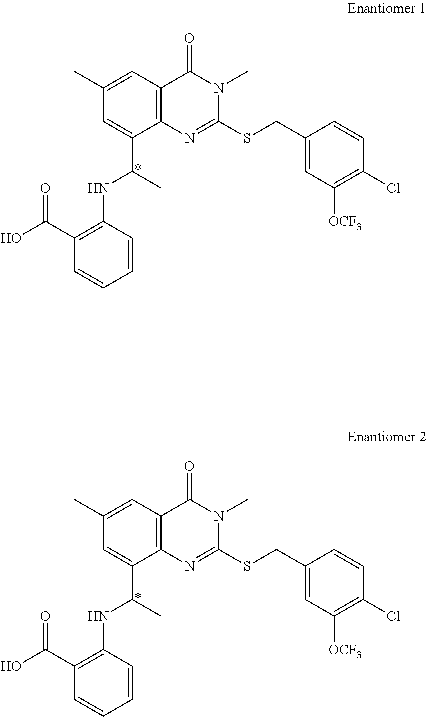 [SMILES: Cc1cc(C(C)Nc2ccccc2C(=O)O)c2nc(SCc3ccc(Cl)c(OC(F)(F)F)c3)n(C)c(=O)c2c1 ; Cc1cc(C(C)Nc2ccccc2C(=O)O)c2nc(SCc3ccc(Cl)c(OC(F)(F)F)c3)n(C)c(=O)c2c1]

### Step 1: Preparation of 8-bromo-3,6-dimethyl-2-thioxo-2,3-dihydroquinazolin-4(1H)-one

Into a 1000 mL round-bottom flask were added 2-amino-3-bromo-5-methylbenzoic acid (20 g, 86.9 mmol) and EtOH (200 mL) at room temperature. To the above mixture was added methyl isothiocyanate (12.7 g, 174 mmol), Et₃N (110 g, 1090 mmol) in portions at room temperature. The resulting mixture was stirred for an additional 4 h at 80° C. The precipitated solids were collected by filtration and washed with Et₂O (3×10 mL). The resulting solids were dried under vacuum. This resulted in 8-bromo-3,6-dimethyl-2-thioxo-2,3-dihydroquinazolin-4(1H)-one (20.4 g, 82% yield) as an off-white solid. MS: (ES⁺) m/z=286.0 [M+H]⁺.

### Step 2: Preparation of 8-acetyl-3,6-dimethyl-2-thioxo-2,3-dihydroquinazolin-4(1H)-one

A solution of 8-bromo-3,6-dimethyl-2-thioxo-2,3-dihydroquinazolin-4(1H)-one (10 g, 35.1 mmol) in dioxane (100 mL) was treated with tributyl(1-ethoxyethenyl)stannane (25.4 g, 70.1 mmol) for 3 min at room temperature under an argon atmosphere followed by the addition of Pd(PPh₃)₄ (8.1 g, 7 mmol) in portions at room temperature. The resulting mixture was stirred overnight at 100° C. under an argon atmosphere. To the above mixture was added aqueous 1 N HCl (100 mL) dropwise over 1 min at room temperature. The resulting mixture was stirred for 1 h at 50° C. The resulting mixture was concentrated under reduced pressure. The crude product was used in the next step directly without further purification. This resulted in 8-acetyl-3,6-dimethyl-2-thioxo-2,3-dihydroquinazolin-4(1H)-one (2 g, 23% yield) as a light-yellow solid. MS: (ES⁺) m/z=249.2 [M+H]⁺.

### Step 3: Preparation of 8-acetyl-2-((4-chloro-3-(trifluoromethoxy)benzyl)thio)-3,6-dimethylquinazolin-4(3H)-one

A solution of 8-acetyl-3,6-dimethyl-2-thioxo-2,3-dihydroquinazolin-4(1H)-one (2 g, 8.1 mmol) in THE (100 mL) was treated with 4-(bromomethyl)-1-chloro-2-(trifluoromethoxy)benzene (2.45 g, 8.5 mmol) for 1 min at room temperature under an argon atmosphere followed by the addition of Cs₂CO₃ (5.27 g, 16.1 mmol) in portions at room temperature. The resulting mixture was stirred for 2 h at 80° C. under an argon atmosphere. The resulting mixture was concentrated under vacuum after filtration to remove the solids. The residue was purified by silica gel column chromatography (eluted with CH₂Cl₂) to afford 8-acetyl-2-((4-chloro-3-(trifluoromethoxy)benzyl)thio)-3,6-dimethylquinazolin-4(3H)-one (0.85 g, 23% yield) as a light yellow solid. MS: (ES⁺) m/z=457.1 [M+H]⁺.

### Step 4: Preparation of 2-((1-(2-((4-chloro-3-(trifluoromethoxy)benzyl)thio)-3,6-dimethyl-4-oxo-3,4-dihydroquinazolin-8-yl)ethyl)amino)benzoic acid, Enantiomers 1 and 2

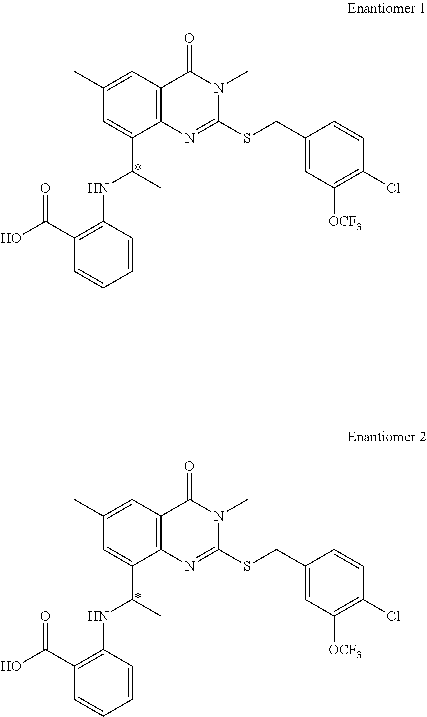 [SMILES: Cc1cc(C(C)Nc2ccccc2C(=O)O)c2nc(SCc3ccc(Cl)c(OC(F)(F)F)c3)n(C)c(=O)c2c1 ; Cc1cc(C(C)Nc2ccccc2C(=O)O)c2nc(SCc3ccc(Cl)c(OC(F)(F)F)c3)n(C)c(=O)c2c1]

The title compounds were prepared using methods similar to those described in Examples 7 and 8 to afford Example 31: 2-((1-(2-((4-chloro-3-(trifluoromethoxy)benzyl)thio)-3,6-dimethyl-4-oxo-3,4-dihydroquinazolin-8-yl)ethyl)amino)benzoic acid, Enantiomer 1 (62.7 mg, 42% yield, 99% ee) as a white solid. ¹H NMR (methanol-d₄, 400 MHz): δ 7.89-7.80 (m, 2H), 7.57 (m, 1H), 7.52 (m, 2H), 7.45 (m, 1H), 7.02 (m, 1H), 6.47 (m, 1H), 6.16-6.09 (m, 1H), 5.35 (m, 1H), 4.66 (m, 2H), 3.66 (s, 3H), 2.34 (s, 3H), 1.47 (m, 3H), MS: (ES⁺) m/z=578.1 [M+H]⁺; and Example 32: 2-((1-(2-((4-chloro-3-(trifluoromethoxy)benzyl)thio)-3,6-dimethyl-4-oxo-3,4-dihydroquinazolin-8-yl)ethyl)amino)benzoic acid, Enantiomer 2 (54.8 mg, 37% yield, 96% ee) as a white solid. ¹H NMR (methanol-d₄, 400 MHz): δ 7.89-7.80 (m, 2H), 7.57 (m, 1H), 7.52 (m, 2H), 7.45 (m, 1H), 7.02 (m, 1H), 6.47 (m, 1H), 6.13 (m, 1H), 5.35 (m, 1H), 4.70 (m, 1H), 4.62 (m, 1H), 3.66 (m, 3H), 2.35 (s, 3H), 1.47 (m, 3H), MS: (ES⁺) m/z=578.1 [M+H]⁺.

### Example 33: 5-fluoro-2-((1-(6-fluoro-3-methyl-4-oxo-2-(4-(2,2,2-trifluoroethyl)piperazin-1-yl)-3,4-dihydroquinazolin-8-yl)ethyl)amino)benzoic acid (Enantiomer 1); and

### Example 34: 5-fluoro-2-((1-(6-fluoro-3-methyl-4-oxo-2-(4-(2,2,2-trifluoroethyl)piperazin-1-yl)-3,4-dihydroquinazolin-8-yl)ethyl)amino)benzoic acid (Enantiomer 2)

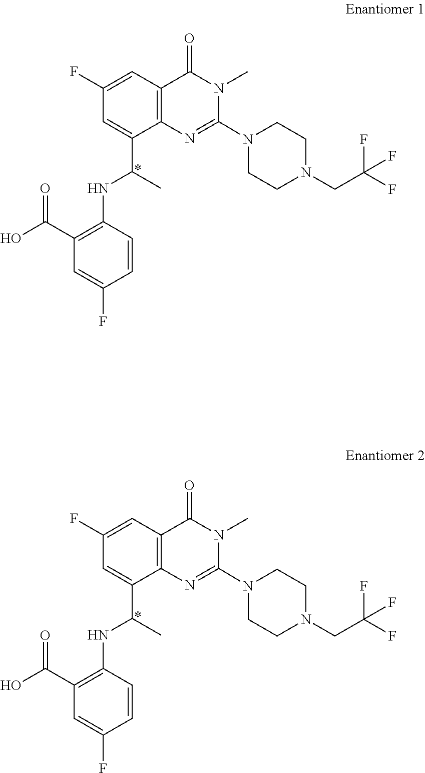 [SMILES: CC(Nc1ccc(F)cc1C(=O)O)c1cc(F)cc2c(=O)n(C)c(N3CCN(CC(F)(F)F)CC3)nc12 ; CC(Nc1ccc(F)cc1C(=O)O)c1cc(F)cc2c(=O)n(C)c(N3CCN(CC(F)(F)F)CC3)nc12]

The title compounds were prepared using methods similar to those described in Examples 7, 8, 11 and 12 to afford Example 33: 5-fluoro-2-((1-(6-fluoro-3-methyl-4-oxo-2-(4-(2,2,2-trifluoroethyl)piperazin-1-yl)-3,4-dihydroquinazolin-8-yl)ethyl)amino)benzoic acid, Enantiomer 1 (31.6 mg, 20% yield, 99% ee), ¹H NMR (400 MHz, methanol-d₄) δ 7.68-7.53 (m, 2H), 7.47-7.38 (m, 1H), 7.04-6.92 (m, 1H), 6.45-6.37 (m, 1H), 5.57-5.46 (m, 1H), 3.62 (s, 3H), 3.43-3.37 (m, 4H), 3.24-3.17 (m, 2H), 2.99-2.89 (m, 4H), 1.65 (s, 3H), MS: (ES⁺) m/z=526.4 [M+H]⁺; and Example 34: 5-fluoro-2-((1-(6-fluoro-3-methyl-4-oxo-2-(4-(2,2,2-trifluoroethyl)piperazin-1-yl)-3,4-dihydroquinazolin-8-yl)ethyl)amino)benzoic acid (Enantiomer 2) (26.0 mg, 16% yield, 99% ee), ¹H NMR (400 MHz, methanol-d₄) δ 7.68-7.57 (m, 2H), 7.47-7.37 (m, 1H), 7.02-6.92 (m, 1H), 6.47-6.38 (m, 1H), 5.55-5.46 (m, 1H), 3.63 (s, 3H), 3.44-3.37 (m, 4H), 3.24-3.17 (m, 2H), 2.99-2.88 (m, 4H), 1.65 (s, 3H), MS: (ES⁺) m/z=526.4 [M+H]⁺.

### Example 35: 2-((1-(6-fluoro-3-methyl-4-oxo-2-(4-(2,2,2-trifluoroethyl)piperazin-1-yl)-3,4-dihydroquinazolin-8-yl)ethyl)amino)benzoic acid (Enantiomer 1); and

### Example 36: 2-((1-(6-fluoro-3-methyl-4-oxo-2-(4-(2,2,2-trifluoroethyl)piperazin-1-yl)-3,4-dihydroquinazolin-8-yl)ethyl)amino)benzoic acid (Enantiomer 2)

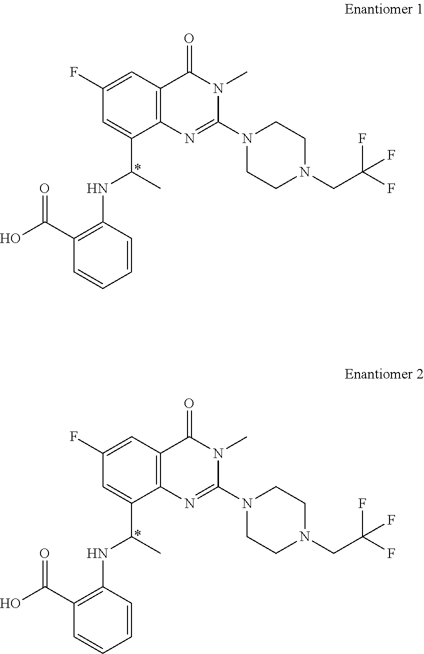 [SMILES: CC(Nc1ccccc1C(=O)O)c1cc(F)cc2c(=O)n(C)c(N3CCN(CC(F)(F)F)CC3)nc12 ; CC(Nc1ccccc1C(=O)O)c1cc(F)cc2c(=O)n(C)c(N3CCN(CC(F)(F)F)CC3)nc12]

### Step 1: Preparation of methyl 2-((1-(6-fluoro-3-methyl-4-oxo-2-(4-(2,2,2-trifluoroethyl)piperazin-1-yl)-3,4-dihydroquinazolin-8-yl)ethyl)amino)benzoate

Into a 250 mL round-bottom flask was added 6-fluoro-8-(1-hydroxyethyl)-3-methyl-2-(4-(2,2,2-trifluoroethyl)piperazin-1-yl)quinazolin-4(3H)-one (50 mg, 0.13 mmol) (prepared using methods similar to those described in Example 7) and CH₂Cl₂ (2 mL). To this mixture was added PBr₃ (87 mg, 0.32 mmol) dropwise at 0° C. under a nitrogen atmosphere. The resulting mixture was stirred overnight at room temperature under a nitrogen atmosphere. The resulting mixture was concentrated under reduced pressure. The crude product was dissolved in CH₂Cl₂ (50 mL). The mixture was basified to pH 7 via the addition of a saturated NaHCO₃ aqueous solution. The resulting mixture was extracted with DCM (2×50 mL) The combined organic layers were washed with brine (1×50 mL) and dried over anhydrous Na₂SO₄. After filtration, the filtrate was concentrated under reduced pressure to afford a crude product. The crude product was dissolved in ACN (10 mL). To the mixture was added methyl anthranilate (489 mg, 0.32 mmol) at room temperature. The resulting mixture was stirred for 2 h at 80° C. The resulting mixture was concentrated under reduced pressure. The residue was purified by silica gel column chromatography (eluted with PE:EA=2:1) to afford methyl 2-((1-(6-fluoro-3-methyl-4-oxo-2-(4-(2,2,2-trifluoroethyl)piperazin-1-yl)-3,4-dihydroquinazolin-8-yl)ethyl)amino)benzoate (75 mg, >95% crude yield) as a light yellow solid. MS: (ES⁺) m/z=522.3 [M+H]⁺.

### Step 2: Preparation of 2-((1-(6-fluoro-3-methyl-4-oxo-2-(4-(2,2,2-trifluoroethyl)piperazin-1-yl)-3,4-dihydroquinazolin-8-yl)ethyl)amino)benzoic acid, Enantiomers 1 and 2

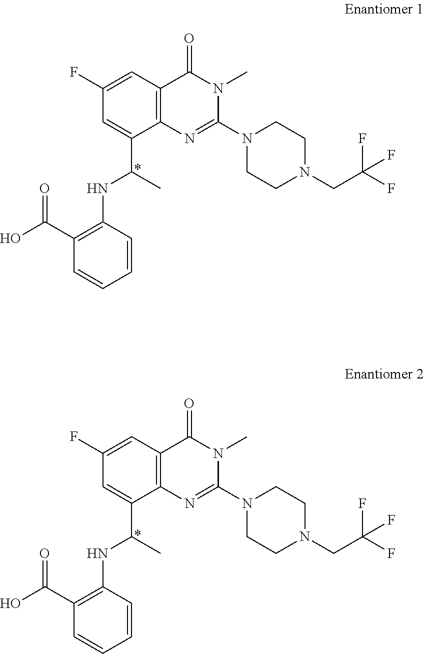 [SMILES: CC(Nc1ccccc1C(=O)O)c1cc(F)cc2c(=O)n(C)c(N3CCN(CC(F)(F)F)CC3)nc12 ; CC(Nc1ccccc1C(=O)O)c1cc(F)cc2c(=O)n(C)c(N3CCN(CC(F)(F)F)CC3)nc12]

The title compounds were prepared using methods similar to those described in Examples 7 and 8, using Intermediate 4, to afford Example 35: 2-((1-(6-fluoro-3-methyl-4-oxo-2-(4-(2,2,2-trifluoroethyl)piperazin-1-yl)-3,4-dihydroquinazolin-8-yl)ethyl)amino)benzoic acid, Enantiomer 1 (24 mg, 21% yield, 99% ee), 1H NMR (400 MHz, methanol-d₄) δ 7.96-7.85 (m, 1H), 7.68-7.57 (m, 1H), 7.49-7.37 (m, 1H), 7.21-7.08 (m, 1H), 6.61-6.47 (m, 1H), 6.47-6.35 (m, 1H), 5.60-5.44 (m, 1H), 3.62 (s, 3H), 3.46-3.35 (m, 4H), 3.25-3.11 (m, 2H), 3.02-2.81 (m, 4H), 1.64 (d, J=6.7 Hz, 3H), MS: (ES⁺) m/z=508.3 [M+H]⁺; and Example 36: 2-((1-(6-fluoro-3-methyl-4-oxo-2-(4-(2,2,2-trifluoroethyl)piperazin-1-yl)-3,4-dihydroquinazolin-8-yl)ethyl)amino)benzoic acid, Enantiomer 2 (11.5 mg, 10% yield, 99% ee), ¹H NMR (400 MHz, methanol-d₄) δ 7.95-7.86 (m, 1H), 7.66-7.57 (m, 1H), 7.47-7.37 (m, 1H), 7.19-7.07 (m, 1H), 6.60-6.48 (m, 1H), 6.47-6.35 (m, 1H), 5.58-5.44 (m, 1H), 3.62 (s, 3H), 3.47-3.35 (m, 4H), 3.25-3.11 (m, 2H), 3.05-2.77 (m, 4H), 1.64 (d, J=6.7 Hz, 3H), MS: (ES⁺) m/z=508.3 [M+H]⁺.

### Example 37: 2-((1-(3-methyl-4-oxo-2-(4-(2,2,2-trifluoroethyl)piperazin-1-yl)-6-(trifluoromethyl)-3,4-dihydroquinazolin-8-yl)ethyl)amino)benzoic acid (Enantiomer 1); and

### Example 38: 2-((1-(3-methyl-4-oxo-2-(4-(2,2,2-trifluoroethyl)piperazin-1-yl)-6-(trifluoromethyl)-3,4-dihydroquinazolin-8-yl)ethyl)amino)benzoic acid (Enantiomer 2)

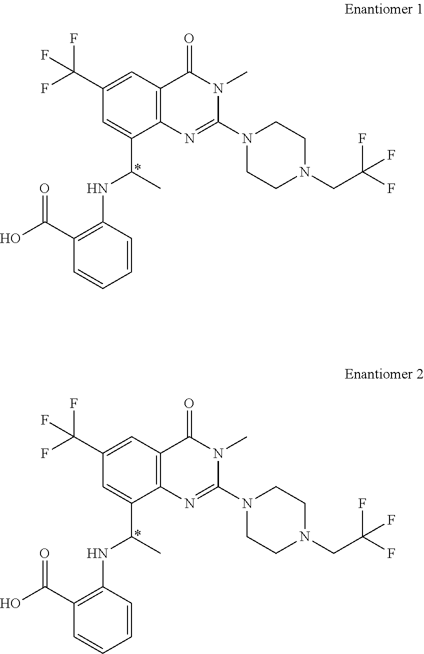 [SMILES: CC(Nc1ccccc1C(=O)O)c1cc(C(F)(F)F)cc2c(=O)n(C)c(N3CCN(CC(F)(F)F)CC3)nc12 ; CC(Nc1ccccc1C(=O)O)c1cc(C(F)(F)F)cc2c(=O)n(C)c(N3CCN(CC(F)(F)F)CC3)nc12]

The title compounds were prepared using methods similar to those described in Examples 35 and 36, using Intermediate 6, to afford Example 37: 2-((1-(3-methyl-4-oxo-2-(4-(2,2,2-trifluoroethyl)piperazin-1-yl)-6-(trifluoromethyl)-3,4-dihydroquinazolin-8-yl)ethyl)amino)benzoic acid, Enantiomer 1 (30 mg, 25% yield, 99% ee) as a white solid. ¹H NMR (400 MHz, DMSO-d₆) δ 12.68 (s, 1H), 8.45 (d, J=6.9 Hz, 1H), 8.14 (d, J=2.2 Hz, 1H), 7.84 (d, J=2.2 Hz, 1H), 7.81-7.75 (m, 1H), 7.24-7.14 (m, 1H), 6.52-6.46 (m, 2H), 5.48-5.35 (m, 1H), 3.48 (s, 3H), 3.44-3.36 (m, 4H), 3.29 (d, J=10.2 Hz, 2H), 2.90-2.75 (m, 4H), 1.61 (d, J=6.6 Hz, 3H), MS: (ES⁻) m/z=556.3 [M−H]; and Example 38: 2-((1-(3-methyl-4-oxo-2-(4-(2,2,2-trifluoroethyl)piperazin-1-yl)-6-(trifluoromethyl)-3,4-dihydroquinazolin-8-yl)ethyl)amino)benzoic acid, Enantiomer 2 (30 mg, 25% yield, 99% ee) as a white solid. ¹H NMR (400 MHz, DMSO-d₆) δ 12.73 (s, 1H), 8.51-8.45 (m, 1H), 8.14 (d, J=2.2 Hz, 1H), 7.84 (d, J=2.2 Hz, 1H), 7.81-7.74 (m, 1H), 7.26-7.13 (m, 1H), 6.52-6.45 (m, 2H), 5.46-5.38 (m, 1H), 3.48 (s, 3H), 3.45-3.37 (m, 4H), 3.29 (d, J=10.2 Hz, 2H), 2.90-2.75 (m, 4H), 1.61 (d, J=6.6 Hz, 3H), MS: (ES⁻) m/z=556.4 [M−H].

### Example 39: 2-((1-(2-(isobutylthio)-3,6-dimethyl-4-oxo-3,4-dihydroquinazolin-8-yl)ethyl)amino)benzoic acid (Enantiomer 1); and

### Example 40: 2-((1-(2-(isobutylthio)-3,6-dimethyl-4-oxo-3,4-dihydroquinazolin-8-yl)ethyl)amino)benzoic acid (Enantiomer 2)

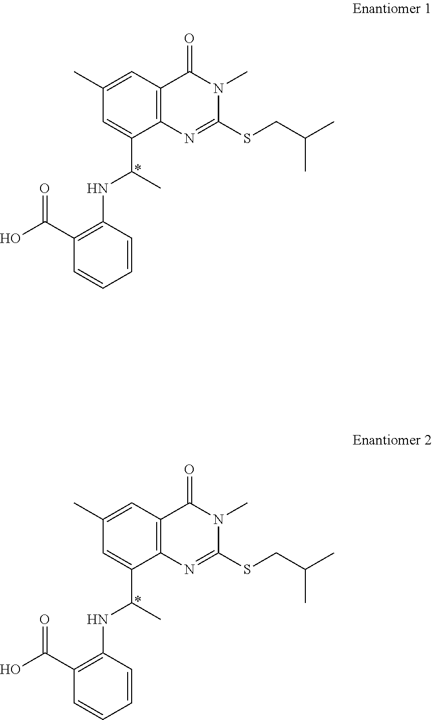 [SMILES: Cc1cc(C(C)Nc2ccccc2C(=O)O)c2nc(SCC(C)C)n(C)c(=O)c2c1 ; Cc1cc(C(C)Nc2ccccc2C(=O)O)c2nc(SCC(C)C)n(C)c(=O)c2c1]

The title compounds were prepared using methods similar to those described in Examples 31 and 32 to afford Example 39: 2-((1-(2-(isobutylthio)-3,6-dimethyl-4-oxo-3,4-dihydroquinazolin-8-yl)ethyl)amino)benzoic acid, Enantiomer 1 (6.3 mg, 5% yield, 99% ee) as a white solid. ¹H NMR (400 MHz, DMSO-d6) δ 8.60 (s, 1H), 7.84-7.74 (m, 2H), 7.51 (s, 1H), 7.15-7.11 (m, 1H), 6.51-6.47 (m, 1H), 6.31 (d, J=8.5 Hz, 1H), 5.50 (d, J=7.2 Hz, 1H), 3.55 (s, 3H), 3.24 (d, J=6.7 Hz, 2H), 2.33 (s, 3H), 2.13-2.06 (m, 1H), 1.54 (d, J=6.6 Hz, 3H), 1.07-1.01 (m, 6H), LCMS (ESI) m/z=426.0 (M+H), and Example 40: 2-((1-(2-(isobutylthio)-3,6-dimethyl-4-oxo-3,4-dihydroquinazolin-8-yl)ethyl)amino)benzoic acid, Enantiomer 2 (3.8 mg, 2% yield, 99% ee) as a white solid. ¹H NMR (400 MHz, DMSO-d₆) δ 8.66 (s, 1H), 7.82-7.79 (m, 1H), 7.77-7.75 (m, 1H), 7.51 (d, J=2.1 Hz, 1H), 7.14-7.09 (m, 1H), 6.53-6.44 (m, 1H), 6.30 (d, J=8.4 Hz, 1H), 5.50 (d, J=6.9 Hz, 1H), 3.55 (s, 3H), 3.24 (d, J=6.7 Hz, 2H), 2.33 (s, 3H), 2.15-2.04 (m, 1H), 1.54 (d, J=6.7 Hz, 3H), 1.07-1.01 (m, 6H), LCMS (ESI) m/z=426.0 (M+H).

### Example 41: 2-((1-(2-((4-chlorophenyl)amino)-3,6-dimethyl-4-oxo-3,4-dihydroquinazolin-8-yl)ethyl)amino)benzoic acid (Enantiomer 1); and

### Example 42: 2-((1-(2-((4-chlorophenyl)amino)-3,6-dimethyl-4-oxo-3,4-dihydroquinazolin-8-yl)ethyl)amino)benzoic acid (Enantiomer 2)

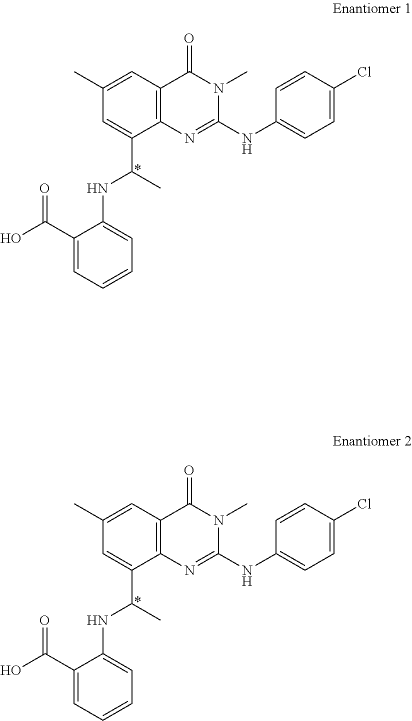 [SMILES: Cc1cc(C(C)Nc2ccccc2C(=O)O)c2nc(Nc3ccc(Cl)cc3)n(C)c(=O)c2c1 ; Cc1cc(C(C)Nc2ccccc2C(=O)O)c2nc(Nc3ccc(Cl)cc3)n(C)c(=O)c2c1]

### Step 1: Preparation of 8-bromo-3,6-dimethyl-2-thioxo-2,3-dihydroquinazolin-4(1H)-one

Into a 1000 mL round-bottom flask were added 2-amino-3-bromo-5-methylbenzoic acid (30.0 g, 130 mmol) in EtOH (500 mL), TEA (45 mL, 326 mmol) and methyl isothiocyanate (19.0 g, 261 mmol) at room temperature. The resulting mixture was stirred for 4 h at 100° C. under an argon atmosphere. The resulting mixture was concentrated under reduced pressure. The residue was dissolved in Et₂O (500 mL). The precipitated solids were collected by filtration and washed with Et₂O (2×300 mL). This resulted in 8-bromo-3,6-dimethyl-2-sulfanylidene-1H-quinazolin-4-one (31.8 g, 86% yield) as a brown solid. ¹H NMR (400 MHz, DMSO-d6) δ 10.74 (s, 1H), 7.76 (d, J=2.0 Hz, 1H), 7.73-7.70 (m, 1H), 3.66 (s, 3H), 2.33 (s, 3H).

### Step 2: Preparation of 8-bromo-3,6-dimethyl-2-(methylthio)quinazolin-4(3H)-one

To a stirred mixture of 8-bromo-3,6-dimethyl-2-sulfanylidene-1H-quinazolin-4-one (9.0 g, 31.5 mmol) and NaOH (2.52 g, 63.1 mmol) in DMF (90 mL) was added dimethyl sulfate (5.9 g, 47.3 mmol) in portions at room temperature under an argon atmosphere. The resulting mixture was stirred for 2 h at room temperature under an argon atmosphere. The mixture was cooled to 0° C. The reaction was quenched with ice water (50 mL) at 0° C. The resulting solid was washed five times with water (10 mL each), and then dried under reduced pressure. This resulted in 8-bromo-3,6-dimethyl-2-(methylthio)quinazolin-4(3H)-one (8.0 g, 85% yield) as a white solid. ¹H NMR (300 MHz, chloroform-d) δ 7.99-7.91 (m, 1H), 7.83-7.76 (m, 1H), 3.60 (s, 3H), 2.73 (s, 3H), 2.46-2.40 (m, 3H).

### Step 3: Preparation of 8-acetyl-3,6-dimethyl-2-(methylthio)quinazolin-4(3H)-one

To a stirred mixture of 8-bromo-3,6-dimethyl-2-(methylthio)quinazolin-4(3H)-one (7.1 g, 23.7 mmol) and tributyl(1-ethoxyethenyl)stannane (10.3 g, 28.5 mmol) in 1,4-dioxane (100 mL) was added tetrakis(triphenylphosphine)palladium (2.7 g, 2.4 mmol) in portions at room temperature under an argon atmosphere. The resulting mixture was stirred overnight at 100° C. The mixture was allowed to cool down to room temperature. To the mixture was added aqueous 1 N HCl (14.4 mL) in portions over 5 min at 0° C. The resulting mixture was stirred for 1 h at room temperature. The reaction was quenched with ice water (50 mL) at room temperature. The aqueous layer was extracted with EtOAc (3×50 mL). The resulting mixture was concentrated under reduced pressure. The residue was purified by silica gel column chromatography (eluted with PE:EA=1:0 and increased to PE:EA=5:1 over 30 min) to afford 8-acetyl-3,6-dimethyl-2-(methylthio)quinazolin-4(3H)-one (4.0 g, 64% yield) as a white solid. ¹H NMR (300 MHz, chloroform-d) δ 8.22-8.15 (m, 1H), 7.86-7.79 (m, 1H), 3.64 (s, 3H), 2.87 (s, 3H), 2.66 (s, 3H), 2.51-2.44 (m, 3H).

### Step 4: Preparation of 8-(1-hydroxyethyl)-3,6-dimethyl-2-(methylthio)quinazolin-4(3H)-one

A solution of 8-acetyl-3,6-dimethyl-2-(methylthio)quinazolin-4(3H)-one (2.9 g, 11.1 mmol) in MeOH (20 mL) was treated with NaBH₄ (836 mg, 22 mmol) for 5 min at 0° C. under a nitrogen atmosphere. The resulting mixture was stirred for 1 h at room temperature. The resulting mixture was concentrated under reduced pressure, then quenched with ice water (50 mL) at 0° C. The resulting mixture was extracted with CH₂Cl₂ (2×50 mL). The combined organic layers were washed with brine, dried over anhydrous Na₂SO₄, and concentrated under reduced pressure. The residue was purified by silica gel column chromatography (eluted with PE:EA=1:0 increased to PE:EA=5:1 over 30 min) to afford 8-(1-hydroxyethyl)-3,6-dimethyl-2-(methylthio)quinazolin-4(3H)-one (2.3 g, 79% yield) as a white solid. LCMS (ESI) m/z=265.2 (M+H).

### Step 5: Preparation of methyl 2-((1-(3,6-dimethyl-2-(methylthio)-4-oxo-3,4-dihydroquinazolin-8-yl)ethyl)amino)benzoate

A solution of 8-(1-hydroxyethyl)-3,6-dimethyl-2-(methylthio)quinazolin-4(3H)-one (2.5 g, 9.4 mmol) in DCM (30 mL) was treated with PBr₃ (25.6 g, 94.5 mmol) for 5 min at 0° C. under a nitrogen atmosphere. The resulting mixture was stirred for 3 h at room temperature. The reaction was quenched with ice water (100 mL). The resulting mixture was extracted with CH₂Cl₂ (2×100 mL). The combined organic layers were washed with brine, dried over anhydrous Na₂SO₄, and concentrated under reduced pressure. The residue was suspended in MeCN (30 mL) and K₂CO₃ (2.6 g, 18.9 mmol) and methyl anthranilate (4.3 g, 28.3 mmol) were added in portions at room temperature. The resulting mixture was stirred for 2 h at 80° C. The reaction was then cooled to room temperature and quenched with ice water (50 mL). The resulting mixture was extracted with CH₂Cl₂ (2×50 mL). The combined organic layers were washed with brine, dried over anhydrous Na₂SO₄, and concentrated under reduced pressure. The residue was purified by silica gel column chromatography (eluted with PE:EA=1:0 increased to PE:EA=5:1 over 30 min) to afford methyl 2-((1-(3,6-dimethyl-2-(methylthio)-4-oxo-3,4-dihydroquinazolin-8-yl)ethyl)amino)benzoate (2.5 g, 67% yield) as a white solid. LCMS (ESI) m/z=397.9 (M+H).

### Step 6: Preparation of methyl 2-((1-(3,6-dimethyl-2-(methylsulfonyl)-4-oxo-3,4-dihydroquinazolin-8-yl)ethyl)amino)benzoate

A solution of methyl 2-((1-(3,6-dimethyl-2-(methylthio)-4-oxo-3,4-dihydroquinazolin-8-yl)ethyl)amino)benzoate (1.5 g, 3.7 mmol) in DMF (15 mL) was treated with m-CPBA (1.9 g, 11.3 mmol) for 5 min at 0° C. under a nitrogen atmosphere. The resulting mixture was stirred overnight at room temperature. The reaction was quenched with saturated NaHCO₃ solution (50 mL) at 0° C. The resulting mixture was extracted with CH₂Cl₂ (2×50 mL). The combined organic layers were washed with brine, dried over anhydrous Na₂SO₄ and concentrated under reduced pressure. The residue was purified by silica gel column chromatography (eluted with PE:EA=1:0 increased to PE:EA=5:1 over 30 min) to afford methyl 2-((1-(3,6-dimethyl-2-(methylsulfonyl)-4-oxo-3,4-dihydroquinazolin-8-yl)ethyl)amino)benzoate (911 mg, 56% yield) as an off-white solid. LCMS (ESI) m/z=430.0 (M+H).

### Step 7: Preparation of methyl 2-((1-(2-((4-chlorophenyl)amino)-3,6-dimethyl-4-oxo-3,4-dihydroquinazolin-8-yl)ethyl)amino)benzoate

To a mixture of methyl 2-((1-(3,6-dimethyl-2-(methylsulfonyl)-4-oxo-3,4-dihydroquinazolin-8-yl)ethyl)amino)benzoate (120 mg, 0.3 mmol) and 4-chloroaniline (107 mg, 0.8 mmol) in THE (4.0 mL) was added LiHMDS in THE (2 M, 1.2 mL) dropwise at 0° C. under an argon atmosphere. The resulting mixture was then warmed to room temperature and stirred for 30 min. The reaction was quenched with ice water (20 mL). The resulting mixture was extracted with EtOAc (2×20 mL). The combined organic layers were washed with brine, dried over anhydrous Na₂SO₄, and concentrated under reduced pressure. The residue was purified by silica gel column chromatography (eluted with PE:EA=1:0 increased to PE:EA=1:1 over 30 min) to afford methyl 2-((1-(2-((4-chlorophenyl)amino)-3,6-dimethyl-4-oxo-3,4-dihydroquinazolin-8-yl)ethyl)amino)benzoate (125 mg, 94% yield) as a white solid. ¹H NMR (300 MHz, DMSO-d₆) δ 8.82 (s, 1H), 8.14 (d, J=6.3 Hz, 1H), 7.79 (d, J=9.0 Hz, 3H), 7.70 (d, J=1.8 Hz, 1H), 7.38 (d, J=8.9 Hz, 3H), 7.22-7.11 (m, 1H), 6.57-6.46 (m, 1H), 6.29 (d, J=8.6 Hz, 1H), 5.32-5.22 (m, 1H), 3.83 (s, 3H), 3.63 (s, 3H), 2.29 (s, 3H), 1.50 (d, J=6.6 Hz, 3H).

### Step 8: Preparation of 2-((1-(2-((4-chlorophenyl)amino)-3,6-dimethyl-4-oxo-3,4-dihydroquinazolin-8-yl)ethyl)amino)benzoic acid (Enantiomers 1 and 2)

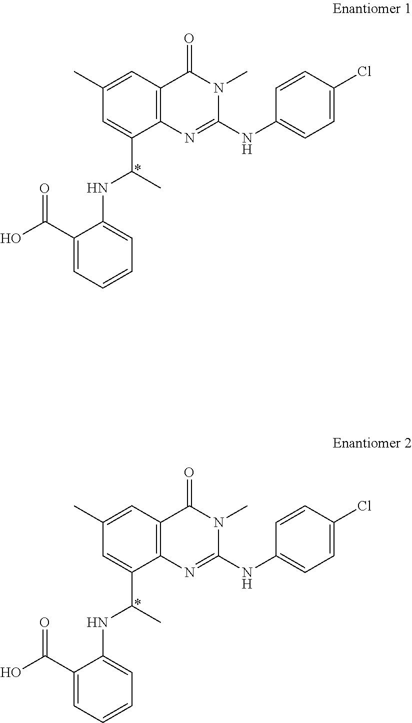 [SMILES: Cc1cc(C(C)Nc2ccccc2C(=O)O)c2nc(Nc3ccc(Cl)cc3)n(C)c(=O)c2c1 ; Cc1cc(C(C)Nc2ccccc2C(=O)O)c2nc(Nc3ccc(Cl)cc3)n(C)c(=O)c2c1]

The title compounds were prepared using methods similar to those described in Examples 7 and 8 to afford Example 41: 2-((1-(2-((4-chlorophenyl)amino)-3,6-dimethyl-4-oxo-3,4-dihydroquinazolin-8-yl)ethyl)amino)benzoic acid, Enantiomer 1 (29 mg, 36% yield, 99% ee) as a white solid. ¹H NMR (400 MHz, DMSO-d₆) δ 12.65 (s, 1H), 8.82 (s, 1H), 8.32 (s, 1H), 8.03-7.60 (m, 4H), 7.39 (d, J=6.7 Hz, 3H), 7.17-7.09 (m, 1H), 6.53-6.45 (m, 1H), 6.24 (d, J=8.6 Hz, 1H), 5.26 (s, 1H), 3.63 (s, 3H), 2.29 (s, 3H), 1.49 (d, J=6.6 Hz, 3H). LCMS (ESI) m/z=463.2 (M+H), and Example 42: 2-((1-(2-((4-chlorophenyl)amino)-3,6-dimethyl-4-oxo-3,4-dihydroquinazolin-8-yl)ethyl)amino)benzoic acid, Enantiomer 2 (29 mg, 36% yield, 99% ee) as a white solid. ¹H NMR (400 MHz, DMSO-d₆) δ 8.82 (s, 1H), 8.54 (s, 1H), 7.85-7.75 (m, 3H), 7.69 (d, J=2.0 Hz, 1H), 7.43-7.35 (m, 3H), 7.12-7.03 (m, 1H), 6.50-6.41 (m, 1H), 6.20 (d, J=8.5 Hz, 1H), 5.24 (s, 1H), 3.63 (s, 3H), 2.29 (s, 3H), 1.48 (d, J=6.6 Hz, 3H), LCMS (ESI) m/z=463.2 (M+H).

### Example 43: 2-((1-(2-((4-chlorobenzyl)amino)-3,6-dimethyl-4-oxo-3,4-dihydroquinazolin-8-yl)ethyl)amino)benzoic acid (Enantiomer 1); and

### Example 44: 2-((1-(2-((4-chlorobenzyl)amino)-3,6-dimethyl-4-oxo-3,4-dihydroquinazolin-8-yl)ethyl)amino)benzoic acid (Enantiomer 2)

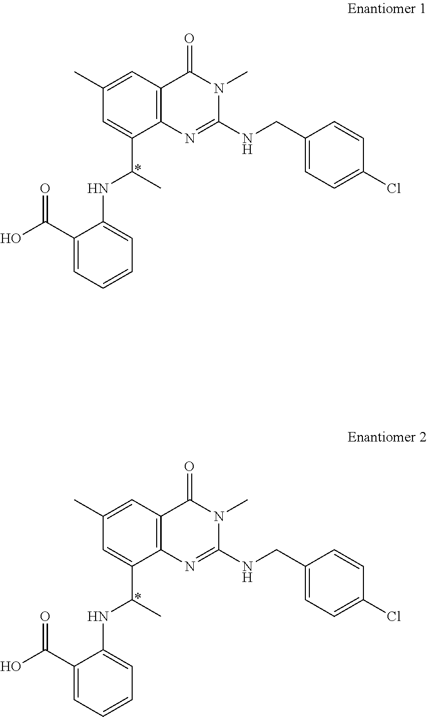 [SMILES: Cc1cc(C(C)Nc2ccccc2C(=O)O)c2nc(NCc3ccc(Cl)cc3)n(C)c(=O)c2c1 ; Cc1cc(C(C)Nc2ccccc2C(=O)O)c2nc(NCc3ccc(Cl)cc3)n(C)c(=O)c2c1]

The title compounds were prepared using methods similar to those described in Examples 41 and 42 to afford Example 43: 2-((1-(2-((4-chlorobenzyl)amino)-3,6-dimethyl-4-oxo-3,4-dihydroquinazolin-8-yl)ethyl)amino)benzoic acid, Enantiomer 1 (32 mg, 39% yield, 95% ee) as a white solid. ¹H NMR (400 MHz, DMSO-d₆) δ 8.44 (s, 1H), 7.85-7.78 (m, 1H), 7.75 (d, J=7.9 Hz, 1H), 7.58 (s, 1H), 7.44 (d, J=8.1 Hz, 2H), 7.32 (d, J=8.2 Hz, 2H), 7.26 (d, J=2.2 Hz, 1H), 7.06-6.97 (m, 1H), 6.48-6.40 (m, 1H), 6.11 (d, J=8.5 Hz, 1H), 5.19 (d, J=6.4 Hz, 1H), 4.74-4.64 (m, 1H), 4.60-4.51 (m, 1H), 3.50 (s, 3H), 2.22 (s, 3H), 1.25 (d, J=6.6 Hz, 3H). LCMS (ESI) m/z=476.9/479.0 (M+H), and Example 44: 2-((1-(2-((4-chlorobenzyl)amino)-3,6-dimethyl-4-oxo-3,4-dihydroquinazolin-8-yl)ethyl)amino)benzoic acid, Enantiomer 2 (30 mg, 36% yield, 95% ee) as a white solid. ¹H NMR (400 MHz, DMSO-d₆) δ 8.40 (s, 1H), 7.85-7.78 (m, 1H), 7.77-7.71 (m, 1H), 7.58 (d, J=2.2 Hz, 1H), 7.44 (d, J=8.2 Hz, 2H), 7.35-7.29 (m, 2H), 7.26 (d, J=2.2 Hz, 1H), 7.07-6.99 (m, 1H), 6.48-6.40 (m, 1H), 6.12 (d, J=8.5 Hz, 1H), 5.19 (d, J=6.4 Hz, 1H), 4.74-4.64 (m, 1H), 4.60-4.50 (m, 1H), 3.50 (s, 3H), 2.22 (s, 3H), 1.26 (d, J=6.6 Hz, 3H). LCMS (ESI) m/z=477.0/479.0 (M+H).

### Example 45: 2-((1-(3,6-dimethyl-4-oxo-2-(((R)-2-phenylpropyl)amino)-3,4-dihydroquinazolin-8-yl)ethyl)amino)benzoic acid (Enantiomer 1); and

### Example 46: 2-((1-(3,6-dimethyl-4-oxo-2-(((R)-2-phenylpropyl)amino)-3,4-dihydroquinazolin-8-yl)ethyl)amino)benzoic acid (Enantiomer 2)

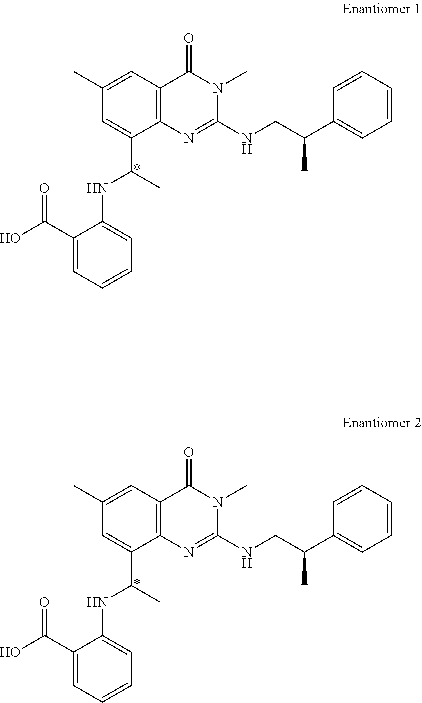 [SMILES: Cc1cc(C(C)Nc2ccccc2C(=O)O)c2nc(NC[C@H](C)c3ccccc3)n(C)c(=O)c2c1 ; Cc1cc(C(C)Nc2ccccc2C(=O)O)c2nc(NC[C@H](C)c3ccccc3)n(C)c(=O)c2c1]

The title compounds were prepared using methods similar to those described in Examples 41 and 42 to afford Example 45: 2-((1-(3,6-dimethyl-4-oxo-2-(((R)-2-phenylpropyl)amino)-3,4-dihydroquinazolin-8-yl)ethyl)amino)benzoic acid, Enantiomer 1 (25 mg, 36% yield, 99% ee) as a white solid. ¹H NMR (400 MHz, DMSO-d₆) δ 12.66 (s, 1H), 8.41 (s, 1H), 7.82-7.75 (m, 1H), 7.63-7.58 (m, 1H), 7.33 (d, J=2.2 Hz, 1H), 7.26 (d, J=4.3 Hz, 4H), 7.22-7.13 (m, 3H), 6.54-6.45 (m, 1H), 6.42 (d, J=8.5 Hz, 1H), 5.46 (m, 1H), 3.75-3.62 (m, 1H), 3.54-3.43 (m, 1H), 3.37 (m, 4H), 2.25 (s, 3H), 1.54 (d, J=6.6 Hz, 3H), 1.29 (d, J=6.9 Hz, 3H), LCMS (ESI) m/z=471.1 (M+H); and Example 46: 2-((1-(3,6-dimethyl-4-oxo-2-(((R)-2-phenylpropyl)amino)-3,4-dihydroquinazolin-8-yl)ethyl)amino)benzoic acid, Enantiomer 2 (16 mg, 23% yield, 99% ee) as a white solid. ¹H NMR (400 MHz, DMSO-d₆) δ 12.66 (s, 1H), 8.44 (s, 1H), 7.80-7.73 (m, 1H), 7.61 (d, J=1.8 Hz, 1H), 7.36-7.26 (m, 5H), 7.26-7.18 (m, 2H), 7.15-7.07 (m, 1H), 6.50-6.42 (m, 1H), 6.39 (d, J=8.6 Hz, 1H), 5.46 (m, 1H), 3.69-3.61 (m, 1H), 3.61-3.54 (m, 1H), 3.38 (m, 4H), 2.26 (s, 3H), 1.56 (d, J=6.6 Hz, 3H), 1.25 (d, J=10.5 Hz, 3H), LCMS (ESI) m/z=471.1 (M+H).

### Example 47: 2-((1-(3,6-dimethyl-4-oxo-2-(((S)-2-phenylpropyl)amino)-3,4-dihydroquinazolin-8-yl)ethyl)amino)benzoic acid (Enantiomer 1); and

### Example 48: 2-((1-(3,6-dimethyl-4-oxo-2-(((S)-2-phenylpropyl)amino)-3,4-dihydroquinazolin-8-yl)ethyl)amino)benzoic acid (Enantiomer 2)

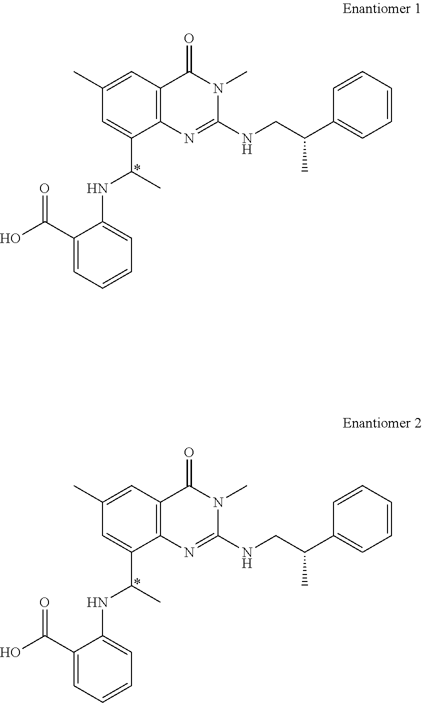 [SMILES: Cc1cc(C(C)Nc2ccccc2C(=O)O)c2nc(NC[C@@H](C)c3ccccc3)n(C)c(=O)c2c1 ; Cc1cc(C(C)Nc2ccccc2C(=O)O)c2nc(NC[C@@H](C)c3ccccc3)n(C)c(=O)c2c1]

The title compounds were prepared using methods similar to those described in Examples 41 and 42 to afford Example 47: 2-((1-(3,6-dimethyl-4-oxo-2-(((S)-2-phenylpropyl)amino)-3,4-dihydroquinazolin-8-yl)ethyl)amino)benzoic acid, Enantiomer 1 (30 mg, 56% yield, 99% ee) as a yellow solid. ¹H NMR (400 MHz, DMSO-d₆) δ 12.64 (s, 1H), 8.41 (s, 1H), 7.79 (d, J=8.0, 1.7 Hz, 1H), 7.61 (m, 1H), 7.33 (d, J=2.2 Hz, 1H), 7.26 (d, J=4.4 Hz, 4H), 7.22-7.13 (m, 3H), 6.55-6.45 (m, 1H), 6.42 (d, J=8.4 Hz, 1H), 5.46 (m, 1H), 3.74-3.64 (m, 1H), 3.54-3.43 (m, 1H), 3.37 (m, 4H), 2.25 (s, 3H), 1.54 (d, J=6.7 Hz, 3H), 1.29 (d, J=7.2 Hz, 3H), LCMS (ESI) m/z=471.2 (M+H); and Example 48: 2-((1-(3,6-dimethyl-4-oxo-2-(((S)-2-phenylpropyl)amino)-3,4-dihydroquinazolin-8-yl)ethyl)amino)benzoic acid Enantiomer 2 (12 mg, 24% yield, 99% ee) as a yellow solid. ¹H NMR (400 MHz, DMSO-d₆) δ 12.62 (s, 1H), 8.40 (s, 1H), 7.80-7.73 (m, 1H), 7.61 (d, J=2.1 Hz, 1H), 7.37-7.25 (m, 5H), 7.27-7.18 (m, 2H), 7.15-7.10 (m, 1H), 6.55-6.36 (m, 2H), 5.45 (m, 1H), 3.70-3.59 (m, 1H), 3.57-3.46 (m, 1H), 3.38 (m, 4H) 2.26 (s, 3H), 1.56 (d, J=6.7 Hz, 3H), 1.26 (d, J=7.2 Hz, 3H), LCMS (ESI) m/z=471.2 (M+H).

### Example 49: 2-((1-(3-cyclopropyl-2-(4,4-dimethylpiperidin-1-yl)-6-methyl-4-oxo-3,4-dihydroquinazolin-8-yl)ethyl)amino)benzoic acid (Enantiomer 1), and

### Example 50: 2-((1-(3-cyclopropyl-2-(4,4-dimethylpiperidin-1-yl)-6-methyl-4-oxo-3,4-dihydroquinazolin-8-yl)ethyl)amino)benzoic acid (Enantiomer 2)

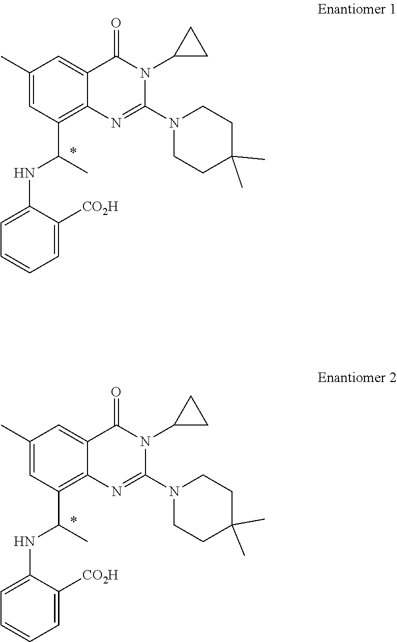

The title compounds were prepared using methods similar to those described in Examples 7 and 8, utilizing Intermediate 7, to afford Example 49: 2-((1-(3-cyclopropyl-2-(4,4-dimethylpiperidin-1-yl)-6-methyl-4-oxo-3,4-dihydroquinazolin-8-yl)ethyl)amino)benzoic acid, Enantiomer 1 (20 mg, 99% ee) as a white solid. ¹H NMR (400 MHz, DMSO-d₆) δ 12.61 (s, 1H), 8.38 (s, 1H), 7.80-7.73 (m, 1H), 7.64 (d, J=2.4 Hz, 1H), 7.39 (d, J=2.1 Hz, 1H), 7.22-7.13 (m, 1H), 6.53-6.44 (m, 2H), 5.35 (m, 1H), 3.52-3.36 (m, 4H), 3.22-3.13 (m, 1H), 2.28 (s, 3H), 1.58-1.41 (m, 7H), 1.20-1.10 (m, 2H), 1.00 (s, 6H), 0.81-0.73 (m, 1H), 0.73-0.62 (m, 1H), LCMS (ESI) m/z=475.3 (M+H); and Example 50: 2-((1-(3-cyclopropyl-2-(4,4-dimethylpiperidin-1-yl)-6-methyl-4-oxo-3,4-dihydroquinazolin-8-yl)ethyl)amino)benzoic acid, Enantiomer 2 (19 mg, 99% ee) as a white solid. ¹H NMR (400 MHz, DMSO-d₆) δ 12.61 (s, 1H), 8.38 (s, 1H), 7.80-7.73 (m, 1H), 7.66-7.60 (m, 1H), 7.39 (d, J=2.1 Hz, 1H), 7.22-7.13 (m, 1H), 6.53-6.44 (m, 2H), 5.35 (s, 1H), 3.48-3.41 (m, 4H), 3.22-3.13 (m, 1H), 2.28 (s, 3H), 1.56-1.42 (m, 7H), 1.19-1.13 (m, 2H), 1.00 (s, 6H), 0.75 (s, 1H), 0.67 (d, J=7.9 Hz, 1H), LCMS (ESI) m/z=475.2 (M+H).

### Example 51: 2-((1-(2-(4,4-dimethylpiperidin-1-yl)-3-(fluoromethyl)-6-methyl-4-oxo-3,4-dihydroquinazolin-8-yl)ethyl)amino)benzoic acid (Enantiomer 1); and

### Example 52: 2-((1-(2-(4,4-dimethylpiperidin-1-yl)-3-(fluoromethyl)-6-methyl-4-oxo-3,4-dihydroquinazolin-8-yl)ethyl)amino)benzoic acid (Enantiomer 2)

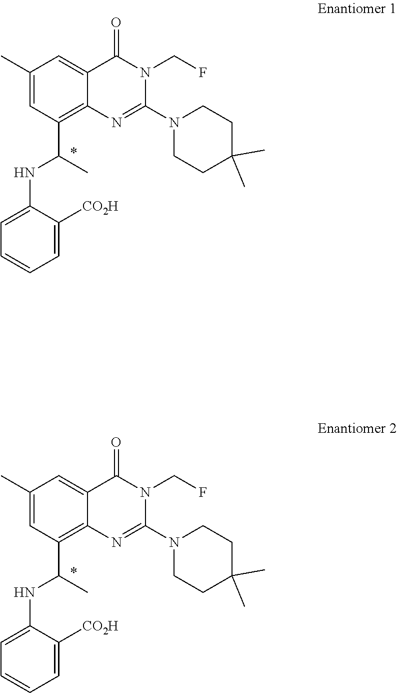

### Step 1: Preparation of 8-bromo-2-(4,4-dimethylpiperidin-1-yl)-6-methyl-3H-quinazolin-4-one

To a stirred solution of 8-bromo-2-chloro-6-methyl-3H-quinazolin-4-one (7.0 g, 25.6 mmol) and 4,4-dimethylpiperidine (2.9 g, 25.6 mmol) in NMP (100 mL) were added K₂CO₃ (14.2 g, 102 mmol) at room temperature. The resulting mixture was stirred overnight at 110° C. The reaction was cooled to room temperature and quenched with water (150 mL). The aqueous layer was extracted with EtOAc (3×150 mL). The combined organic layers were washed with brine and dried with anhydrous Na₂SO₄. The resulting mixture was concentrated under reduced pressure. The residue was purified by reversed-phase flash chromatography with the following conditions: column, C18 silica gel; mobile phase, MeCN in Water (0.1% FA), 10% to 95% gradient in 30 min; detector, UV 254 nm. This resulted in 8-bromo-2-(4,4-dimethylpiperidin-1-yl)-6-methyl-3H-quinazolin-4-one (6.1 g, 68% yield) as a yellow solid. LCMS (ESI) m/z=350.1/352.3 (M+H).

### Step 2: Preparation of 8-bromo-2-(4,4-dimethylpiperidin-1-yl)-3-(fluoromethyl)-6-methylquinazolin-4-one

A solution of 8-bromo-2-(4,4-dimethylpiperidin-1-yl)-6-methyl-3H-quinazolin-4-one (1.2 g, 3.4 mmol) in DMF (15 mL) was treated with NaH (411 mg, 17.1 mmol, 60% in oil) for 10 min at 0° C. under a nitrogen atmosphere followed by dropwise addition of bromofluoromethane (1.9 g, 17.1 mmol). The resulting mixture was stirred for 4 h at 60° C. The reaction was cooled to room temperature and quenched with water (50 mL). The aqueous layer was extracted with EtOAc (3×50 mL). The combined organic layers were washed with brine, dried with Na₂SO₄, and concentrated under reduced pressure. The residue was purified by reversed-phase flash chromatography with the following conditions: column, C18 silica gel; mobile phase, MeCN in Water, 40% to 100% gradient in 20 min. This resulted in 8-bromo-2-(4,4-dimethylpiperidin-1-yl)-3-(fluoromethyl)-6-methylquinazolin-4-one (1.0 g, 76% yield) as a yellow solid. LCMS (ESI) m/z=382.1/384.3 (M+H).

### Step 3: Preparation of 2-((1-(2-(4,4-dimethylpiperidin-1-yl)-3-(fluoromethyl)-6-methyl-4-oxo-3,4-dihydroquinazolin-8-yl)ethyl)amino)benzoic acid (Enantiomers 1 and 2)

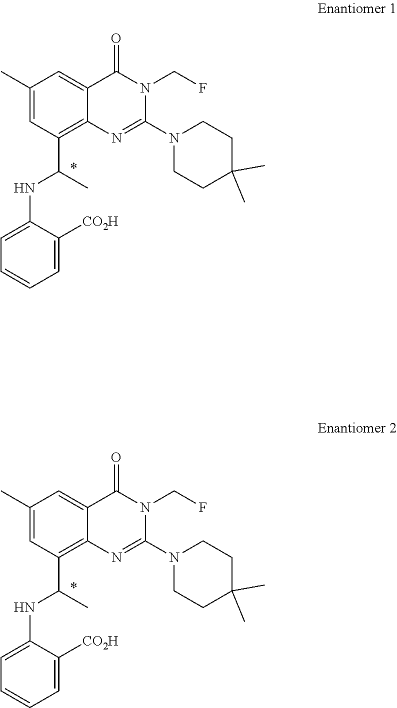

The title compounds were prepared using methods similar to those described in Examples 7 and 8 to afford Example 51: 2-((1-(2-(4,4-dimethylpiperidin-1-yl)-3-(fluoromethyl)-6-methyl-4-oxo-3,4-dihydroquinazolin-8-yl)ethyl)amino)benzoic acid, Enantiomer 1 (11 mg, 99% ee) as an white solid. ¹H NMR (400 MHz, DMSO-d₆) δ 12.63 (s, 1H), 8.46 (s, 1H), 7.80-7.75 (m, 1H), 7.58 (d, J=2.1 Hz, 1H), 7.48 (d, J=2.0 Hz, 1H), 7.18-7.12 (m, 1H), 6.51-6.40 (m, 2H), 6.33 (m, 1H), 6.20 (m, 1H), 5.43 (m, 1H), 3.9-3.83 (m, 4H), 2.31 (s, 3H), 1.57 (d, J=6.6 Hz, 3H), 1.45-1.36 (m, 4H), 1.00 (s, 6H), LCMS (ESI) m/z=467.2 (M+H); and Example 52: 2-((1-(2-(4,4-dimethylpiperidin-1-yl)-3-(fluoromethyl)-6-methyl-4-oxo-3,4-dihydroquinazolin-8-yl)ethyl)amino)benzoic acid, Enantiomer 2 (9 mg, 99% ee) as an white solid.

¹H NMR (400 MHz, DMSO-d₆) δ 12.63 (s, 1H), 8.46 (s, 1H), 7.80-7.75 (m, 1H), 7.58 (d, J=2.1 Hz, 1H), 7.48 (d, J=2.0 Hz, 1H), 7.18-7.12 (m, 1H), 6.51-6.40 (m, 2H), 6.33 (m, 1H), 6.20 (m, 1H), 5.43 (m, 1H), 3.9-3.83 (m, 4H), 2.31 (s, 3H), 1.57 (d, J=6.6 Hz, 3H), 1.45-1.36 (m, 4H), 1.00 (s, 6H); LCMS (ESI) m/z=467.2 (M+H).

### Assays and Compound Testing

### In Vitro Cell Proliferation: Determination of EC50 Values for Inhibition of Proliferation in T47D Cells Expressing Mutant PI3Ka (H1047R) Mutation and SKBR3 Cells Express WT PI3Ka.

T47D or SKBR3 cells were trypsinized, resuspended in culture media and seeded onto assay ready plates. T47D culture media consisted of RPMI, 10% FBS and Insulin (0.2 units/mL). SKBR3 culture media consisted of McCoys 5a and 10% FBS. Cells were seeded at a density of 1,500 cells/well and dispensed in 50 uL onto 384 well assay ready plates (Corning, 89089-790). Assay ready plates had previously been stamped with 10-point dilutions of compounds of interest, as well as controls. The Echo655 is used to stamp plates at 40 nL of compound or DMSO. Cells were grown for 72 hours at 37° Celsius and 5% CO₂. After 72 hours, cells were equilibrated at room temperature for 15 minutes. 30 uL of CellTiter-Glo reagent is added to the plate, which is then shaken for 30 minutes at temperature at 300-500 rpm. Cells are then read on an Envision plate reader. The percentage of inhibition of proliferation was calculated using the following formula: % Inhibition=100×(Lum_{D}−Lum_{Sample})/(Lum_{D}−Lum_{Inh}), where D is obtained from cells treated with 0.1% DMSO only; Inh is obtained from cells treated with 10 uM Alpelisib. The effective concentration achieving 50% inhibition of proliferation (EC50) is calculated by fitting the Curve using XWfit (v5.3.1.3), equation 201: Y=Bottom+(Top−Bottom)/(1+10\hat{ }((Log EC50−X)*HillSlope)).

### Reagent Table:

| Reagent | Vendor | Cat No. |
|---|---|---|
| Cell Titer Glo 2.0 Assay 500 mL | Promega | G9243 |
| Corning 384-well Low Flange Black Flat Bottom | Corning | 89089-790 |
| Polystyrene TC-treated Microplates, 10 per Bag, | | |
| with Lid, Sterile SKU: 3571 | | |
| PBS | Solarbio | P1020 |
| Trypan blue solution 0.4% | Fisher | T10282 |
| Fetal Bovine Serum Australia origin | Sigma | 82051-458 |
| Gibco RPMI 1640 Medium | Fisher | 11875093 |
| Gibco McCoy's 5A (modified) Medium, HEPES | Fisher | 12330031 |
| DMEM, high glucose, HEPES, no phenol red | Fisher | 21063045 |
| Insulin, human recombinant, zinc solution | Fisher | 12585-014 |

In Vitro Cell pAKT: Determination of IC50 Values for Inhibition of Phosphorylation of AKT (pAKT) in T47D Cells Expressing Mutant PI3Ka (H1047R) Mutation and SKBR3 Cells Express WT PI3Ka.

T47D or SKBR3 cells were trypsinized, resuspended in culture media and seeded onto assay ready plates. T47D culture media consisted of RPMI, 10% FBS and Insulin (0.2 units/mL). SKBR3 culture media consisted of McCoy's 5a and 10% FBS. Cells were seeded at a density of 5000 cells/well and dispensed in 12.5 uL onto 384 well assay ready plates (Perkin Elmer, 6008238)). Assay ready plates had previously been stamped with 10-point dilutions of compounds of interest, as well as controls. The Echo655 is used to stamp plates at 12.5 nL of compound or DMSO. Cells were grown for 6 hour at 37° Celsius and 5% CO₂. After 6 hour, 4 uL of Lysis buffer reagent was added to the plate, which was then centrifuged for 1 minute at 1000 rpm. Then the plate was incubated at room temperature for 30 minutes. After 30 minutes, 4 uL of antibody mix containing Eu Cryptate, d2 Cryptate, and detection buffer, was added to the plate. The plate was centrifuged for 1 minute at 1000 rpm and then incubated overnight at room temperature. The plate was read on an Envision plate reader using the HTRF protocol. The percentage of inhibition of AKT phosphorylation was calculated using the following formula: % Inhibition=100×(pAKTHC−pAKTSample)/(pAKTHC−pAKTLC)), where pAKTHC is obtained from cells treated with 0.1% DMSO only; pAKTLC is obtained from cells treated with 10 uM Alpelisib. The IC50 (concentration achieving 50% inhibition of pAKT) is calculated by fitting the Curve using XWfit (v5.3.1.3), equation 201: Y=Bottom+(Top−Bottom)/(1+10\hat{ }((Log IC50−X)*HillSlope)).

### Reagent Table:

| Reagent | Vendor | Cat No. |
|---|---|---|
| Gibco RPMI 1640 Medium, no phenol red | Fisher | 11835030 |
| Gibco RPMI 1640 Medium | Fisher | 11875093 |
| McCoy's 5A (Modified) Medium, HEPES | Fisher | 12330031 |
| DMEM, high glucose, HEPES, no phenol red | Fisher | 21063045 |
| Gibco Trypsin-EDTA (0.5%), no phenol red | Fisher | 15400054 |
| PBS | Solarbio | P1020 |
| Trypan blue solution 0.4% | Fisher | T10282 |
| Fetal Bovine Serum Australia origin | Sigma | 82051-458 |
| ProxiPlate-384 Plus, White, | Perkin | 6008238 |
| TC treated, Case of 160 | Elmer | |
| pAKT (Ser473) HTRF | Cisbio | 64AKSPEH |

For EC50 values shown in Table 1, “A” refers to 1 nM<EC50<500 nM; “B” refers to 500 nM<EC50<2 μM; “C” refers to 2 μM<EC50<15 μM; and “D” refers to an EC50>15 μM.

TABLE 1
Cellular proliferation data

| Example # | Avg T-47D EC50 | Avg SKBR3 EC50 |
|---|---|---|
| 1 | B | C |
| 2 | C | C |
| 3 | B | D |
| 4 | C | D |
| 5 | C | D |
| 6 | D | D |
| 7 | B | D |
| 8 | D | D |
| 9 | B | D |
| 10 | C | C |
| 11 | B | D |
| 12 | C | D |
| 13 | C | D |
| 14 | A | C |
| 15 | C | C |
| 16 | C | C |
| 17 | A | D |
| 18 | D | D |
| 19 | B | D |
| 20 | B | D |
| 21 | D | D |
| 22 | B | D |
| 23 | C | C |
| 24 | A | C |
| 25 | C | C |
| 26 | B | D |
| 27 | D | D |
| 28 | C | D |
| 29 | A | C |
| 30 | C | C |
| 31 | C | C |
| 32 | C | C |
| 33 | A | D |
| 34 | D | D |
| 35 | B | D |
| 36 | D | D |
| 37 | B | C |
| 38 | D | C |
| 39 | D | D |
| 40 | C | D |
| 41 | C | C |
| 42 | C | D |
| 43 | C | D |
| 44 | C | D |
| 45 | C | D |
| 46 | B | C |
| 47 | B | D |
| 48 | C | C |
| 49 | C | C |
| 50 | C | D |
| 51 | C | C |
| 52 | C | D |

For IC50 values shown in Table 2 “A” refers to 1 nM<IC50<1 μM; “B” refers to 1 μM<IC50<5 μM; “C” refers to 5 μM<IC50<15 μM; and “D” refers to an IC50>15 μM.

TABLE 2
Cellular pAKT data

| Example # | Avg T-47D IC50 | Avg SKBR3 IC50 |
|---|---|---|
| 1 | A | D |
| 2 | D | D |
| 3 | B | D |
| 4 | D | D |
| 5 | B | D |
| 6 | D | D |
| 7 | A | D |
| 8 | D | D |
| 9 | B | D |
| 10 | D | D |
| 11 | B | D |
| 12 | D | D |
| 13 | D | D |
| 14 | B | D |
| 17 | A | D |
| 18 | D | D |
| 20 | A | D |
| 21 | D | D |
| 22 | A | D |
| 23 | D | D |
| 24 | A | D |
| 25 | D | D |
| 26 | A | D |
| 27 | D | D |
| 28 | A | D |
| 29 | A | D |
| 30 | D | D |
| 31 | D | D |
| 32 | D | D |
| 33 | A | D |
| 34 | D | C |
| 35 | A | D |
| 36 | D | D |
| 37 | A | D |
| 38 | D | D |
| 39 | D | D |
| 40 | B | D |
| 41 | D | D |
| 42 | D | D |
| 43 | C | D |
| 44 | D | D |
| 45 | D | D |
| 46 | A | D |
| 47 | C | D |
| 48 | D | D |
| 49 | B | D |
| 50 | D | D |
| 51 | C | D |
| 52 | D | D |

CD1 mice were dosed with a single IV or PO dose, followed by serial sampling of plasma at 0.0833 (IV only), 0.25, 0.5, 1, 2, 4, 8, 24 hours post dose. Desired serial concentrations of working solutions were achieved by diluting stock solution of analyte with 50% acetonitrile in water solution. 10 μL of working solutions (0.5, 1, 2, 5, 10, 50, 100, 500, 1000 ng/mL) were added to 10 μL of the blank Female CD1 Mouse plasma to achieve calibration standards of 0.5~1000 ng/mL (0.5, 1, 2, 5, 10, 50, 100, 500, 1000 ng/mL) in a total volume of 20 IL. Five quality control samples at 1 ng/mL, 2 ng/mL, 5 ng/mL, 50 ng/mL and 800 ng/mL for plasma were prepared independently of those used for the calibration curves. These QC samples were prepared on the day of analysis in the same way as calibration standards. 20 μL standards, 20 μL QC samples and 20 μL unknown samples (10 μL plasma with 10 μL blank solution) were added to 200 μL of acetonitrile containing IS mixture for precipitating protein respectively. Then the samples were vortexed for 30 s. after centrifugation at 4° Celsius, 4000 rpm for 15 min. The supernatant was diluted with water at a ratio of 1:2 (V/V, 1:2), Then 5 μL of diluted supernatant was injected into the LC/MS/MS system for quantitative analysis. The results are shown in Table 3.

TABLE 3
mouse pharmacokinetic data

| Example # | 29 | 35 |
|---|---|---|
| PK-Mouse: | 1 | 1 |
| IV Dose | | |
| (mg/kg) | | |
| PK-Mouse: | 20% PEG400/20% PG/60% | 20% PEG400/20% PG/60% |
| IV Vehicle | of 20% HPBCD solution in | of 20% HPBCD solution in |
| | water | water |
| PK-Mouse: | solution | solution |
| IV Vehicle | | |
| appearance | | |
| PK-Mouse: | 8.7 | 59.5 |
| IV Cl | | |
| (mL/min/kg) | | |
| PK-Mouse: | 1741 | 242 |
| IV AUC last | | |
| (h*ng/mL) | | |
| PK-Mouse | 10 | 10 |
| PO Dose | | |
| (mg/kg) | | |
| PK-Mouse: | 30% SBEβCD in water | 30% SBEβCD in water |
| PO Vehicle | | |
| PK-Mouse: | suspension | suspension |
| PO Vehicle | | |
| appearance | | |
| PK-Mouse | 10697 | 4517 |
| PO AUC last | | |
| (h*ng/mL) | | |
| PK-Mouse: | 61 | 187 |
| PO F (%) | | |

### REFERENCES

Ali, K., Bilancio, A., Thomas, M., Pearce, W., Gilfillan, A. M., Tkaczyk, C., Kuehn, N., Gray, A., Giddings, J., Peskett, E., Fox, R., Bruce, I., Walker, C., Sawyer, C., Okkenhaug, K., Finan, P., and Vanhaesebroeck, B. 2004. Essential role for the p110delta phosphoinositide 3-kinase in the allergic response. Nature 431(7011): 1007-1011.
Backer, J. M. 2016. The intricate regulation and complex functions of the Class III phosphoinositide 3-kinase Vps34. Biochem J. 473(15): 2251-2271.
Castel, P., Toska, E., Engelman, J. A., and Scaltriti, M. 2021. The present and future of PI3K inhibitors for cancer therapy. Nat Cancer 2(6): 587-597.
Fruman, D. A., Chiu H., Hopkins B. D., Bagrodia, S., Cantley, L. C., and Abraham R. T. 2017. The PI3K pathway in human disease. Cell 170(4): 605-635.
Gadkar, K., Friedrich, C., Hurez, V., Ruiz, M. L., Dickmann, L., Jolly, M. K., Schutt, L., Jin, J., Ware, J. A., and Ramanujan, S. 2021. Quantitative systems pharmacology model-based investigation of adverse gastrointestinal events associated with prolonged treatment with PI3-kinase inhibitors. CPT Pharmacometrics Syst Pharmacol. 11(5): 616-627.
Goncalves, M. D., Hopkins, B. D., and Cantley, L. C. 2018. Phosphatidylinositol 3-Kinase, growth disorders, and cancer. N Engl J Med. 379(21): 2052-2062.
Hang, H. C. and Bertozzi, C. R. 2001. Chemoselective approaches to glycoproteon assembly. Accounts of Chemical Research 34(9): 727-736.
Hawkins, P. T. and Stephens, L. R. 2015. PI3K signalling in inflammation. 2015. Biochimica et Biophysica Acta 1851(6): 882-897.
Hopkins, B. D., Pauli, C., Du, X., Wang, D. G., Li, X., Wu, D., Amadiume, S. C., Goncalves, M. D., Hodakoski, C., Lundquist, M. R., Bareja, R., Ma, Y., Harris, E. M., Sboner, A., Beltran, H., Rubin, M. A., Mukherjee, S., and Cantley, L. C. 2018. Suppression of insulin feedback enhances the efficacy of PI3K inhibitors. Nature 560(7719): 499-503.
Jackson, S. P., Schoenwaelder, S. M., Goncalves, I., Nesbitt, W. S., Yap, C. L., Wright, C. E., Kenche, V., Anderson, K. E., Dopheide, S. M., Yuan, Y., Sturgeon, S. A., Prabaharan, H., Thompson, P. E., Smith, G. D., Shepherd, P. R., Daniele, N., Kulkarni, S., Abbott, B., Saylik, D., Jones, C., Lu, L., Giuliano, S., Hughan, S. C., Angus, J. A., Robertson, A. D., and Salem, H. H. 2005. PI 3-kinase p110beta: a new target for antithrombotic therapy. Nat Med. 11(5): 507-514.
Jiang, N., Dai, Q., Su, X., Fu, J., and Feng, X. 2020. Role of PI3K/AKT pathway in cancer: the framework of malignant behavior. Mol Biol Rep. 47(6): 4587-4629.
Keppler-Noreuil, K. M., Rios, J. J., Parker, V. E Semple, R. K., Lindhurst, M. J.; Sapp, J. C., Alomari, A., Ezaki, M, Dobyns, W., and Biesecker, L. G. 2015. PIK3CA-related overgrowth spectrum (PROS): diagnostic and testing eligibility criteria, differential diagnosis, and evaluation. Am J Mid Genet A. 167A(2): 287-295.
Kiick, K. L., Saxon, E., Tirrell, D. A., and Bertozzi, C. R. 2002. Incorporation of azides into recombinant proteins for chemoselective modification by the Staudinger ligation. Proc. Natl. Acad. Sci. U.S.A. 99(1): 19-24.
Klaus, S., Neumann, H., Zapf, A., Strübing, D., Hubner, S., Almena, J., Riermeier, T., Groß, P., Sarich, M., Krahnert, W., Rossen, K., and Beller, M. 2006. A general and efficient method for the formylation of aryl and heteroaryl bromides. Ang. Chem. Int. Ed. 45(1): 154-158.
Kurek, K. C., Luks, V. L, Ayturk, U. M., Alomari, A. I., Fishman, S. J., Spencer S. A., Mulliken, J. B., Bowen, M. E, Yamamoto, G. L., Kozakewich, H. P., and Warman, M. L 2012. Somatic mosaic activating mutations in PIK3CA cause CLOVES syndrome. Am J Hum Genet, 90(6): 1108-1115.
Lemieux, G. A. and Bertozzi, C. R. 1998. Chemoselective ligation reactions with proteins, oligosaccharides and cells. Trends in Biotechnology 16(12): 506-513.
Liu, P., Cheng, H., Roberts, T. M., and Zhao, J. J. 2009. Targeting the phosphoinositide 3-kinase pathway in cancer. Nat Rev Drug Discov. 8(8): 627-644.
Okkenhaug, K., Bilancio, A., Farjot, G., Priddle, H., Sancho, S., Peskett, E., Pearce, W., Meek, S. E., Salpekar, A., Waterfield, M. D., Smith, A. J., and Vanhaesebroeck, B. 2002. Impaired B and T cell antigen receptor signaling in p110delta PI 3-kinase mutant mice. Science 297(5583): 1031-1034.
Porta, C., Paglino, C., and Mosca, A. 2014. Targeting PI3K/Akt/mTOR signaling in cancer. Front Oncol. 4(64): 1-11.
Posor, Y., Eichhorn-Grunig, M., and Haucke, V. 2015. Phosphoinositides in endocytosis. Biochim Biophys Acta 2015 1851(6): 794-804.
Prakash, G., Krishnamurti, R., and Olah, G. 1989. Synthetic methods and reactions. 141. Fluoride-induced trifluoromethylation of carbonyl compounds with trifluormethyltrimethylsilane (TMS-CF₃). A trifluoromethide equivalent. J. Am. Chem. Soc. 111(1): 393-395.
Reichel, M. and Karaghiosoff, K. 2020. Reagents for selective fluoromethylation: A challenge in organofluorine chemistry. Ang. Chem. Int. Ed. 59(30): 12268-12281.
Rugo, H. S., Lacouture, M. E., Goncalves, M. D., Masharani, U., Aapro, M. S., and O'Shaughnessy, J. A. 2022. A multidisciplinary approach to optimizing care of patients treated with alpelisib. The Breast 61: 156-167.
Soler, A., Serra, H., Pearce, W., Angulo, A., Guillermet-Guibert, J., Friedman, L. S., Vinals, F., Gerhardt, H., Casanovas, O., Graupera, M., and Vanhaesebroeck, B. 2004. Inhibition of the p110α isoform of PI 3-kinase stimulates nonfunctional tumor angiogenesis. J Exp Med. 210(10): 1937-1945.
Thorpe, L. M., Yuzugullu, H., and Zhao, J. J. 2015. PI3K in cancer: divergent roles of isoforms, modes of activation and therapeutic targeting. Nat Rev Cancer 15(1): 7-24.
Vanhaesebroeck, B., Whitehead M. A., and Piñeiro, R. 2016. Molecules in medicine mini-review: isoforms of PI3K in biology and disease. J Mol Med (Berl). 94(1): 5-11.
Yang, J., Nie, J., Ma, X., Wei, Y., Peng, Y., and Wei, X. 2019. Targeting PI3K in cancer: mechanisms and advances in clinical trials. Mol Cancer 18(26): 1-28.
Zhao, Y., Huang, W., Zheng, J., and Hu, J. 2011. Efficient and direct nucleophilic difluoromethylation of carbonyl compounds and imines with Me₃SiCF₂H at ambient or low temperature. Org. Lett. 13(19): 5342-5345.

1.-37. (canceled)

38. A compound of Formula (1) 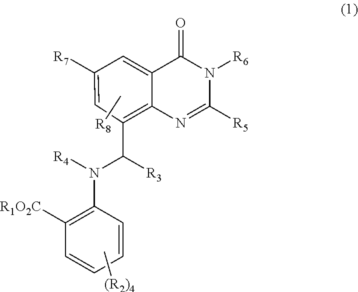
or a solvate, enantiomer, diastereomer, tautomer, polymorph or isotope-labeled compound,
or a pharmaceutically acceptable salt thereof,
wherein:
R₁ is H, C₁-C₄ alkyl or C₃-C₇ cycloalkyl;
each R₂ is independently H, C₁-C₄ alkyl, C₃-C₇ cycloalkyl, R₁₄—C≡C—, halogen, CN, CF₃, OCF₃, CFH₂ or CF₂H;
R₃ is H, C₁-C₄ alkyl, C₃-C₇ cycloalkyl, CF₃, CFH₂ or CF₂H, and where R₃ is not H, the carbon atom attached to R₃ is a chiral center and exists as a (R)- and (S)-racemic mixture or as either the (R)- or (S)-enantiomer;
R₄ is H or C₁-C₄ alkyl;
R₆ is H, C₁-C₄ alkyl, C₃-C₇ cycloalkyl, heteroaryl, CF₃, CFH₂ or CF₂H;
R₇ is H, C₁-C₄ alkyl, C₃-C₇ cycloalkyl, halogen, CN, CF₃, OCF₃, OCH₃, CFH₂ or CF₂H;
R₈ is H, C₁-C₄ alkyl, C₃-C₇ cycloalkyl, halogen, CN, CF₃, OCF₃, OCH₃, CFH₂ or CF₂H;
R₅ is 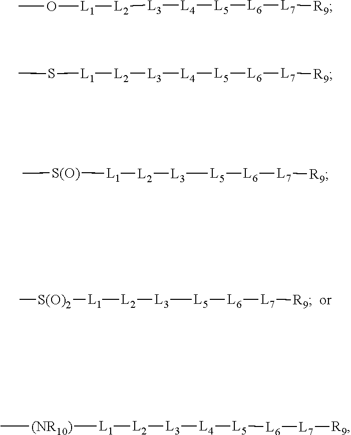
wherein:
each of L₁, L₂, L₃, L₆ and L₇ is independently (CHR₁₁), (CHR₁₁—O), (CHR₁₁—S), (C₃-C₇ cycloalkyl) or a bond;
L₄ is C═O, C═S or a bond;
L₅ is NR₁₀, S, O or a bond;
R₉ is H, C(═O)R₁₂, C(═O)NR₁₂R₁₃, C(═O)OR₁₂, C₁-C₆ alkyl, C₁-C₆ fluoroalkyl, cycloalkyl, heterocyclyl, aryl or heteroaryl, where each of the C₁-C₆ alkyl, C₁-C₆ fluoroalkyl, cycloalkyl, heterocyclyl, aryl or heteroaryl is unsubstituted or substituted, or alternatively when NR₁₀ is present, R₉ and R₁₀ together with the attached nitrogen atom may form a 4-7 membered non-aromatic heterocyclic ring, where the ring may contain additional heteroatoms and is substituted or unsubstituted;
each of R₁₀ and R₁₁ is H or C₁-C₄ alkyl, where the C₁-C₄ alkyl is unsubstituted or substituted; and
each of R₁₂ and R₁₃ is independently H or C₁-C₆ alkyl, cycloalkyl, heterocyclyl, aryl or heteroaryl, where each of the C₁-C₆ alkyl, cycloalkyl, heterocyclyl, aryl or heteroaryl is unsubstituted or substituted or alternatively, R₁₂ and R₁₃ together with the attached nitrogen atom together with the attached nitrogen atom may form a 4-7 membered non-aromatic heterocyclic ring, where the ring may contain additional heteroatoms and is substituted or unsubstituted;
each R₁₄ is independently H, C₁-C₃ alkyl or C₃-C₇ cycloalkyl;
or R₅ is
a non-aromatic N-linked heterocyclic ring 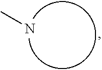
where the heterocyclic ring is substituted or unsubstituted, optionally contains one or more additional atoms selected from N (where the N is substituted or unsubstituted), O, Si (where the Si is substituted or unsubstituted) and S (where the S is oxidized or unoxidized), and is optionally part of a bridged, fused or spiro ring system.

39. The compound according to claim 38 or a solvate, enantiomer, diastereomer, tautomer, polymorph or isotope-labeled compound, or a pharmaceutically acceptable salt thereof, wherein R₅ is —(NR₁₀)-L₁-L₂-L₃-L₄-L₅-L₆-L₇-R₉, where L₁ to L₇, R₉ and R₁₀ are as defined.

40. The compound according to claim 38 or a solvate, enantiomer, diastereomer, tautomer, polymorph or isotope-labeled compound, or a pharmaceutically acceptable salt thereof, wherein R₅ is —O-L₁-L₂-L₃-L₄-L₅-L₆-L₇-R₉, where L₁ to L₇ and R₉ are as defined.

41. The compound according to claim 38 or a solvate, enantiomer, diastereomer, tautomer, polymorph or isotope-labeled compound, or a pharmaceutically acceptable salt thereof, wherein R₅ is —S-L₁-L₂-L₃-L₄-L₅-L₆-L₇-R₉; —S(O)-L₁-L₂-L₃-L₅-L₆-L₇-R₉; or —S(O)₂-L₁- L₂-L₃-L₅-L₆-L₇-R₉, where L₁ to L₇ and R₉ are as defined.

42. The compound according to claim 38 or a solvate, enantiomer, diastereomer, tautomer, polymorph or isotope-labeled compound, or a pharmaceutically acceptable salt thereof, wherein R₅ is a N-linked non-aromatic heterocyclic ring 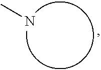
where the heterocyclic ring is substituted or unsubstituted, optionally contains one or more additional atoms selected from N (where the N is substituted or unsubstituted), O, Si (where the Si is substituted or unsubstituted) and S (where the S is oxidized or unoxidized), and is optionally part of a bridged, fused or spiro ring system.

43. The compound according to claim 42 or a solvate, enantiomer, diastereomer, tautomer, polymorph or isotope-labeled compound, or a pharmaceutically acceptable salt thereof, wherein the heterocyclic ring is substituted or unsubstituted, optionally contains one or more additional heteroatoms selected from N, O, Si and S, and is not part of a bridged, fused or spiro ring system.

44. The compound according to claim 42 or a solvate, enantiomer, diastereomer, tautomer, polymorph or isotope-labeled compound, or a pharmaceutically acceptable salt thereof, wherein the heterocyclic ring is substituted or unsubstituted, optionally contains one or more additional heteroatoms selected from N, O, Si and S, and is part of a bridged, fused or spiro ring system.

45. The compound according to claim 38 or a solvate, enantiomer, diastereomer, tautomer, polymorph or isotope-labeled compound, or a pharmaceutically acceptable salt thereof, wherein R₁ is H.

46. The compound according to claim 38 or a solvate, enantiomer, diastereomer, tautomer, polymorph or isotope-labeled compound, or a pharmaceutically acceptable salt thereof, wherein each R₂ is H.

47. The compound according to claim 38 or a solvate, enantiomer, diastereomer, tautomer, polymorph or isotope-labeled compound, or a pharmaceutically acceptable salt thereof, wherein R₃ is CH₃.

48. The compound according to claim 38 or a solvate, enantiomer, diastereomer, tautomer, polymorph or isotope-labeled compound, or a pharmaceutically acceptable salt thereof, wherein R₄ is H.

49. The compound according to claim 38 or a solvate, enantiomer, diastereomer, tautomer, polymorph or isotope-labeled compound, or a pharmaceutically acceptable salt thereof, wherein R₆ is CH₃.

50. The compound according to claim 38 or a solvate, enantiomer, diastereomer, tautomer, polymorph or isotope-labeled compound, or a pharmaceutically acceptable salt thereof, wherein R₇ is CH₃ or F.

51. The compound according to claim 38 or a solvate, enantiomer, diastereomer, tautomer, polymorph or isotope-labeled compound, or a pharmaceutically acceptable salt thereof, wherein R₈ is H.

52. The compound according to claim 38 or a solvate, enantiomer, diastereomer, tautomer, polymorph or isotope-labeled compound, or a pharmaceutically acceptable salt thereof, wherein R₁, each R₂ and R₄ are H.

53. The compound according to claim 38, wherein the compound of Formula (1) is the compound of Formula (2) 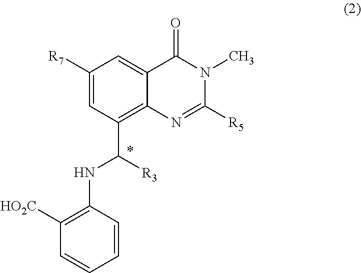
or a solvate, enantiomer, diastereomer, tautomer, polymorph or isotope-labeled compound, or a pharmaceutically acceptable salt thereof, wherein R₃ is CH₃, CF₃, CFH₂ or CF₂H; R₅ is defined as in the compound of Formula (1); R₇ is CH₃ or F; and the carbon marked with * is a chiral center and exists as a (R)- and (S)-racemic mixture or as either (R)- or (S)-enantiomer.

54. A pharmaceutical composition comprising the compound of claim 38 or a solvate, enantiomer, diastereomer, tautomer, polymorph or isotope-labeled compound, or a pharmaceutically acceptable salt thereof and a pharmaceutically acceptable carrier.

55. The pharmaceutical composition according to claim 54, further comprising one or more anti-cancer agents.

56. The pharmaceutical composition according to claim 55, wherein the one or more anti-cancer agents are selected from cyclophosphamide, dacarbazine, cisplatin, methotrexate, mercaptopurine, thioguanine, fluorouracil, cytarabine, vinblastine, paclitaxel, doxorubicin, bleomycin, mitomycin, prednisone, tamoxifen, flutamide, asparaginase, rituximab, trastuzumab, imatinib, retinoic acid, amifostine, camptothecin, topotecan, thalidomide, lenalidomide and a proteasome inhibitor.

57. A method of treating a disease in which PI3K activity is implicated in a subject in need of such treatment, the method comprising administering to the subject a therapeutically effective amount of the compound according to claim 38 or a solvate, enantiomer, diastereomer, tautomer, polymorph or isotope-labeled compound, or a pharmaceutically acceptable salt thereof.
